(19) United States
(12) Patent Application Publication
(10) Pub. No.: US 2023/0293427 A1
(43) Pub. Date: Sep. 21, 2023

(54) PREPARATIONS AND COMPOSITIONS COMPRISING POLYMER COMBINATION PREPARATIONS

(71) Applicant: SURGE Therapeutics, Inc., Cambridge, MA (US)

(72) Inventors: Michael Solomon Goldberg, Brookline, MA (US); Paul Adam Konowicz, Beverly, MA (US); Ivy Xiaoyu Chen, San Francisco, CA (US)

(21) Appl. No.: 18/016,096

(22) PCT Filed: Jul. 17, 2021
(86) PCT No.: PCT/US2021/042110

§ 371 Date: Jan. 13, 2023

Related U.S. Application Data
(60) Provisional application No. 63/108,861, filed on Nov. 2, 2020.
(60) Provisional application No. 63/053,488, filed on Jul. 17, 2020.

(51) Int. Cl. A61K 9/00; A61K 31/437; A61K 47/10; A61K 47/36

(52) U.S. Cl. CPC A61K 9/0024; A61K 31/437; A61K 47/10; A61K 47/36

(57) ABSTRACT

The present disclosure provides technologies related to certain polymer combination preparations and uses thereof. In many embodiments, such polymer combination preparations are temperature-responsive. In some embodiments, such polymer combination preparations may be useful as immunomodulatory biomaterials, e.g, to induce innate immunity or to resolve inflammation (e.g.; immunosuppressive inflammation). In some embodiments, such polymer combination preparations may be useful to formulate compositions comprising active agent(s).

### CROSS REFERENCE TO RELATED APPLICATIONS

This application claims the benefit of U.S. Provisional Application No. 63/053,488 filed Jul. 17, 2020, and U.S. Provisional Application No. 63/108,861 filed Nov. 2, 2020, the contents of each of which are hereby incorporated herein in their entirety.

### BACKGROUND

Surgery is often the first-line of treatment for solid tumor cancers and is generally used in combination with systemic administration of anti-cancer therapy. However, surgery-induced immunosuppression has been implicated in the development of post-operative septic complications and tumor metastasis due to changes in a variety of metabolic and endocrine responses, ultimately resulting in the death of many patients (Hiller, J.G. et al. Nature Reviews Clinical Oncology, 2018, 15, 205-218).

Systemic administration of medication, nutrition, or other substances into the circulatory system affects the entire body. Systemic routes of administration include enteral (e.g., oral dosage resulting in absorption of the drug through the gastrointestinal tract) and parenteral (e.g., intravenous, intramuscular, and subcutaneous injections) administration. Administration of immunotherapeutics typically relies on these systemic administration routes, which can lead to unwanted side effects. In some instances, certain promising therapeutics are extremely difficult to develop due to associated toxicities and the limitations of current administration methods and systems.

Hydrogels are a particularly attractive type of biomaterials, and have been used in a wide range of applications, including tissue engineering and regenerative medicine, diagnostics, cellular immobilization, and/or drug delivery. However, existing hydrogels also have several limitations that restricts the practical use of hydrogel-based drug delivery therapies. For example, many hydrogels are usually formed outside of the body and then implanted, since bulk hydrogels have a defined dimensionality, which may make extrusion through a needle challenging. While some hydrogels may be formed in situ, there may be potential risks and challenges associated with certain crosslinking agents, e.g., UV radiation and/or crosslinking chemicals.

### SUMMARY

The present inventor has previously described various systems involving an immunomodulatory biomaterial independent of an immunomodulatory payload (see, for example, PCT/US20/31169 filed May 1, 2020 published as WO2020/223698) or a combination of a biomaterial and an immunomodulatory payload (see, for example WO 2018/045058 or WO 2019/183216) that can be remarkably useful, among other things, when administered to subjects who have undergone or are undergoing tumor resection. Attributes of this system addressed the source of one or more problems associated with certain prior technologies including, for example, certain conventional approaches to cancer treatment. For example, this system could reduce and/or avoid certain adverse events (e.g., skin rashes, hepatitis, diarrhea, colitis, hypophysitis, thyroiditis, and adrenal insufficiency) that can be associated with systemic administration of immunotherapeutic agents. Among other things, this system could reduce or eliminate exposure of non-tumor-specific immune cells to systemically-administered immunotherapeutic drug(s) and/or to high doses of such drug(s) that are often required in order for systemic administration to achieve sufficient concentration in the tumor to induce a desired response; among other things, the system could provide local immunomodulation (e.g., local agonism of innate immunity) following tumor resection, which, among other things, can improve efficacy by concentrating the immunomodulatory effect where it is needed. Additionally or alternatively, such systems that provide local immunomodulation (e.g., agonism of innate immunity) following resection can, among other things, break local immune tolerance toward cancer and allow for development of systemic antitumor immunity, which can, for example, in some embodiments, lead to eradiation of disseminated disease.

The present disclosure provides an insight that certain such biomaterial formulations may be particularly useful and/or may provide particular beneficial effects, e.g., as described herein.

In some embodiments, the present disclosure identifies the source of a problem with certain prior technologies including, for example, with certain crosslinked biopolymer materials. Among other things, the present disclosure appreciates that certain crosslinking technologies may produce toxic by-products and/or may adversely affect stability and/or efficacy of agent(s) (e.g., therapeutic agents) that may be combined with biopolymer materials before or during crosslinking.

Alternatively or additionally, the present disclosure identifies the source of a problem with technologies that involve pre-forming (e.g., by cross-linking) a biopolymer material prior to introducing it into a subject. For example, the present disclosure appreciates that such pre-forming generates a material with a defined size and/or structure, which may restrict options for administration. The present disclosure provides technologies, including particular biomaterial preparations, that permit administration by a variety of routes and/or approaches, including by methods, such as injection and/or laparoscopic administration, that may be less invasive than implantation. In some such embodiments, preparations with improved administration characteristics may be administered in a liquid state; in some embodiments they may be administered in a pre-formed gel state characterized by flexible space-filling properties. In some such embodiments, provided preparations are comprised of a relevant material in particulate form (e.g., so that the preparations comprise a plurality of particles, e.g., characterized by a size distribution and/or other parameters as described herein).

Among other things, in some embodiments, the present disclosure provides temperature-responsive biomaterial preparations that, for example are able to transition from an injectable state to another state with material properties that provide beneficial effects, e.g., as described herein, without introduction of a cytotoxic crosslinking agent, e.g., UV radiation and/or small-molecule crosslinkers. Some such embodiments, thus provide valuable technologies for in situ formation of gelled materials, which technologies have various benefits relative to alternative technologies, and provide a solution to certain problems with such alternative technologies as identified herein. For example, the present disclosure identifies the source of a problem with various alternative technologies for in situ gelation, as many such technologies require treatments (e.g., exposure to UV radiation and/or to a small-molecule crosslinker, that may have toxic or otherwise damaging effects for the recipient and/or for an agent that may be included in or with the material.

In some embodiments, provided temperature-responsive biomaterial preparations (e.g., ones described herein) may demonstrate one or more immunomodulatory attributes, even in the absence of an immunomodulatory payload. For example, in some embodiments, provided temperature-responsive biomaterial preparations may promote innate immunity upon administration to a target site in subject in need thereof (e.g., tumor resection subjects).

In some embodiments, the present disclosure appreciates, among other things, that certain conventional preparations that are or comprise a poloxamer and that are used to form a hydrogel typically utilize such that are or comprise a poloxamer (e.g., Poloxamer 407 (P407)) at a minimum concentration of 16-20% (w/w). The present disclosure identifies the source of a problem with such conventional preparations, including that they may have certain disadvantages for administration to subjects, including, e.g., high solution viscosity that makes it less ideal for injection, and/or tissue irritation due to high concentrations of poloxamers. Moreover, the present disclosure demonstrates that it is possible to develop useful preparations with materially lower concentration(s) of such poloxamers.

For example, in some embodiments, the present disclosure provides an insight that certain poloxamers, e.g., Poloxamer 407 (P407), which have been typically used at a minimum concentration of 16-20% (w/w) to form a hydrogel, can form a useful temperature-responsive biomaterial at a concentration lower than 16% (w/w), including, e.g., lower than 14% (w/w), lower than 12% (w/w), lower than 11% (w/w), lower than 10.5% (w/w), lower than 10% (w/w), lower than 8% (w/w), lower than 6% (w/w), or lower, when combined with one or more biocompatible polymers. In some embodiments, such biocompatible polymers may be or comprise a polymer that is not temperature-responsive, e.g., in some embodiments which may be or comprise hyaluronic acid and/or chitosan or modified chitosan. In some embodiments, a biomaterial preparation comprising a poloxamer at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4%(w/w), or lower) and at least one additional polymer that is not poloxamer may be immunomodulatory itself in the absence of an immunomodulatory payload. For example, in some embodiments, such a biomaterial preparation may promote innate immunity upon administration to a target site in subject in need thereof (e.g., tumor resection subjects).

One aspect provided herein relates to a preparation or composition comprising a polymer combination preparation comprising at least first and second polymer components, the first polymer component is or comprises a poloxamer and the second polymer component is not a poloxamer, wherein the first polymer component is present in the polymer combination preparation at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4% (w/w), or lower). In some embodiments, a first polymer component is present in a polymer combination preparation at a concentration of 4% (w/w) to 11% (w/w), or 4% (w/w) to 10.5% (w/w), or 4% (w/w) to 10% (w/w). In some embodiments, a first polymer component is present in a polymer combination preparation at a concentration of 5% (w/w) to 11% (w/w), or 5% (w/w) to 10.5% (w/w), or 5% (w/w) to 10% (w/w). In some embodiments, a first polymer component is present in a polymer combination preparation at a concentration of 6% (w/w) to 11% (w/w), or 6% (w/w) to 10.5% (w/w), or 6% (w/w) to 10% (w/w). In some embodiments, such a polymer combination preparation is characterized in that it transitions from a precursor state to a polymer network state in response to a gelation trigger. Such a gelation trigger is or comprises one or more of the following: (a) temperature at or above critical gelation temperature (CGT) for the polymer combination preparation, (b) critical gelation weight ratio of the first polymer component to the second polymer component, (c) total polymer content, (d) molecular weights of the first and/or second polymer components, or (e) combinations thereof.

In some embodiments, crosslinks that form during the transition of the precursor state to the polymer network state do not comprise covalent crosslinks.

In many embodiments, such a polymer combination preparation is temperature-responsive. In some such embodiments, such a polymer combination preparation is characterized in that it transitions from a precursor state to a polymer network state in response to a temperature at or above CGT. For example, in some embodiments, the CGT for a provided polymer combination preparation is 18-39° C. In some embodiments the CGT for a provided polymer combination preparation is room temperature. In some embodiments, the CGT for a provided polymer combination preparation is 20-25° C. In some embodiments, the CGT for a provided polymer combination preparation is 25-30° C. In some embodiments the CGT for the polymer combination preparation is body temperature of a subject.

While many different poloxamers may be used in provided polymer combination preparations, in some embodiments, certain poloxamers, e.g., Poloxamer 407 (P407), Poloxamer 338 (P338), or Poloxamer 188 (P188) are particularly useful in certain polymer combination preparations described herein. For example, in some embodiments, poloxamer included as a first polymer component in a polymer combination preparation described herein is or comprises P407. In some embodiments, a first polymer component (e.g., comprising P407) is present in a provided polymer combination preparation at a concentration of 4% (w/w) to 12.5% (w/w), or 4% (w/w) to 11% (w/w), or 4% (w/w) to 10.5% (w/w), or 4% (w/w) to 10% (w/w). In some embodiments, a first polymer component (e.g., comprising P407) is present in a provided polymer combination preparation at a concentration of 5% (w/w) to 12.5% (w/w), or 5% (w/w) to 11% (w/w), or 5% (w/w) to 10.5% (w/w), or 5% (w/w) to 10% (w/w). In some embodiments, a first polymer component (e.g., comprising P407) is present in a provided polymer combination preparation at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a polymer combination preparation described herein comprises a total polymer content of at least 6% (w/w), at least 8% (w/w), at least 10% (w/w), at least 12%, or at least 15% (w/w). In some embodiments, a polymer combination preparation described herein comprises a total polymer content of 6% (w/w) to 20% (w/w), or 6% (w/w) to 15% (w/w), or 7% (w/w) to 15% (w/w). In some embodiments, a polymer combination preparation described herein comprises a total polymer content of 8% (w/w) to 20% (w/w), or 8% (w/w) to 15% (w/w), or 10% (w/w) to 15% (w/w).

In some embodiments, a polymer combination preparation described herein is characterized by a weight ratio of a first polymer component to a second polymer component of 1:1 to 14:1, or 1:1 to 10:1. In some embodiments, a polymer combination preparation described herein is characterized by a weight ratio of a first polymer component to a second polymer component of 1:1 to 1:3 or 1:1 to 1:2.

In some embodiments, a second polymer component in a provided polymer combination preparation is or comprises a carbohydrate polymer. Examples of a carbohydrate polymer that may be useful in accordance with the present disclosure include, but are not limited to, hyaluronic acid, chitosan, alginate, and variants and combinations thereof. In some embodiments, a carbohydrate polymer in a provided polymer combination preparation may be present at a concentration of below about 5% (w/w). In some embodiments, a carbohydrate polymer in a provided polymer combination preparation may be present at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

In some embodiments, a carbohydrate polymer that is useful for certain polymer combination preparations described herein is or comprises hyaluronic acid. In some embodiments, such hyaluronic acid may have an average molecular weight of 50 kDa to 2 MDa. In some embodiments, such hyaluronic acid may have an average molecular weight of 100 kDa to 500 kDa. In some embodiments, such hyaluronic acid may have an average molecular weight of 125 kDa to 375 kDa. In some embodiments, such hyaluronic acid may have an average molecular weight of 100 kDa to 400 kDa. In some embodiments, such hyaluronic acid may have an average molecular weight of 500 kDa to 1.5 MDa. In some embodiments, molecular weight of hyaluronic acid is characterized by weight average molecular weight. In some embodiments, molecular weight of hyaluronic acid is characterized by viscosity average molecular weight, which in some embodiments can be determined by converting intrinsic viscosity of hyaluronic acid to average molecular weight, for example, using the Mark-Houwink Equation. In some embodiments, molecular weight of hyaluronic acid can be measured by Size Exclusion Chromatography-Multiple Angle Laser Light Scattering (SEC-MALLS).

In some embodiments, number average molecular weight (Mn), weight average molecular weight (Mw), and/or dispersity (as characterized by polydispersity index) can be determined using SEC-MALLS.

In some embodiments, a carbohydrate polymer that is useful for certain polymer combination preparations described herein is or comprises a chitosan or a modified chitosan. In some embodiments, an exemplary modified chitosan is or comprises carboxymethyl chitosan.

In some embodiments, a preparation or composition comprising a polymer combination preparation as utilized and/or described herein in a precursor state. In some embodiments, a preparation or composition comprising a polymer combination preparation as utilized and/or described herein in a polymer network state (e.g., having one or more characteristics as described herein).

In some embodiments, a polymer network state is or comprises a viscous solution or colloid. In some embodiments, such a polymer network state may be characterized by a storage modulus of 100 Pa to 500 Pa. In some embodiments, a polymer network state is or comprises a hydrogel. In some embodiments, such a polymer network state may be characterized by a storage modulus of 500 Pa to 10,000 Pa, or 750 Pa to 7500 Pa.

In some embodiments, a polymer network state of a provided polymer combination preparation is characterized by a storage modulus that is at least 40% lower than that of a hydrogel formed from a P407 solution at a concentration of 18% (w/w). In some embodiments, a polymer network state of a provided polymer combination preparation, which its precursor state has been stored at a temperature that is below CGT (e.g., 2-8° C.) over a period of 1 month or longer, is characterized by a storage modulus, for example, as measured at 37° C., that maintains substantially the same (e.g., within 20%, within 10%, within 5%, or lower), as compared to that of a polymer network formed from a precursor state of such a provided polymer combination preparation that is freshly prepared. As will be understood by those skilled in the art, storage modulus of a biomaterial may be affected by biodegradation, chemical degradation (e.g., oxidation), and/or phase separation of polymer components in a combination.

In some embodiments, a polymer combination preparation as described and/or utilized herein has pH 5.0-8.5. In some embodiments, a polymer combination preparation as described and/or utilized herein has pH 7-8 (e.g., pH 7.4). For example, in some embodiments, a precursor state of a polymer combination preparation is a solution of the polymer combination preparation in a solvent system having pH 5.0-8.5 (e.g., in some embodiments pH 7-8). In some embodiments, such a solvent system is a buffered system. In some embodiments, such a buffered system may comprise one or more salts (e.g., but not limited to sodium phosphate, and/or sodium hydrogen carbonate). In some embodiments, such a solvent system is a buffer system having a higher buffering capacity than a 10 mM phosphate buffer. In some embodiments, such a solvent system is a buffer system having a higher buffering capacity than a 20 mM phosphate buffer.

In some embodiments, preparations or compositions described herein may be useful to provide sustained release of a payload incorporated therein. For example, in some embodiments, a provided polymer combination preparation in a polymer network state is characterized in that, when tested in vitro at 37° C., such polymer combination preparation releases a payload (e.g., a lipophilic agent) incorporated therein at a comparable rate as with a hydrogel formed from a P407 solution at a concentration of 18% (w/w). In some embodiments, a provided polymer combination preparation in a polymer network state is characterized in that, when tested in vitro at 37° C., no more than 40% of a payload (e.g., a lipophilic agent) incorporated in the polymer combination preparation is released within 24 hours. In some embodiments, a provided polymer combination preparation in a polymer network state is characterized in that, when tested in vitro at 37° C., more than 60% of a payload (e.g., a lipophilic agent) incorporated in the polymer combination preparation can be retained therein for at least 24 hours.

In some embodiments, a provided polymer combination preparation in a polymer network state is characterized in that, when tested in vitro at 37° C., such a polymer combination preparation releases a payload (e.g., a hydrophilic agent) incorporated therein at a comparable rate as, or at a faster rate than that of a hydrogel formed from a P407 solution at a concentration of 18% (w/w). In some embodiments, a provided polymer combination preparation in a polymer network state is characterized in that, when tested in vitro at 37° C., at least 40% of a payload (e.g., a hydrophilic agent) incorporated in the polymer combination preparation is released therefrom within 12 hours. In some embodiments, a provided polymer combination preparation in a polymer network state is characterized in that, when tested in vitro at 37° C., the polymer combination preparation releases a payload (e.g., a hydrophilic agent) incorporated therein at a faster rate (e.g., by at least 20% within 48 hours) as compared with that of a reference chemically crosslinked hydrogel. In some embodiments, such a reference chemically crosslinked hydrogel is or comprises a chemically crosslinked hyaluronic acid hydrogel, which is a hydrogel formed by mixing thiol-modified hyaluronic acid (Glycosil®) with a crosslinking agent, thiol-reactive PEGDA crosslinker (Extralink®), under conditions for gelation to occur.

In some embodiments, a preparation or composition described herein provides an immunomodulatory polymer combination preparation comprising a poloxamer (e.g., ones described herein) and a carbohydrate polymer (e.g., described herein), which is substantially free of an immunomodulatory payload. In some embodiments, such an immunomodulatory polymer combination preparation is characterized in that a test animal group with spontaneous metastases having, at a tumor resection site, such a polymer combination preparation in a polymer network state has a higher percent survival than a comparable test animal group having, at a tumor resection site, a poloxamer biomaterial, as assessed at 2 months after the administration.

In some embodiments, a preparation or composition described herein may comprise a polymer combination preparation (e.g., ones described herein) and one or more therapeutic agents, e.g., for treatment of a disease, disorder, or a condition (e.g., cancer). In some embodiments, one or more therapeutic agents that may be included in preparations or compositions described herein are or comprise one or more chemotherapeutic agents. In some embodiments, one or more therapeutic agents that may be included in preparations or compositions described herein are or comprise or more immunomodulatory payloads. Examples of immunomodulatory payloads that may be useful in accordance with the present disclosure include, but are not limited to activators of innate immune response, activators or adaptive immune response, modulators of macrophage effector function, modulators of inflammation, and combinations thereof.

In some embodiments, at least one therapeutic agent (e.g., at least one immunomodulatory payload) is incorporated in a polymer combination preparation described herein. In some embodiments, such a polymer combination preparation is characterized in that a test animal group with spontaneous metastases having, at a tumor resection site, the polymer combination preparation in the polymer network state has a higher percent survival than a comparable test animal group having, at a tumor resection site, a polymer combination preparation without the immunomodulatory payload, as assessed at 2 months or 3 months after the administration.

Preparations and/or compositions described herein can be useful for various medical applications, including, e.g., but not limited to immunomodulation and/or drug delivery. Thus, in some embodiments, preparations and/or compositions described herein can be formulated into pharmaceutical compositions for administration to subjects in need thereof. Accordingly, in one aspect, provided herein is a method comprising administering to a subject in need thereof a preparation or composition as described and/or utilized herein or a pharmaceutical compositions comprising the same.

In some embodiments, a preparation or composition as described and/or utilized herein or a pharmaceutical compositions comprising the same may be useful for treatment of cancer. In some such embodiments, a subject to be administered is a subject suffering from cancer. In some embodiments, a subject to be administered is a subject suffering from or susceptible to recurrent or disseminated cancer. In some embodiments, a subject to administered is a tumor resection subject.

In some embodiments, a method comprises administering a provided preparation or composition or a pharmaceutical composition comprising the same at a target site in a tumor resection subject. In some embodiments, such a preparation or composition or a pharmaceutical composition comprising the same is administered at a tumor resection site.

In some embodiments, administration may be performed by implantation. For example, in some embodiments, a preparation or composition comprising a polymer combination preparation in a polymer network state (e.g., a hydrogel) may be administered by implantation.

In some embodiments, administration may be performed by injection. In some embodiments, injection may be performed by a robotic arm. For example, in some embodiments, a preparation comprising a polymer combination preparation in a precursor state (e.g., a liquid state or an injectable state) is administered by injection, wherein the precursor state transitions to a polymer network state (e.g., a more viscous solution or colloid state or a hydrogel) upon the administration.

In some embodiments, administration may be performed concurrently with or subsequent to laparoscopy. In some embodiments, administration may be performed concurrently with or subsequent to a minimally invasive surgery (MIS), e.g., robot-assisted MIS, robotic surgery, and/or laparoscopic surgery, for tumor resection.

These, and other aspects encompassed by the present disclosure, are described in more detail below and in the claims.

### BRIEF DESCRIPTION OF THE DRAWINGS

FIGS. 1A-1B are heat maps depicting gelation properties of exemplary temperature-responsive polymer combination preparations comprising P407 at indicated concentrations in % (w/w) and hyaluronic acid (HA) having an average molecular weight of 1.5 MDa at indicated concentrations in % (w/w) in two different buffer systems. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted. FIG. 1A corresponds to 10 mM phosphate buffer saline (PBS) at pH 7.4. FIG. 1B corresponds to 0.1 M bicarbonate buffer at pH 8.0.

FIGS. 2A-2B are heat maps depicting gelation properties of exemplary temperature-responsive polymer combination preparations comprising P407 at indicated concentrations in % (w/w) and hyaluronic acid (HA) having an average molecular weight of 730 kDa at indicated concentrations in % (w/w) in two different buffer systems. Polymer combination preparations were exposed to a temperature of 37° C. to observe any gel formation. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted. FIG. 2A corresponds to 10 mM PBS at pH 7.4. FIG. 2B corresponds to 0.1 M bicarbonate buffer at pH 8.0.

FIG. 3 is a heat map depicting gelation properties of exemplary temperature-responsive polymer combination preparations comprising P407 at indicated concentrations in % (w/w) and modified chitosan (e.g., carboxymethyl chitosan; CMCH) at indicated concentrations in % (w/w) in 10 mM PBS at pH 7.4. Temperature-responsive polymer combination preparations were exposed to a temperature of 37° C. to observe any gel formation. A polymer combination preparation is determined to form a gel when such a polymer combination preparation becomes translucent or opaque, which is not flowable when angled or inverted.

FIGS. 4A-4B are graphical representations showing storage modulus of exemplary temperature-responsive polymer combination preparations after exposure to a temperature of 37° C., as compared to control polymer compositions. FIG. 4A: Linear scale. FIG. 4B: Logarithmic scale. Abbreviations: “18%P” = 18% (w/w) P407; “13.5%P + HA (10 mM PBS)” = 13.5% (w/w) P407 + 0.65% (w/w) 1.5 MDa HA in 10 mM PBS at pH 7.4; “13.5%P + HA (0.1 M bicar)” = 13.5% (w/w) P407 + 0.65% (w/w) 1.5 MDa HA in 0.1 M bicarbonate buffer at pH 8; “10%P + 1% HA (10 mM PBS)” = 10% (w/w) P407 + 1% (w/w) 1.5 MDa HA in 10 mM PBS at pH 7.4; “13.5%P + CMCH” = 13.5% (w/w) P407 + 1.3% (w/w) CMCH in 10 mM PBS at pH 7.4; “12.5% Extralink” = Chemically-crosslinked hyaluronic acid with 12.5% Extralink thiol crosslinker; “1.5% Extralink” = Chemically-crosslinked hyaluronic acid with 1.5% Extralink thiol crosslinker; “0.5% Extralink” = Chemically-crosslinked hyaluronic acid with 0.5% Extralink thiol crosslinker.

FIGS. 5A-5D are graphical representations showing homogeneity of exemplary temperature-responsive polymer combination preparations in a hydrogel state (when its precursor state was maintained at a temperature of 2-8° C. over a period of 1 month), with weekly measurements occurring at 37° C. (above CGT), as compared to control polymer compositions. Gel homogeneity was determined by measuring the storage modulus of hydrogels over a period of time. FIG. 5A: Control gel (18%w/w poloxamer 407); FIG. 5B: Temperature-responsive polymer combination preparation of 13.5% w/w poloxamer 407 and 0.65% w/w 1.5 MDa HA in 10 mM PBS at pH 7.4; FIG. 5C: Temperature-responsive polymer combination preparation of 10% w/w poloxamer 407 and 1% w/w 1.5 MDa HA in 10 mM PBS at pH 7.4; FIG. 5D: Temperature-responsive polymer combination preparation of 13.5% w/w poloxamer 407 and 0.65% w/w 1.5 MDa HA in 0.1 M bicarbonate buffer at pH 8.0.

FIGS. 6A-6B are graphical representations showing in vitro cumulative release profile of exemplary lipophilic agents from exemplary temperature-responsive polymer combination preparations in a hydrogel state at a temperature of 37° C. over a period of time. FIG. 6A: Sudan Orange; FIG. 6B: Nile Red.

FIGS. 7A-7B are graphical representations showing in vitro cumulative release profile of exemplary hydrophilic agents from exemplary temperature-responsive polymer combination preparations in a hydrogel state at a temperature of 37° C. over a period of time. FIG. 7A: Methylene Blue; FIG. 7B: Rhodamine 6G.

FIGS. 8A-8E are graphical representation showing in vivo survival data of tumor resection animals administered with exemplary temperature-responsive polymer combination preparations in a hydrogel state alone or incorporated with a TLR7/8 agonist (e.g., resiquimod), as compared to control chemically-crosslinked hyaluronic acid hydrogels alone or incorporated with a TLR7/8 agonist (e.g., resiquimod). The x-axis indicates time post-tumor inoculation. Tumor resection was performed at Day 10 post-tumor inoculation, and an exemplary composition was administered following the tumor resection. FIG. 8A: Control 12.5% (w/v) Extralink® Hyaluronic acid (HyStem®) hydrogel with or without a TLR7/8agonist (e.g., resiquimod). FIG. 8B: Temperature-responsive polymer combination preparation of 10% w/w poloxamer 407 and 1% w/w 1.5 MDa HA in 10 mM PBS at pH 7.4, with or without a TLR7/8 agonist (e.g., resiquimod). FIG. 8C: Temperature-responsive polymer combination preparation of 13.5% w/w poloxamer 407 and 0.65% w/w 1.5 MDa HA in 10 mM PBS at pH 7.4, with or without a TLR7/8 agonist (e.g., resiquimod). FIG. 8D: Temperature-responsive polymer combination preparation of 13.5% w/w poloxamer 407 and 0.65% w/w 1.5 MDa HA in 0.1 M bicarbonate buffer at pH 8.0, with or without a TLR7/8 agonist (e.g., resiquimod). FIG. 8E: Temperature-responsive polymer combination preparation of 13.5% w/w poloxamer 407 and 1.3% w/w CMCH in 10 mM PBS at pH 7.4, with or without a TLR7/8 agonist (e.g., resiquimod).

FIG. 9 shows survival data of animals receiving a liquid preparation of an immunomodulatory polymer combination preparation (e.g., a liquid preparation of a combination of carboxymethyl chitosan (CMCH) at different concentrations and a poloxamer, e.g., P407), as compared to animals receiving a liquid preparation of a poloxamer, e.g., P407 alone. The x-axis indicates time post-tumor inoculation. Tumor resection was performed at Day 10 post-tumor inoculation, and an exemplary composition was administered following the tumor resection.

FIGS. 10A-10D are graphical representations showing in vivo survival data of tumor resection animals administered with exemplary temperature-responsive polymer combination preparations (e.g., a thermoresponsive liquid preparation comprising a combination of 730 kDa or 1.5 MDa Hyaluronic Acid (HA) at different concentrations and a poloxamer, e.g., P407), as a polymer combination alone or incorporated with an immunomodulatory payload such as, e.g., a TLR7/8 agonist (e.g., resiquimod, aka R848). The x-axis indicates time post-tumor inoculation. Tumor resection was performed at Day 10 post-tumor inoculation, and an exemplary composition was administered following the tumor resection. FIG. 10A: Temperature-responsive polymer combination preparation of 10% w/w poloxamer 407 and 2.25% w/w 730 kDa HA in 12.5 mM PBS at pH 8, with or without a TLR7/8 agonist (e.g., resiquimod). FIG. 10B: Temperature-responsive polymer combination preparation of 10% w/w poloxamer 407 and 2.25% w/w 730 kDa HA in 25 mM PBS at pH 8, with or without a TLR7/8 agonist (e.g., resiquimod). FIG. 10C: Temperature-responsive polymer combination preparation of 12.5% w/w poloxamer 407 and 1.625% 730 kDa HA in 25 mM PBS at pH 8, with or without a TLR7/8 agonist (e.g., resiquimod). FIG. 10D: Temperature-responsive polymer combination preparation of 8% w/w poloxamer 407 and 2.25% w/w 730 kDa HA in 25 mM buffered saline at pH 8, with or without a TLR7/8 agonist (e.g., resiquimod).

FIG. 11 is a graphical representation showing in vivo survival data of tumor resection animals administered with exemplary temperature-responsive polymer combination preparations (e.g., a thermoresponsive liquid preparation comprising a combination of 119 kDa Hyaluronic Acid (HA) with a poloxamer, e.g., P407), as a polymer combination alone or incorporated with an immunomodulatory payload such as, e.g., a TLR7/8 agonist (e.g., resiquimod, aka R848). Shown are results from temperature-responsive polymer combination preparation of 10% w/w poloxamer 407 and 4% w/w 119 kDa HA 25 mM buffered saline pH 7.4, with or without a TLR7/8 agonist (e.g., resiquimod). The x-axis indicates time post-tumor inoculation. Tumor resection was performed at Day 10 post-tumor inoculation, and an exemplary composition was administered following the tumor resection.

FIG. 12 is a graphical representation showing in vivo survival data of tumor resection animals administered with exemplary temperature-responsive polymer combination preparations (e.g., a thermoresponsive liquid preparation comprising a combination of 309 kDa Hyaluronic Acid (HA) with a poloxamer, e.g., P407), as a polymer combination alone or incorporated with an immunomodulatory payload such as, e.g., a TLR7/8 agonist (e.g., resiquimod, aka R848), or a poloxamer only control animal cohort. Shown are results from temperature-responsive polymer combination preparations of 10% w/w poloxamer 407 and 2% w/w 309 kDa HA 25 mM buffered saline at pH 7.4, with or without a TLR7/8 agonist (e.g., resiquimod), and a control preparation of 15% poloxamer 407 biomaterial without active agent. The x-axis indicates time post-tumor inoculation. Tumor resection was performed at Day 10 post-tumor inoculation, and an exemplary composition was administered following the tumor resection.

FIG. 13 shows survival data of animals receiving a liquid preparation of exemplary immunomodulatory polymer combination preparations (e.g., a liquid preparation of a combination of low molecular weight Hyaluronic Acid (HA) and a poloxamer, e.g., P407), as compared to animals receiving a liquid preparation of a poloxamer, e.g., P407 alone. The x-axis indicates time post-tumor inoculation. Tumor resection was performed at Day 10 post-tumor inoculation, and an exemplary composition was administered following the tumor resection.

### CERTAIN DEFINITIONS

It is noted that the concentrations of individual polymer components in polymer combination preparations described herein are each expressed in % (w/w) or wt%. As used herein, the concentration,% (w/w), of a polymer component in a polymer combination preparation is determined based on the mass or weight of the polymer component relative to the sum of (i) total mass or weight of all individual polymer components present in the polymer combination preparation and (ii) total mass or weight solvent used in the polymer combination preparation.

### Activator of Adaptive Immune Response

The term “activator of adaptive immune response” refers to an agent that activates (e.g., increases the activity of) an adaptive immune system (and/or one or more features of an adaptive immune system) in a subject (e.g., in a subject to whom it is administered and/or who is otherwise in need thereof), as compared to when the agent is absent. Such activation can restore or enhance antitumor function, for example, by neutralizing inhibitory immune checkpoints and/or by triggering co-stimulatory receptors, ultimately generating helper and/or effector T cell responses against immunogenic antigens expressed by cancer cells and producing memory B cell, and/or T cell populations. In certain embodiments, an activator of adaptive immune response involves modulation of an adaptive immune response and/or leukocyte trafficking. Examples of activators of adaptive immune response include, e.g., ones described in WO 2018/045058, the contents of which are incorporated herein by reference in their entirety for the purposes described herein.

### Activator of Innate Immune Response

The term “activator of innate immune response” refers to an agent that activates (e.g., increases the activity of) an innate immune system (and/or one or more features of an innate immune system) in a subject (e.g., in a subject to whom it is administered and/or who is otherwise in need thereof), as compared to when the agent is absent. Such activation can stimulate (e.g., can increase expression level and/or activity of) one or more agents that initiate an inflammatory response (e.g., an immunostimulatory inflammatory response) and/or help to induce adaptive immune responses, for example, leading to the development of antigen-specific acquired immunity. In some embodiments, activation of the innate immune system can lead to recruitment of relevant immune cells including, e.g., but not limited to neutrophils, basophils, eosinophils, natural killer cells, dendritic cells, monocytes, and macrophages, cytokine production, leukocyte proliferation and/or survival, as well as improved T cell priming, for example by augmenting presentation of antigens and/or expression level and/or activity of co-stimulatory molecules by antigen-presenting cells. Examples of activators of innate immune response include, e.g., ones described in WO 2018/045058, the contents of which are incorporated herein by reference in their entirety for the purposes described herein.

### Administer

As used herein, the term “administer,” “administering,” or “administration” typically refers to the administration of a composition to a subject to achieve delivery of an agent or payload that is, or is included in, a composition to a target site or a site to be treated. Those of ordinary skill in the art will be aware of a variety of routes that may, in appropriate circumstances, be utilized for administration of different agents to a subject, for example a human. For example, while the terms “administer,” “administering,” or “administration” refer to implanting, absorbing, ingesting, injecting, inhaling, parenteral administration, or otherwise introducing a composition as described herein, in the context of administering a composition comprising a provided polymer combination preparation (with or without a payload incorporated therein), administering may refer to, in some embodiments, implanting, or in some embodiments, injecting.

### Agonist

Those skilled in the art will appreciate that the term “agonist” may be used to refer to an agent, condition, or event whose presence, level, degree, type, or form correlates with increased level and/or activity of another agent (i.e., the agonized agent) and/or an increase in or induction of one or more biological events. In general, an agonist may be or include an agent of various chemical class including, for example, small molecules, polypeptides, nucleic acids, carbohydrates, lipids, metals, inorganic crystals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

### Antagonist

Those skilled in the art will appreciate that the term “antagonist” may refer to an agent, condition, or event whose presence, level, degree, type, or form is associated with a decreased level and/or activity of another agent (i.e., the antagonized agent) and/or a decrease in or suppression of one or more biological events. In general, an antagonist may include an agent of various chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered).

### Antibody

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y’s stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains - an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi- specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies. etc. In some embodiments, relevant formats may be or include: Adnectins®; Affibodies®; Affilins®; Anticalins®; Avimers®; BiTE®s; cameloid antibodies; Centyrins®; ankyrin repeat proteins or DARPINs®; dual-affinity re-targeting (DART) agents; Fynomers®; shark single domain antibodies such as IgNAR; immune mobilizing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs®”); single chain or Tandem diabodies (TandAb®); TCR-like antibodies;, Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

### Bioadhesive

The term “bioadhesive” refers to a biocompatible agent that can adhere to a target surface, e.g., a tissue surface. In some embodiments, a bioadhesive can adhere to a target surface, e.g., a tissue surface, and retain on the target surface, e.g., for a period of time. In some embodiments, a bioadhesive may be biodegradable. In some embodiments, a bioadhesive may be a natural agent, which may have been prepared or obtained, for example, by isolation or by synthesis; in some embodiments, a bioadhesive may be a non-natural agent, e.g., as may have been designed and/or manufactured by the hand of man (e.g., by processing, synthetic, and/or recombinant production, depending on the agent, as will be understood by those skilled in the art. In some particular embodiments, a bioadhesive may be or comprise a polymeric material, e.g., as may be comprised of or contain a plurality of monomers such as sugars. Certain exemplary bioadhesives include a variety of FDA-approved agents such as, for example, cyanoacrylates (Dermabond, 2-Octyl cyanoacrylate; Indermil, n-Butyl-2-cyanoacrylate; Histoacryl and Histoacryl Blue, n-Butyl-2-cyanoacrylate), albumin and glutaraldehyde (BioGlue®, bovine serum albumin and 10% glutaraldehyde), fibrin glue (Tisseel®, human pooled plasma fibrinogen and thrombin; Evicel®, human pooled plasma fibrinogen and thrombin; Vitagel®, autologous plasma fibrinogen and thrombin; Cryoseal®system, autologous plasma fibrinogen and thrombin), gelatin and/or resorcinol crosslinked by formaldehyde and/or glutaraldehyde, polysaccharide-based adhesives (e.g., alginate, chitosan, collagen, dextran, and/or gelatin), PEG, acrylates, polyamines, or urethane variants (isocyanate-terminated prepolymer, and/or combinations thereof. Other examples of bioadhesives that are known in the art, e.g., as described in Mehdizadeh and Yang “Design Strategies and Applications of Tissue Bioadhesives” Macromol Biosci 13:271-288 (2013), can be used for the purposes of the methods described herein. In some embodiments, a bioadhesive can be a degradable bioadhesive. Examples of such a degradable bioadhesive include, but are not limited to fibrin glues, gelatin-resorcinol-formaldehyde/glutaraldehyde glues, poly(ethylene glycol) (PEG)-based hydrogel adhesives, polysaccharide adhesives, polypeptide adhesives, polymeric adhesives, biomimetic bioadhesives, and ones described in Bhagat and Becker “Degradable Adhesives for Surgery and Tissue Engineering” Biomacromolecules 18: 3009-3039 (2017).

### Biocompatible

The term “biocompatible”, as used herein, refers to materials that do not cause significant harm to living tissue when placed in contact with such tissue, e.g., in vivo. Biocompatibility of a material can be gauged by the ability of such a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material’s toxicity, infectivity, pyrogenicity, irritation potential, reactivity, hemolytic activity, carcinogenicity, and/or immunogenicity. In certain embodiments, materials are “biocompatible” if they themselves are not toxic to cells in an in vivo environment of its intended use. In certain embodiments, materials are “biocompatible” if their addition to cells in vitro results in less than or equal to 20% cell death and/or their administration in vivo does not induce significantly severe inflammation that is clinically undesirable for purposes described herein or other such adverse effects. As will be understood by those skilled in the art that such significantly severe inflammation is distinguishable from mild, transient inflammation, which typically accompanies surgery or introduction of foreign objects into a living organism. Furthermore, one of skill in the art will appreciate, reading the present disclosure, that in some embodiments, polymer combination preparations described herein and/or individual polymer components thereof are biocompatible if extent of immunomodulation (e.g., innate immunity agonism) over a defined period of time is clinically beneficial and/or desirable, e.g., to provide antitumor immunity.

### Biodegradable

As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significantly severe inflammation that is clinically undesirable for purposes described herein and/or other adverse effects in vivo. In some embodiments, biodegradable polymer materials break down into their component monomers. In some embodiments, biodegradable polymer materials may be biologically degraded, e.g., by enzymatic activity or cellular machinery, in some cases, for example, through exposure to a lysozyme (e.g., having relatively low pH), or by simple hydrolysis. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates), poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose variants and polyhydroxyalkanoates, for example, polyhydroxybutyrate blends and copolymers thereof. Those of ordinary skill in the art will appreciate or be able to determine when such polymers are biocompatible and/or biodegradable variants thereof (e.g., related to a parent polymer by substantially identical structure that differs only in substitution or addition of particular chemical groups as is known in the art).

### Biologic

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

### Biological Sample

The term “biological sample” refers to a primary sample obtained from a biological source and/or, in some embodiments, to a sample derived therefrom (e.g., by processing). Those skilled in the art appreciate that biological samples may include or be selected from, for example, tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

### Biomaterial

The term “biomaterial” refers to a biocompatible substance characterized in that it can be administered to a subject for a medical purpose (e.g., therapeutic, diagnostic) without eliciting an unacceptable (according to sound medical judgement) reaction. Biomaterials can be obtained or derived from nature or synthesized. In some embodiments, a biomaterial may be or comprise a polymeric biomaterial. For example, in some embodiments, a polymeric biomaterial may comprise at least one or a plurality of (e.g., at least two or more) polymer components. In some embodiments, a biomaterial can be in a form of a polymer network. In some embodiments, a biomaterial can be in an injectable format, e.g., a viscous solution. For example, a biomaterial can comprise its precursor components to be formed in situ (e.g., upon administration to a subject). In some embodiments, a biomaterial can be a liquid. In some embodiments, a biomaterial is a viscous solution. In some embodiments, a biomaterial is a colloid. In some embodiments, a biomaterial can be a solid. In some embodiments, a biomaterial can be a crystal (e.g., an inorganic crystal). In some embodiments, a biomaterial is not a nucleic acid. In some embodiments, a biomaterial is not a polypeptide.

### Cancer

The term “cancer” refers to a malignant neoplasm (Stedman’s Medical Dictionary, 25th ed.; Hensyl ed.; Williams & Wilkins: Philadelphia, 1990). Of particular interest in the context of some embodiments of the present disclosure are cancers treated by cell killing and/or removal therapies (e.g., surgical resection and/or certain chemotherapeutic therapies such as cytotoxic therapies, etc.). In some embodiments, a cancer that is treated in accordance with the present disclosure is one that has been surgically resected (i.e., for which at least one tumor has been surgically resected). In some embodiments, a cancer that is treated in accordance with the present disclosure is one for which resection is standard of care. In some embodiments, a cancer that is treated in accordance with the present disclosure is one that has metastasized. In certain embodiments, exemplary cancers may include one or more of acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi’s sarcoma, multiple idiopathic hemorrhagic sarcoma); endometrial cancer (e.g., uterine cancer, uterine sarcoma); esophageal cancer (e.g., adenocarcinoma of the esophagus, Barrett’s adenocarcinoma); Ewing’s sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström’s macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget’s disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget’s disease of the vulva).

### Carbohydrate Polymer

The term “carbohydrate polymer” refers to a polymer that is or comprises one or more carbohydrates, e.g., having a carbohydrate backbone. For example, in some embodiments, a carbohydrate polymer refers to a polysaccharide or an oligosaccharide, or a polymer containing a plurality of monosaccharide units connected by covalent bonds. The monosaccharide units may all be identical, or, in some cases, there may be more than one type of monosaccharide unit present within the carbohydrate polymer. In certain embodiments, a carbohydrate polymer is naturally occurring. In certain embodiments, a carbohydrate polymer is synthetic (i.e., not naturally occurring). In some embodiments, a carbohydrate polymer may comprise a chemical modification. In some embodiments, a carbohydrate polymer is a linear polymer. In some embodiments, a carbohydrate polymer is a branched polymer.

### Chemotherapeutic Agent

The term “chemotherapeutic agent” refers to a therapeutic agent known to be of use in chemotherapy for cancer. For example, in some embodiments, a chemotherapeutic agent can inhibit the proliferation of rapidly growing cancer cells and/or kill cancer cells. Examples of such chemotherapeutic agents include, but are not limited to alkylating agents, anti-metabolites, topoisomerase inhibitors, and/or mitotic inhibitors.

### Combination Therapy

As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

### Colloid

As used herein, the term “colloid” refers to a homogenous solution or suspension of particles (e.g., polymer particles) dispersed though a continuous medium (e.g., an aqueous buffer system). In some embodiments, a colloid is an emulsion. In some embodiments, a colloid is a sol. In some embodiments, a colloid is a gel.

### Comparable

As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those of ordinary skill in the art will also understand that when the term “comparable” is used in the context of comparison of two or more values, such values are comparable to one another such that the differences in values do not result in material differences in therapeutic outcomes, e.g., induction of anti-tumor immunity and/or incidence of tumor regrowth and/or metastasis. For example, in some embodiments, comparable release rates refer to values of such release rates within 15% over a period of 48 hours. In some embodiments, comparable release rates refer to values of such release rates within 20% over a period of 48 hours. In some embodiments, comparable release rates refer to values of such release rates within 15% over a period of 24 hours.

### Condition, Disease, or Disorder

The terms “condition,” “disease,” and “disorder” are used interchangeably.

### Corresponding To

As used herein, the term “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

### Critical Gelation Temperature

As used herein, the term “critical gelation temperature”, abbreviated as “CGT”, refers to a threshold temperature at or above which a precursor state of a polymer combination preparation (e.g., ones described herein) transitions to a polymer network state described herein (e.g., a hydrogel state). In some embodiments, a critical gelation temperature may correspond to a sol-gel transition temperature. In some embodiments, a critical gelation temperature may correspond to a lower critical solution temperature. See Taylor et al., “Thermoresponsive Gels” Gels (2017) 3:4, for general description of thermoresponsive gels, the contents of which are incorporated herein by reference for purposes described herein. As described in the present disclosure, certain embodiments of polymer combination preparations described herein are demonstrated to form a polymer network state when it is exposed to a temperature of about 35-40° C. One of ordinary skill in the art, reading the present disclosure, will understand that such polymer combination preparations do not necessarily have a CGT of about 35-40° C., but may rather have a CGT that is lower than 35-40° C. For example, in some embodiments, provided polymer combination preparations may have a CGT of about 20-28° C.

### Critical Gelation Weight Ratio

As used herein, the term “critical gelation weight ratio” refers to a threshold weight ratio of at least two or more polymer components in a provided polymer combination preparation, at or above which a precursor state of such a polymer combination preparation (e.g., ones described herein) transitions to a polymer network state described herein (e.g., a hydrogel state). In some embodiments, such a precursor-polymer network transition occurs when both a critical gelation temperature and a critical gelation weight ratio for a provided polymer combination preparation are achieved.

### Crosslink

As used herein, the term “crosslink” refers to interaction and/or linkage between one entity and another entity to form a network. For example, in some embodiments, crosslinks present in polymer network may be or comprise intra-molecular crosslinks, intermolecular crosslinks, or both. In some embodiments, crosslinks may comprise interactions and/or linkages between one polymer chain(s) and another polymer chain(s) to form a polymer network. In some embodiments, a crosslink may be achieved using one or more physical crosslinking approaches, including, e.g., one or more environmental triggers and/or physiochemical interactions. Examples of an environmental trigger include, but are not limited to pH, temperature, and/or ionic strength. Non-limiting examples of physiochemical interactions include hydrophobic interactions, charge interactions, hydrogen bonding interactions, stereocomplexation, and/or supramolecular chemistry. In some embodiments, a crosslink may be achieved using one or more covalent crosslinking approaches (e.g., where the linkage between two entities is or comprises a covalent bond) based on chemistry reactions, e.g., in some embodiments which may include reaction of an aldehyde and an amine to form a Schiff base, reaction of an aldehyde and hydrazide to form a hydrazine, and/or Michael reaction of an acrylate and either a primary amine or a thiol to form a secondary amine or a sulfide. Examples of such covalent crosslinking approaches include, but are not limited to small-molecule crosslinking and polymer-polymer crosslinking. Various methods for physical and covalent crosslinking of polymer chains are known in the art, for example, as described in Hoare and Kohane, “Hydrogels in drug delivery: Progress and challenges” Polymer (2008) 49:1993-2007, the entire content of which is incorporated herein by reference for the purposes disclosed herein.

### Crosslinker

As used interchangeably herein, the term “crosslinker” or “crosslinking agent” refers to an agent that links one entity (e.g., one polymer chain) to another entity (e.g., another polymer chain). In some embodiments, linkage (i.e., the “crosslink”) between two entities is or comprises a covalent bond. In some embodiments, linkage between two entities is or comprises an ionic bond or interaction. In some embodiments, a crosslinker is a chemical crosslinker, which, e.g., in some embodiments may be or comprise a small molecule (e.g., dialdehydes or genipin) for inducing formation of a covalent bond between an aldehyde and an amino group. In some embodiments, a crosslinker comprises a photo-sensitive functional group. In some embodiments, a crosslinker comprises a pH-sensitive functional group. In some embodiments, a crosslinker comprises a thermal-sensitive functional group.

### Disease

As used herein, the term “disease” refers to a disorder or condition that typically impairs normal functioning of a tissue or system in a subject (e.g., a human subject) and is typically manifested by characteristic signs and/or symptoms. Examples of diseases that are amenable for technologies provided herein include, but are not limited to autoimmune diseases, inflammatory diseases, bone diseases, metabolic diseases, neurological and neurodegenerative diseases, cancer, cardiovascular diseases, allergies and asthma, Alzheimer’s disease, and hormone-related diseases. In some embodiments, a disease amenable to technologies provided herein is cancer.

### Effective Amount

An “effective amount” is an amount sufficient to elicit a desired biological response, e.g., treating a condition from which a subject may be suffering. As will be appreciated by those of ordinary skill in this art, the effective amount of a composition or an agent included in the composition may vary depending on such factors as the desired biological endpoint, the physical, chemical, and/or biological characteristics (e.g., pharmacokinetics and/or degradation) of agents in the composition, the condition being treated, and the age and health of the subject. In some embodiments, an amount may be effective for therapeutic treatment; alternatively or additionally, in some embodiments, an amount may be effective for prophylactic treatment. For example, in treating cancer, an effective amount may prevent tumor regrowth, reduce the tumor burden, or stop the growth or spread of a tumor. Those skilled in the art will appreciate that an effective amount need not be contained in a single dosage form. Rather, administration of an effective amount may involve administration of a plurality of doses, potentially over time (e.g., according to a dosing regimen). For example, in some embodiments, an effective amount may be an amount administered in a dosing regimen that has been established, when administered to a relevant population, to achieve a particular result with statistical significance.

### Hydrate

The term “hydrate”, as used herein, has its art-understood meaning and refers to an aggregate of a compound (which may, for example be a salt form of the compound) and one or more water molecules. Typically, the number of the water molecules contained in a hydrate of a compound is in a definite ratio to the number of the compound molecules in the hydrate. Therefore, a hydrate of a compound may be represented, for example, by the general formula R×x H2O, wherein R is the compound and x is a number greater than 0. A given compound may form more than one type of hydrate, including, e.g., monohydrates (x is 1), lower hydrates (x is a number greater than 0 and smaller than 1, e.g., hemihydrates (Rx0.5 H2O)), and polyhydrates (x is a number greater than 1, e.g., dihydrates (Rx2 H2O) and hexahydrates (Rx6 H2O)).

### Hydrogel

The term “hydrogel” has its art-understood meaning and refers to a material formed from a network of polymer chains that are hydrophilic, sometimes found as a colloidal gel in which an aqueous phase is the dispersion medium. In some embodiments, hydrogels are highly absorbent (e.g., they can absorb and/or retain over 90% water) natural or synthetic polymeric networks. In some embodiments, hydrogels possess a degree of flexibility similar to natural tissue, for example due to their significant water content.

### Immunotherapy

The term “immunotherapy” refers to a therapeutic agent that promotes the treatment of a disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, nucleic acids, small molecule inhibitors, and small molecule agonists. For example, useful immunotherapies may include, but are not limited to, inducers of type I interferon, interferons, stimulator of interferon genes (STING) agonists, TLR7/8 agonists, IL-15 superagonists, COX inhibitors (e.g., COX-1 inhibitors and/or COX-2 inhibitors), anti-PD-1 antibodies, anti-CD137 antibodies, and anti-CTLA-4 antibodies. In some embodiments, certain polymer combination preparations provided herein are themselves immunomodulatory (e.g., sufficient to induce anti-tumor immunity) in the absence of immunotherapy and thus do not include administration of such immunotherapy as described herein.

### Immunomodulatory Payload

As used herein, the term “immunomodulatory payload” refers to a separate immunomodulatory agent (e.g., small molecules, polypeptides (including, e.g., cytokines), nucleic acids, etc.) that can be carried by or distributed in a polymer combination preparation such as ones as provided and/or utilized herein), wherein the immunomodulatory agent provides a therapeutic effect of modulating or altering (e.g., inducing, enhancing, or suppressing, etc.) one or more aspects of an immune response in a subject. Examples of an immunomodulatory payload include, but are not limited to activators of adaptive immune response, activators of innate immune response, inhibitors of a proinflammatory pathway, immunomodulatory cytokines, or immunomodulatory therapeutic agents as well as ones as described in WO 2018/045058 and WO 2019/183216, and any combinations thereof. The contents of the aforementioned patent application are incorporated herein by reference for the purposes described herein. In some embodiments, an immunomodulatory payload is or comprises an innate immunity modulatory payload (e.g., an immunomodulatory payload that induces or stimulates innate immunity and/or one or more features of innate immunity). In some embodiments, an innate immunity modulatory payload is or comprises an activator of innate immune response. In some embodiments, an immunomodulatory payload is or comprises an adaptive immunity modulatory payload, e.g., an activator of adaptive immune response. In some embodiments, an immunomodulatory payload is or comprises an inhibitor of a proinflammatory pathway, e.g., an inhibitor of proinflammatory immune response mediated by a p38 mitogen-activated protein kinase (MAPK) pathway. In some embodiments, an immunomodulatory payload is or comprises an immunomodulatory cytokine. In some embodiments, an immunomodulatory payload is or comprises an immunomodulatory therapeutic agent. As will be understood by those skilled in the art, an immunomodulatory payload does not include components (e.g., precursor components) and/or by-products of a polymer combination preparation (e.g., as described and/or utilized herein) generated, e.g., by chemical, enzymatic, and/or biological reactions such as, e.g., degradation.

### Implanting

The terms “implantable,” “implantation,” “implanting,” and “implant” refer to positioning a composition of interest at a specific location in a subject, such as within a tumor resection site or in a sentinel lymph node, and typically by general surgical methods.

### Increased, Induced, or Reduced

As used herein, these terms or grammatically comparable comparative terms, indicate values that are relative to a comparable reference measurement. For example, in some embodiments, an assessed value achieved with a provided polymer combination preparation (e.g., in a precursor state or in a polymer network state) may be “increased” relative to that obtained with a comparable reference biomaterial preparation (e.g., a biomaterial of 18% (w/w) Poloxamer 407, or a chemically-crosslinked hydrogel such as, e.g., a chemically crosslinked hyaluronic acid hydrogel). Alternatively or additionally, in some embodiments, an assessed value achieved in a subject may be “increased” relative to that obtained in the same subject under different conditions (e.g., prior to or after an event; or presence or absence of an event such as administration of a composition or preparation as described and/or utilized herein, or in a different, comparable subject (e.g., in a comparable subject that differs from the subject of interest in prior exposure to a condition, e.g., absence of administration of a composition or preparation as described and/or utilized herein.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

### Inhibit

The term “inhibit” or “inhibition” is not limited to only total inhibition. Thus, in some embodiments, partial inhibition or relative reduction is included within the scope of the term “inhibition.” For example, in the context of modulating level (e.g., expression and/or activity) of a target, the term, in some embodiments, refers to a reduction of the level (e.g., expression and/or activity) of a target to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target. In the context of risk and/or incidence of tumor recurrence and/or metastasis, the term, in some embodiments, refers to a reduction of the risk or incidence of tumor recurrence and/or metastasis to a level that is reproducibly and/or statistically significantly lower than an initial or other appropriate reference level, which may, for example, be a baseline level of risk or incidence of tumor recurrence and/or metastasis in the absence or prior to administration of a composition described herein. In some embodiments, the term refers to a reduction of the risk or incidence of tumor recurrence and/or metastasis to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of risk or incidence of tumor recurrence and/or metastasis in the absence or prior to administration of a composition described herein.

### Inhibitor

As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

### Inhibitor of a Proinflammatory Pathway

The term “inhibitor of a proinflammatory pathway” as used herein, in some embodiments, refers to an agent that inhibits or reduces inflammation that is associated with immunosuppression. In some embodiments, such an inhibitor of a proinflammatory pathway refers to an agent that prevents recruitment of immunosuppressive cells or prevents acute inflammation. Such acute inflammation and/or recruitment of immunosuppressive cells can occur after local trauma, including that which is caused by surgery. In some embodiments, an inhibitor of a proinflammatory pathway may inhibit, for example, an immune response that induces inflammation, including, e.g., production of inflammatory cytokines (including, e.g., but not limited to TGF-β and IL-10), increased activity and/or proliferation of M2-like macrophages, recruitment of relevant immune cells including, e.g., but not limited to myeloid cells, neutrophils, and mast cells, etc. Examples of inhibitors of a proinflammatory pathway include, e.g., ones described in International Application Number WO 2019/183216, the contents of which are incorporated herein by reference in their entirety for the purposes described herein.

### Isomers

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

### Lymph Node

As is known in the art, the term “lymph node” refers to components of the lymphatic system that are small structures, located throughout the body, through which lymph fluid flows. Lymph nodes are understood to filter certain substances from lymphatic fluid. Lymph nodes also can contain immune cells, for example that may participate in immune reactions throughout the body. In some embodiments, a lymph node may be or comprise a sentinel lymph node (i.e., a lymph node to which cancer cells are most likely to spread from a primary tumor).

### Marker

A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

### Metastasis

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

### Microparticle

As used herein, the term “microparticle” refers to a particle having a longest dimension (e.g., a diameter) between 1 micrometer and 1,000 micrometers (µm). In some embodiments, a microparticle may be characterized by a longest dimension (e.g., a diameter) of between 1 µm and 500 µm. In some embodiments, a microparticle may be characterized by a longest dimension (e.g., a diameter) of between 1 µm and 100 µm. In many embodiments, a population of microparticles is characterized by an average size (e.g., longest dimension) that is below about 1,000 µm, about 500 µm, about 100 µm, about 50 µm, about 40 µm, about 30 µm, about 20 µm, or about 10 µm and often above about 1 µm. In many embodiments, a microparticle may be substantially spherical (e.g., so that its longest dimension may be its diameter.

### Monosaccharide

As used herein, the term “monosaccharide” is given its ordinary meaning as used in the art and refers to a simple form of a sugar that consists of a single saccharide unit which cannot be further decomposed to smaller saccharide building blocks or moieties. Common examples of monosaccharides include, e.g., glucose (dextrose), fructose, galactose, mannose, ribose, etc. Monosaccharides can be classified according to the number of carbon atoms of the carbohydrate, for example, triose, having 3 carbon atoms such as glyceraldehyde and/or dihydroxyacetone; tetrose, having 4 carbon atoms such as erythrose, threose and/or erythrulose; pentose, having 5 carbon atoms such as arabinose, lyxose, ribose, xylose, ribulose and/or xylulose; hexose, having 6 carbon atoms such as allose, altrose, galactose, glucose, gulose, idose, mannose, talose, fructose, psicose, sorbose and/or tagatose; heptose, having 7 carbon atoms such as mannoheptulose, and/or sedoheptulose; octose, having 8 carbon atoms such as 2-keto-3-deoxy-manno-octonate; nonose, having 9 carbon atoms such as sialose; and decose, having 10 carbon atoms. The above monosaccharides encompass both D-and L-monosaccharides. Alternatively, a monosaccharide can be a monosaccharide variant, in which the saccharide unit comprises one or more substituents (e.g., deoxy, H substituents, heteroatom substituents (e.g., S, Cl, F, etc.), etc.) other than a hydroxyl. Such variants can be, but are not limited to, ethers, esters, amides, acids, phosphates and amines. Amine variants (i.e., amino sugars) include, for example, glucosamine, galactosamine, fructosamine and/or mannosamine. Amide variants include, for example, N-acetylated amine variants of saccharides (e.g., N-acetylglucosamine, and/or N-acetylgalactosamine).

### Modulator

As used herein, the term “modulator” may be or comprise an entity whose presence or level in a system in which an activity of interest is observed correlates with a change in level and/or nature of that activity as compared with that observed under otherwise comparable conditions when the modulator is absent. In some embodiments, a modulator is an activator or agonist, in that an activity of interest is increased in its presence as compared with that observed under otherwise comparable conditions when the modulator is absent. In some embodiments, a modulator is an antagonist or inhibitor, in that an activity of interest is reduced in its presence as compared with otherwise comparable conditions when the modulator is absent. In some embodiments, a modulator interacts directly with a target entity whose activity is of interest. In some embodiments, a modulator interacts indirectly (e.g., interacts with one or more entities that interacts and/or are associated with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

### Modulator of Macrophage Effector Function

The term “modulator of macrophage effector function” refers to an agent that activates macrophage effector function or depletes immunosuppressive macrophages or macrophage-derived suppressor cells. Such potentiation can mobilize macrophage and myeloid components to destroy the tumor and its stroma, including the tumor vasculature. Macrophages can be induced to secrete antitumor cytokines and/or to perform phagocytosis, including antibody-dependent cellular phagocytosis.

### Modulator of Neutrophil Function

As used interchangeably herein, the terms “modulator of neutrophils” and “modulator of neutrophil function” refer to a modulator of one or more biological functions and/or phenotypes of neutrophils. For example, in some embodiments, a modulator of neutrophil function can inhibit recruitment, survival, and/or proliferation of neutrophils. Additionally or alternatively, in some embodiments, a modulator of neutrophil function can modulate neutrophil-associated effector function, which may include but are not limited to, modulation of production and/or secretion of one or more immunomodulatory molecules (e.g., immunomodulatory cytokines and/or chemokines) and/or alter extracellular-matrix modifying capabilities of neutrophils. In some embodiments, a modulator of neutrophil function (e.g., ones described herein) may act on or target neutrophils only. In some embodiments, a modulator of neutrophil function (e.g., ones described herein) may act on neutrophils and at least one additional type of immune cells, e.g., other subsets of myeloid-derived suppressive cells (MDSCs), macrophages, and/or monocytes. One of ordinary skill in the art will appreciate that at least a subset of neutrophils may exhibit similar immune activities as one or more certain subsets of MDSCs and thus be considered as polymorphonuclear and/or granulocytic MDSCs (for example, as described in: Mehmeti-Ajradini et al., “Human G-MDSCs are neutrophils at distinct maturation stages promoting tumor growth in breast cancer” Life Science Alliance, Sep. 21, 2020; and Brandau et al., “A subset of mature neutrophils contains the strongest PMN-MDSC activity in blood and tissue of patients with head and neck cancer” The Journal of Immunology, May 1, 2020; the contents of each of which are incorporated herein by reference for purposes described herein).

### Nanoparticle

As used herein, the term “nanoparticle” refers to a particle having a longest dimension (e.g., a diameter) of less than 1,000 nanometers (nm). In some embodiments, a nanoparticle may be characterized by a longest dimension (e.g., a diameter) of less than 300 nm. In some embodiments, a nanoparticle may be characterized by a longest dimension (e.g., a diameter) of less than 100 nm. In many embodiments, a nanoparticle may be characterized by a longest dimension between about 1 nm and about 100 nm, or between about 1 µm and about 500 nm, or between about 1 nm and 1,000 nm. In many embodiments, a population of nanoparticles is characterized by an average size (e.g., longest dimension) that is below about 1,000 nm, about 500 nm, about 100 nm, about 50 nm, about 40 nm, about 30 nm, about 20 nm, or about 10 nm and often above about 1 nm. In many embodiments, a nanoparticle may be substantially spherical so that its longest dimension may be its diameter. In some embodiments, a nanoparticle has a diameter of less than 100 nm as defined by the National Institutes of Health.

### Neoplasm and Tumor

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor’s neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

### Payload

In general, the term “payload”, as used herein, refers to an agent that may be incorporated into a polymer combination preparation described herein. In some embodiments, a payload may refer to a compound, molecule, or entity of any chemical class including, for example, a small molecule, a peptide, a polypeptide, a nucleic acid, a saccharide (e.g., a polysaccharide), a lipid, a metal, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, a mineral, a growth factor, a cytokine, an antibody, a hormone, an extracellular matrix protein (such as collagen, vitronectin, fibrin, etc.), an extracellular matrix sugar, a chemoattractant, a polynucleotide (e.g., DNA, RNA, antisense molecule, plasmid, etc.), a microorganism (e.g., a virus), etc, or a combination thereof. In some embodiments, a payload is or comprises a therapeutic agent. Examples of a therapeutic agent include but are not limited to analgesics, antibiotics, antibodies, anticoagulants, antiemetics, cells, coagulants, cytokines, growth factors, hormones, immunomodulatory agents, polynucleotides (e.g., DNA, RNA, antisense molecules, plasmids, etc.), and combinations thereof. In some embodiments, a payload may be or comprise a cell or organism, or a fraction, extract, or component thereof. In some embodiments, a payload may be or comprise a natural product in that it is found in and/or is obtained from nature. Alternatively or additionally, in some embodiments, the term may be used to refer to one or more entities that is man-made in that it is designed, engineered, and/or produced through action of the hand of man and/or is not found in nature. In some embodiments, a payload may be or comprise an agent in isolated or pure form; in some embodiments, such an agent may be in crude form.

### Pharmaceutically Acceptable Salt

The term “pharmaceutically acceptable salt” refers to those salts which are, within the scope of sound medical judgment, suitable for use in contact with the tissues of, for example, humans and/or animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, the contents of which are incorporated herein by reference for purposes described herein. Pharmaceutically acceptable salts that may be utilized in accordance with certain embodiments of the present disclosure may include, for example, those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁-C₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Poloxamer

As used herein, the term “poloxamer” refers to a polymer preparation of or comprising one or more poloxamers. In some embodiments, poloxamers in a polymer preparation may be unconjugated or unmodified, for example, which are typically triblock copolymers comprising a hydrophobic chain of polyoxypropylene (polypropylene glycol, PPG) flanked by two hydrophilic chains of polyoxyethylene (polyethylene glycol, PEG). In some embodiments, a polymer preparation of or comprising one or more poloxamer may be unfiltered (e.g., such a polymer preparation may contain impurities and/or relatively low molecular weight polymeric molecules, as compared to a comparable polymer preparation that is filtered). Examples of poloxamers include are not limited to, Poloxamer 124 (P124, also known as Pluronic L44 NF), Poloxamer 188 (P188, also known as Pluronic F68NF), Poloxamer 237 (P237, also known as Pluronic F 87 NF), Poloxamer 338 (P338, also known as Pluronic F108 NF), Poloxamer 407 (P407, also known as Pluronic F127 NF), and combinations thereof.

### Polymer

The term “polymer” is given its ordinary meaning as used in the art, i.e., a molecular structure comprising one or more repeat units (monomers), connected by covalent bonds. The repeat units may all be identical, or, in some cases, there may be more than one type of repeat unit present within the polymer (e.g., in a copolymer). In certain embodiments, a polymer is naturally occurring. In certain embodiments, a polymer is synthetic (i.e., not naturally occurring). In some embodiments, a polymer is a linear polymer. In some embodiments, a polymer is a branched polymer. In some embodiments, a polymer for use in accordance with the present disclosure is not a polypeptide. In some embodiments, a polymer for use in accordance with the present disclosure is not a nucleic acid.

### Polymer Combination Preparation

As used herein, the term “polymer combination preparation” refers to a polymeric biomaterial comprising at least two distinct polymer components. For example, in many embodiments, a polymer combination preparation described herein is a polymeric biomaterial comprising a first polymer component and a second first polymer component, wherein the first polymer component is or comprises at least one poloxamer, and the second polymer component is or comprises a polymer that is not poloxamer. In some embodiments, a polymer combination preparation described herein is a polymeric biomaterial in a precursor state, which may be, e.g., useful for administration to a subject. In some embodiments, a polymer combination preparation described herein is a polymeric biomaterial in a polymer network state.

### Polymeric Biomaterial

A “polymeric biomaterial”, as described herein, is a material that is or comprises at least one polymer or at least one polymeric moiety and is biocompatible. In many embodiments, a polymeric biomaterial is or includes at least one polymer; in some embodiments, a polymer may be or comprise a copolymer. In some embodiments, a polymeric biomaterial is or comprises a preparation of at least two distinct polymer components (e.g., a preparation containing poloxamer and a second polymer component that is not a poloxamer). Those skilled in the art will be aware that certain polymers may exist and/or be available in a variety of forms (e.g., length, molecular weight, charge, topography, surface chemistry, degree and/or type of modification such as alkylation, acylation, quaternization, hydroxyalkylation, carboxyalkylation, thiolation, phosphorylation, glycosylation, etc.); in some embodiments, a preparation of such polymers may include a specified level and/or distribution of such form or forms. Additionally or alternatively, those skilled in the art will appreciate that, in some embodiments, one or more immunomodulatory properties of a polymeric biomaterial may be tuned by its biomaterial property(ies), including, e.g., surface chemistry of a polymeric biomaterial (e.g., modulated by hydrophobicity and/or hydrophilicity portions of a polymeric biomaterial, chemical moieties, and/or charge characteristics) and/or topography of a polymeric biomaterial (e.g., modulated by size, shape, and/or surface texture), for example as described in Mariani et al. “Biomaterials: Foreign Bodies or Tuners for the Immune Response?” International Journal of Molecular Sciences, 2019, 20, 636.

### Polymer Network

The term “polymer network” is used herein to describe an assembly of polymer chains interacting with each other. In some embodiments, a polymer network forms a three-dimensional structure material. In some embodiments, a polymer network may be formed by linking polymer chains (“crosslinked polymer network”) using a crosslinker (e.g., as described herein). In some embodiments, a polymer network is transitioned from a precursor state when it is exposed to a temperature that is or above a critical gelation temperature, wherein the polymer network state has a viscosity materially above (e.g., at least 50% or above) that of the precursor state and the polymer network state comprises crosslinks not present in the precursor state. In some embodiments, a polymer network may be formed by non-covalent or non-ionic intermolecular association of polymer chains, e.g., through hydrogen bonding. In some embodiments, a polymer network may be formed by a combination of chemically crosslinking polymer chains and non-covalent or non-ionic intermolecular association of polymer chains.

### Prodrug

The term “prodrug” refers to a form of an active compound that includes one or more cleavable group(s) that is/are removed by solvolysis or under physiological conditions, so that the active compound is released. Exemplary prodrug forms include, but are not limited to, choline ester derivatives and the like as well as N-alkylmorpholine esters and the like. In some embodiments, a prodrug may be an acid derivative, such as is known in the art, such as, for example, esters prepared by reaction of the parent acid with a suitable alcohol, amides prepared by reaction of the parent acid compound with a substituted or unsubstituted amine, acid anhydrides, or mixed anhydrides. Simple aliphatic or aromatic esters, amides, and anhydrides derived from acidic groups pendant on a compound of interest are particular examples of prodrug forms. In some cases, it may be desirable to prepare double ester-type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of a compound of interest.

### Proinflammatory Cytokine

As used herein, the term “proinflammatory cytokine” refers to a protein or glycoprotein molecule secreted by a cell (e.g., a cell of an immune system) that induces an inflammatory response. As will be appreciated by one of skilled in the art, inflammation may be immunostimulatory or immunosuppressive depending on the biological context.

### Proinflammatory Immune Response

The term “proinflammatory immune response” as used herein refers to an immune response that induces inflammation, including, e.g., production of proinflammatory cytokines (including, e.g., but not limited to CXCL10, IFN-α, IFN-β, IL-1β, IL-6, IL-18, and/or TNF-alpha), increased activity and/or proliferation of Th1 cells, recruitment of myeloid cells, etc. In some embodiments, a proinflammatory immune response may be or comprise one or both of acute inflammation and chronic inflammation.

### Proliferative Disease

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis or diseases associated with angiogenesis, inflammatory diseases, autoinflammatory diseases, and autoimmune diseases.

### Prophylactically Effective Amount

A “prophylactically effective amount” is an amount sufficient to prevent (e.g., significantly delay onset or recurrence of one or more symptoms or characteristics of, for example so that it/they is/are not detected at a time point at which they would be expected absent administration of the amount) a condition. A prophylactically effective amount of a composition means an amount of therapeutic agent(s), alone or in combination with other agents, that provides a prophylactic benefit in the prevention of the condition. The term “prophylactically effective amount” can encompass an amount that improves overall prophylaxis or enhances the prophylactic efficacy of another prophylactic agent. Those skilled in the art will appreciate that a prophylactically effective amount need not be contained in a single dosage form. Rather, administration of an effective amount may involve administration of a plurality of doses, potentially over time (e.g., according to a dosing regimen).

### Risk

As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0,1, 2, 3, 4, 5, 6, 7, 8, 9, 10, or more. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes.

### Salt

As used herein, the term “salt” refers to any and all salts and encompasses pharmaceutically acceptable salts.

### Sample

As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

### Small Molecule

The term “small molecule” or “small molecule therapeutic” refers to a molecule, whether naturally occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, a small molecule is a therapeutically active agent such as a drug (e.g., a molecule approved by the U.S. Food and Drug Administration as provided in the Code of Federal Regulations (C.F.R.)). A small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, a small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, the contents of each of which are incorporated herein by reference for purposes described herein; such listed drugs are typically considered acceptable for use in accordance with the present disclosure.

### Solvate

The term “solvate”, as used herein, has its art-understood meaning and refers to an aggregate of a compound (which may, for example, be a salt form of the compound) and one or more solvent atoms or molecules. In some embodiments, a solvate is a liquid. In some embodiments, a solvate is a solid form (e.g., a crystalline form). In some embodiments, a solid-form solvate is amenable to isolation. In some embodiments, association between solvent atom(s) and compound in a solvate is a non-covalent association. In some embodiments, such association is or comprises hydrogen bonding, van der Waals interactions, or combinations thereof. In some embodiments, a solvent whose atom(s) is/are included in a solvate may be or comprise one or more of water, methanol, ethanol, acetic acid, DMSO, THF, diethyl ether, and the like. Suitable solvates may be pharmaceutically acceptable solvates; in some particular embodiments, solvates are hydrates, ethanolates, or methanolates. In some embodiments, a solvate may be a stoichiometric solvate or a non-stoichiometric solvate.

### Subject

A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or a non-human animal, for example, a mammal (e.g., a primate (e.g., cynomolgus monkey, rhesus monkey); a domestic animal such as a cow, pig, horse, sheep, goat, cat, and/or dog; and/or a bird (e.g., a chicken, duck, goose, and/or turkey). In certain embodiments, the animal is a mammal (e.g., at any stage of development). In some embodiments, an animal (e.g., a non-human animal) may be a transgenic or genetically engineered animal. In some embodiments, a subject is a tumor resection subject, e.g., a subject who has recently undergone tumor resection. In some embodiments, a tumor resection subject is a subject who has undergone tumor resection in less than 72 hours (including, e.g., less than 48 hours, less than 24 hours, less than 12 hours, less than 6 hours, or lower) prior to receiving a composition described herein. In some embodiments, a tumor resection subject is a subject who has undergone tumor resection in less than 48 hours prior to receiving a composition described herein. In some embodiments, a tumor resection subject is a subject who has undergone tumor resection in less than 24 hours prior to receiving a composition described herein. In some embodiments, a tumor resection subject is a subject who has undergone tumor resection in less than 12 hours prior to receiving a composition described herein.

### Substantially

As used herein, the term “substantially” refers to the qualitative condition of exhibiting total or near-total extent or degree of a characteristic or property of interest. Those skilled in the art will understand that an agent of interest, if ever, achieves or avoids an absolute result, e.g., an agent of interest that indeed has zero effect on an immune response, e.g., inflammation. The term “substantially” is therefore used herein to capture the potential lack of absoluteness inherent in many biological and chemical effects.

### Sustained

As used interchangeably herein, the term “sustained” or “extended” typically refers to prolonging an effect and/or a process over a desirable period of time. For example, in the context of sustained immunomodulation (e.g., in the presence of a composition or preparation as described and/or utilized herein), such an immunomodulatory effect may be observed for a longer period of time after administration of a particular immunomodulatory payload in the context of a composition comprising a biomaterial preparation and otherwise as described herein, as compared to that which is observed with administration of the same payload absent such a biomaterial preparation. In the context of sustained release of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) from compositions and/or preparations described herein over a period of time, such release may occur on a timescale within a range of from about 30 minutes to several weeks or more. In some embodiments, the extent of sustained release or extended release can be characterized in vitro or in vivo. For example, in some embodiments, release kinetics can be tested in vitro by placing a preparation and/or composition described herein in an aqueous buffered solution (e.g., PBS at pH 7.4). In some embodiments, when a preparation and/or composition described herein is placed in an aqueous buffered solution (e.g., PBS at pH 7.4), less than 100% or lower (including, e.g., less than or equal to 90%, less than or equal to 80%, less than or equal to 70%, less than or equal to 50% or lower) of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) is released within 3 hours from a biomaterial. In some embodiments, release kinetics can be tested in vivo, for example, by implanting a composition at a target site (e.g., mammary fat pad) of an animal subject (e.g., a mouse subject). In some embodiments, when a composition is implanted at a target site (e.g., mammary fat pad) of an animal subject (e.g., a mouse subject), less than or equal to 70% or lower (including, e.g., less than or equal to 60%, less than or equal to 50%, less than 40%, less than 30% or lower) of one or more agents of interest (e.g., payloads incorporated in polymer combination preparations described herein and/or degradation or dissolution products and/or soluble components of polymer combination preparations described herein that modulate one or more aspects of an immune response, e.g., but not limited to innate immunity agonism) is released in vivo 8 hours after the implantation.

### Targeted Agent

The term “targeted agent”, when used in reference to an anticancer agent means one that blocks the growth and spread of cancer by interfering with specific molecules (“molecular targets”) that are involved in the growth, progression, and/or spread of cancer. Targeted agents are sometimes called “targeted cancer therapies,” “molecularly targeted drugs,” “molecularly targeted therapies,” or “precision medicines.” Targeted agents differ from traditional chemotherapy in that targeted agents typically act on specific molecular targets that are specifically associated with cancer, and/or with a particular tumor or tumor type, stage, etc., whereas many chemotherapeutic agents act on all rapidly dividing cells (e.g., whether or not the cells are cancerous). Targeted agents are deliberately chosen or designed to interact with their target, whereas many standard chemotherapies are identified because they kill cells.

### Tautomers

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

### Test Subject

As used herein, the term “test subject” refers to a subject to which technologies provided herein are applied for experimental investigation, e.g., to assess biomaterial degradation, and/or efficacy of compositions and/or preparations described herein in antitumor immunity. In some embodiments, a test subject may be a human subject or a population of human subjects. For example, in some embodiments, a human test subject may be a normal healthy subject. In some embodiments, a human test subject may be a tumor resection subject. In some embodiments, a test subject may be a mammalian non-human animal or a population of mammalian non-human animals. Non-limiting examples of such mammalian non-human animals include mice, rats, dogs, pigs, rabbits, etc., which in some embodiments may be normal healthy subjects, while in some embodiments may be tumor resection subjects. In some embodiments, mammalian non-human animals may be transgenic or genetically engineered animals.

### Therapeutic Agent

The term “therapeutic agent” refers to an agent having one or more properties that produce a desired, usually beneficial, physiological effect. For example, a therapeutic agent may treat, ameliorate, and/or prevent disease. Those skilled in the art, reading the present disclosure, will appreciate that the term “therapeutic agent”, as used herein, does not require a particular level or type of therapeutic activity, such as might be required for a regulatory agency to consider an agent to be “therapeutically active” for regulatory purposes. As will be understood by those skill in the art, reading the present disclosure, in some embodiments, certain polymer combination preparations described herein (in the absence of an immunomodulatory payload) may have one or more properties that contribute to and/or achieve a desired physiological effect, and therefore may be considered to be a “therapeutic agent” as that term is used here (whether or not such biomaterial would or would not be considered to be pharmaceutically active by any particular regulatory agency). In some embodiments, a therapeutic agent that may be utilized in preparations, compositions and/or methods described herein (e.g., involving polymer combination preparations described herein) does not comprise an immunomodulatory payload (e.g., as described herein). In some embodiments, a therapeutic agent that may be utilized in preparations, compositions and/or methods described herein (e.g., involving polymer combination preparations described herein) may be or comprise an immunomodulatory payload. In some embodiments, a therapeutic agent that may be utilized in preparations, compositions and/or methods described herein (e.g., involving polymer combination preparations described herein) may be or comprise a non-immunomodulatory payload, e.g., comprising a biologic, a small molecule, nucleic acid, polypeptide, or a combination thereof. In some embodiments, a therapeutic agent that may be utilized in preparations, compositions and/or methods described herein (e.g., involving polymer combination preparations described herein) may be or comprise a chemotherapeutic agent, which in some embodiments may be or comprise a cytotoxic agent.

### Therapeutically Effective Amount

A “therapeutically effective amount” is an amount sufficient to provide a therapeutic benefit in the treatment of a condition, which therapeutic benefit may be or comprise, for example, reduction in frequency and/or severity, and/or delay of onset of one or more features or symptoms associated with the condition. A therapeutically effective amount means an amount of therapeutic agent(s), alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. Those skilled in the art will appreciate that a therapeutically effective amount need not be contained in a single dosage form. Rather, administration of an effective amount may involve administration of a plurality of doses, potentially over time (e.g., according to a dosing regimen, and particularly according to a dosing regimen that has been established, when applied to a relevant population, to provide an appropriate effect with a desired degree of statistical confidence).

### Temperature-Responsive

As used herein, the term “temperature-responsive”, in the context of a temperature-responsive polymer or biomaterial (e.g., polymeric biomaterial), refers to a polymer or biomaterial (e.g., polymeric biomaterial) that exhibits an instantaneous or discontinuous change in one or more of its properties at a critical temperature (e.g., a critical gelation temperature). For example, in some embodiments, one or more of such properties is or comprise a polymer’s or biomaterial’s solubility in a particular solvent. By way of example only, in some embodiments, a temperature-responsive polymer or biomaterial (e.g., polymeric biomaterial) is characterized in that it is a homogenous polymer solution or colloid that is stable below a critical temperature (e.g., a critical gelation temperature) and instantaneously form a polymer network (e.g., a hydrogel) when the critical temperature (e.g., critical gelation temperature) has been reached or exceeded. In some embodiments, a temperature-responsive polymer or biomaterial (e.g., polymeric biomaterial) may be temperature-reversible, e.g., in some embodiments where a polymer solution may instantaneously form a polymer network at a temperature of or above a critical gelation temperature, and such a resulting polymer network may instantaneously revert to a homogenous polymer solution when the temperature is reduced to below the critical gelation temperature.

### Treat

The terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein, e.g., cancer or tumor. In some embodiments, treatment may be administered after one or more signs or symptoms have developed or have been observed. Treatment may also be continued after symptoms have resolved, for example, to delay or prevent recurrence and/or spread.

### Tumor

The terms “tumor” and “neoplasm” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor’s neoplastic cells, and these tumors are referred to as “pre-malignant neoplasms.” An example of a pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

### Tumor Removal

As used herein, the term “tumor removal” encompasses partial or complete removal of a tumor, which may be resulted from a cancer therapy, e.g., surgical resection. In some embodiments, tumor removal refers to physical removal of part or all of a tumor by surgery (i.e., “tumor resection”). In some embodiments, tumor removal may be resulted from a surgical tumor resection and an adjuvant therapy (e.g., chemotherapy, immunotherapy, and/or radiation therapy). In some embodiments, an adjuvant therapy may be administered after a surgical tumor resection, e.g., at least 24 hours or more after a surgical tumor resection.

### Tumor Resection Subject

As used herein, the term “tumor resection subject” refers to a subject who is undergoing or has recently undergone a tumor resection procedure. In some embodiments, a tumor resection subject is a subject who has at least 70% or more (including, at least 80%, at least 90%, at least 95%, at least 98%, at least 99%, or higher (including 100%) of gross tumor mass removed by surgical resection. Those of skill in the art will appreciate that, in some cases, there may be some residual cancer cells microscopically present at a visible resection margin even though gross examination by the naked eye shows that all of the gross tumor mass has been apparently removed. In some embodiments, a tumor resection subject may be determined to have a negative resection margin (i.e., no cancer cells seen microscopically at the resection margin, e.g., based on histological assessment of tissues surrounding the tumor resection site). In some embodiments, a tumor resection subject may be determined to have a positive resection margin (i.e., cancer cells are seen microscopically at the resection margin, e.g., based on histological assessment of tissues surrounding the tumor resection site). In some embodiments, a tumor resection subject may have micrometastases and/or dormant disseminated cancer cells that can be driven to progress/proliferate by the physiologic response to surgery. In some embodiments, a tumor resection subject receives a composition (e.g., as described and/or utilized herein) immediately after the tumor resection procedure is performed (e.g., intraoperative administration). In some embodiments, a tumor resection subject receives a composition (e.g., as described and/or utilized herein) postoperatively within 24 hours or less, including, e.g., within 18 hours, within 12 hours, within 6 hours, within 3 hours, within 2 hours, within 1 hour, within 30 mins, or less.

### Tumor site

The term “tumor site” may, in some embodiments, be a site in which at least a portion of a tumor is present or was present prior to resection. In some embodiments, a tumor site may still have the entirety of the tumor present. While in some embodiments, a tumor site may have part or all of the tumor removed, e.g., through tumor resection.

### Variant

As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant biomaterial (e.g., a variant polymer or a polymeric biomaterial comprising a variant polymer) may differ from a reference biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can retain the desired property(ies) and/or function(s) (e.g., immunomodulation and/or temperature-responsiveness) of the reference biomaterial. For example, a variant of an immunomodulatory biomaterial may differ from a reference immunomodulatory biomaterial (e.g., a reference polymer or polymeric biomaterial) as a result of one or more structural modifications (e.g., but not limited to, additions, deletions, and/or modifications of chemical moieties, and/or grafting) provided that the variant biomaterial (e.g., variant polymer or polymeric biomaterial comprising such a variant polymer) can act on an immune system (e.g., by stimulating innate immunity), e.g., when used in a method described herein. In some embodiments, a variant immunomodulatory biomaterial (e.g., a variant polymer or a polymeric biomaterial comprising a variant polymer) is characterized in that, when assessed at 24 hours after administration of such a variant immunomodulatory biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 60% or more (e.g., including, e.g., at least 70%, at least 80%, at least 90%, at least 95%, at least 98%, or up to 100%) of that observed when a reference biomaterial (e.g., a reference polymer or polymeric biomaterial) is administered at the target site. In some embodiments, a variant immunomodulatory biomaterial (e.g., a variant polymer or a polymeric biomaterial comprising a variant polymer) is characterized in that, when assessed at 24 hours after administration of such a variant biomaterial (e.g., a variant polymer or a polymeric biomaterial comprising a variant polymer) to a target site in a subject, an amount of one or more proinflammatory cytokines (e.g., but not limited to CXCL10, IFN-α, IFN-β, IL-1β, IL-6, IL-18, and/or TNF-α) observed at the target site and/or body circulation of the subject is at least 1.1-fold or more (e.g., including, e.g., at least 1.5-fold, at least 2-fold, at least 3-fold, at least 4-fold, at least 5-fold, or more) of that observed when a reference biomaterial (e.g., a reference polymeric biomaterial) is administered at the target site. In some embodiments, a variant biomaterial (e.g., a variant polymeric biomaterial) exhibits at least one physical characteristic that is different from that of a reference biomaterial (e.g., a reference polymeric biomaterial). For example, in some embodiments, a variant biomaterial (e.g., a variant polymeric biomaterial) can exhibit increased water solubility (e.g., at a physiological pH) as compared to that of a reference biomaterial (e.g., a reference polymeric biomaterial). In some embodiments, a variant has 10, 9, 8, 7, 6, 5, 4, 3, 2, or 1 structural modifications as compared with a reference. In some embodiments, a variant has a small number (e.g., fewer than 5, 4, 3, 2, or 1) number of structural modifications (e.g., alkylation, acylation, quaternization, hydroxyalkylation, carboxyalkylation, thiolation, phosphorylation, glycosylation, etc.). In some embodiments, a variant has not more than 5, 4, 3, 2, or 1 additions or deletions of chemical moieties, and in some embodiments has no additions or deletions, as compared with a reference. In some embodiments, a variant is an entity that can be generated from a reference by chemical manipulation. In some embodiments, a variant is an entity that can be generated through performance of a synthetic process substantially similar to (e.g., sharing a plurality of steps with) one that generates a reference.

### DETAILED DESCRIPTION OF CERTAIN EMBODIMENTS

The present disclosure, among other things, provides technologies, including, e.g., particular biomaterial preparations, that may be particularly useful and/or may provide particular beneficial effects, e.g., as described herein.

Among other things, the present disclosure appreciates that certain crosslinking technologies may produce toxic by-products and/or may adversely affect stability and/or efficacy of agent(s) (e.g., therapeutic agents) that may be combined with biopolymer materials before or during crosslinking. In some embodiments, the present disclosure, among other things, provides technologies that address such a problem with certain prior technologies including, for example, with certain crosslinked biopolymer materials.

Alternatively or additionally, the present disclosure appreciates that technologies involving pre-forming (e.g., by chemical cross-linking) a biopolymer material prior to introducing into a subject generate a material with a defined size and/or structure, which may restrict options for administration. The present disclosure provides technologies, including particular biomaterial preparations, that permit administration by a variety of routes and/or approaches, including by methods, such as injection and/or laparoscopic administration, that may be less invasive than implantation. In some such embodiments, preparations with improved administration characteristics may be administered in a liquid state; in some embodiments they may be administered in a pre-formed gel state characterized by flexible space-filling properties. In some such embodiments, provided preparations are comprised of a relevant material in particulate form (e.g., so that the preparations comprise a plurality of particles, e.g., characterized by a size distribution and/or other parameters as described herein).

Among other things, in some embodiments, the present disclosure provides temperature-responsive biomaterial preparations that, for example are able to transition from an injectable state to a polymer network state with material properties that provide beneficial effects, e.g., as described herein, without introduction of a cytotoxic crosslinking agent, e.g., UV radiation and/or chemical crosslinkers (e.g., small-molecule crosslinkers). Some such embodiments, thus provide valuable technologies for in situ formation of gelled materials, which technologies have various benefits relative to alternative technologies, and provide a solution to certain problems with such alternative technologies as identified herein. For example, the present disclosure identifies the source of a problem with various alternative technologies for in situ gelation, as many such technologies require treatments (e.g., exposure to UV radiation and/or to a chemical crosslinker, e.g., a small-molecule crosslinker), that may have toxic or otherwise damaging effects for the recipient and/or for an agent that may be included in or with the material.

In some embodiments, the present disclosure appreciates, among other things, that certain conventional preparations that are or comprise a poloxamer and that are used to form a hydrogel typically utilize such that are or comprise a poloxamer (e.g., Poloxamer 407 (P407)) at a minimum concentration of 16-20% (w/w). The present disclosure identifies the source of a problem with such conventional preparations, including that they may have certain disadvantages for administration to subjects, including, e.g., high solution viscosity that makes it less ideal for injection, and/or tissue irritation due to high concentrations of poloxamers. Moreover, the present disclosure demonstrates that it is possible to develop useful preparations with materially lower concentration(s) of such poloxamers.

For example, in some embodiments, the present disclosure provides an insight that certain poloxamers, e.g., Poloxamer 407 (P407), which have been typically used at a minimum concentration of 16-20% (w/w) to form a hydrogel, can form a useful temperature-responsive biomaterial at concentrations lower than 16% (w/w), including, e.g., lower than 14% (w/w), lower than 12% (w/w), lower than 11% (w/w), lower than 10.5% (w/w), lower than 10% (w/w), lower than 9% (w/w), lower than 8% (w/w), lower than 7% (w/w), or lower than 6% (w/w), lower than 5%(w/w) when combined with one or more biocompatible polymers. In some embodiments, such biocompatible polymers may be or comprise a polymer that is not temperature- responsive, e.g., in some embodiments which may be or comprise hyaluronic acid and/or chitosan or modified chitosan.

Alternatively or additionally, in some embodiments, the present disclosure provides an insight that softer hydrogels may provide greater therapeutic benefits than high-concentration P407 hydrogels (e.g., at a concentration of 16-20% (w/w)) and/or chemically-crosslinked hydrogels. For example, in some embodiments, the present disclosure demonstrates that certain polymer combination preparations described herein with a lower storage modulus that are incorporated with an immunomodulatory payload can provide greater survival benefits in tumor resection animals, as compared to that is observed in tumor resection animals receiving, e.g., a chemically crosslinked hyaluronic acid hydrogel incorporated with the same immunomodulatory payload.

In some embodiments, provided temperature-responsive biomaterial preparations (e.g., ones described herein) may demonstrate one or more immunomodulatory attributes, even in the absence of an immunomodulatory payload. In some embodiments, a biomaterial preparation comprising a poloxamer at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4% (w/w), or lower) and at least one additional polymer that is not poloxamer may be immunomodulatory itself in the absence of an immunomodulatory payload. For example, in some embodiments, such a biomaterial preparation comprising a poloxamer at a concentration of 12.5% (w/w) or below (e.g., 11% (w/w), 10.5% (w/w), 10% (w/w), 9% (w/w), 8% (w/w), 7% (w/w), 6% (w/w), 5% (w/w), 4% (w/w), or lower) and at least one carbohydrate polymer (e.g., hyaluronic acid or chitosan) may promote innate immunity upon administration to a target site in subject in need thereof (e.g., tumor resection subjects).

### I. Compositions or Preparations Comprising Provided Polymer Combination Preparations

In some embodiments, the present disclosure, among other things, provides compositions and/or preparations comprising polymer combination preparations (e.g., ones described herein) that are temperature-responsive, which thus permit in situ gelation at a target site in the absence of crosslinking treatments (e.g., introduction of UV radiation and/or chemical crosslinkers) that may have toxic or otherwise damaging effects for the recipient and/or for a payload that may be included in or with a biomaterial.

In some embodiments, the present disclosure provides compositions and/or compositions comprising certain polymer combination preparations that are useful to provide a sustained release of payloads incorporated in polymer combination preparations. For example, in some embodiments, certain compositions and/or preparations described herein can be remarkably useful when such compositions incorporating one or more immunomodulatory payloads (e.g., ones as described herein and/or as described in WO 2018/045058, the contents of which are incorporated herein by reference for the purposes described herein) are administered to subjects who have undergone or are undergoing a tumor resection. By way of example only, in some embodiments, a composition or preparation of the present disclosure may comprise at least one innate immunity modulatory payload. In some embodiments, a composition or preparation of the present disclosure may comprise at least one innate immunity modulatory payload and at least one adaptive immunity modulatory payload. In some embodiments, a composition or preparation of the present disclosure may comprise at least one innate immunity modulatory payload, at least one adaptive immunity modulatory payload, and at least one immunomodulatory cytokine. In some embodiments, a composition or preparation of the present disclosure may comprise at least one inhibitor of a proinflammatory immune response.

In some embodiments, the present disclosure provide compositions and/or compositions comprising certain polymer combination preparations that by themselves are sufficient to provide an immunomodulatory response (e.g., to provide sufficient innate immunity agonism) to achieve beneficial effects even absent a separate immunomodulatory payload. In some embodiments, not only is a polymer combination preparation described and/or utilized herein substantially free of an immunomodulatory payload (e.g., an innate immunity modulatory payload), but also such a composition or preparation of the present disclosure may not necessarily require inclusion of at least one or more (e.g., at least two or more, at least three or more) other types of immunomodulatory payloads, including, e.g., adaptive immunity modulatory payloads, immunomodulatory cytokines, immunomodulatory chemotherapeutics, immunomodulatory therapeutic agents, and/or combinations thereof. By way of example only, in some embodiments, a composition or preparation of the present disclosure is substantially free of an innate immunity modulatory payload and an adaptive immunity modulatory payload. In some embodiments, a composition or preparation of the present disclosure is substantially free of an innate immunity modulatory payload, an adaptive immunity modulatory payload, and an immunomodulatory cytokine. In some embodiments, a composition or preparation of the present disclosure is substantially free of an inhibitor of a proinflammatory response. In some embodiments, a composition or preparation of the present disclosure comprises a provided polymer combination preparation in the absence of an immunomodulatory payload.

In some embodiments, a polymer combination preparation can comprise a biomaterial preparation and a payload agent which, in many embodiments, is an immune system modulator as described herein (e.g., an immunomodulatory payload). Alternatively, in some embodiments, a utilized immunomodulatory composition comprising a polymer combination preparation may be substantially free of a known immunomodulatory payload.

In some embodiments, a polymer combination preparation described herein is characterized in that it forms a polymer network; without wishing to be bound by any particular theory, it is noted that, in some embodiments, such a network may act as a scaffold or depot for a payload (e.g., for an immunomodulatory payload) within a polymer combination preparation.

In some embodiments, a polymer combination preparation comprising a biomaterial preparation and a payload agent (e.g., in some embodiments, an immunomodulatory payload) may perform as an extended release formulation, for example, in that the payload is released from the composition more slowly (i.e. over a longer period of time) than is observed for an otherwise comparable composition lacking the polymer combination preparation (e.g., lacking one or all polymer components thereof).

In some embodiments, a polymer combination preparation for use as described herein comprises one or more polymers (e.g., ones described herein). In certain embodiments, a polymer combination preparation may comprise one or more positively charged polymers. In some embodiments, a polymer combination preparation for use as described herein may comprise one or more negatively charged polymers. In some embodiments, a polymer combination preparation for use as described herein may comprise one or more neutral polymers.

### Provided Polymer Combination Preparations

In some embodiments, the present disclosure, among other things, provides polymer combination preparations comprising at least first and second polymer components, wherein the first polymer component is or comprises a poloxamer (e.g., ones described herein) and the second polymer component is not a poloxamer, wherein the first polymer component is present in the polymer combination preparation at a concentration of 12.5% (w/w) or below. In some embodiments, such polymer combination preparation is characterized in that it transitions from a precursor state to a polymer network state in response to a gelation trigger, which is or comprises one or more of the following: (a) temperature at or above critical gelation temperature (CGT) for the polymer combination preparation, (b) critical gelation weight ratio of the first polymer component to the second polymer component, (c) total polymer content, (d) molecular weights of the first and/or second polymer components, or (e) combinations thereof. A polymer network state of a provided polymer combination preparation has a viscosity materially above that of the precursor state and comprises crosslinks not present in the precursor state. In some embodiments, a precursor state of a provided polymer combination preparation is a liquid state. In some embodiments, a precursor state of a provided polymer combination preparation is an injectable state. In some embodiments, a polymer network state of a provided polymer combination preparation is a more viscous liquid state. In some embodiments, a polymer network state of a provided polymer combination preparation is a hydrogel.

In some embodiments, a provided polymer combination preparation is temperature-responsive, so that, e.g., its gelation (e.g., its transition from a liquid state to a gelled state) can occur upon exposure to a particular temperature. In many such embodiments, exposure to body temperature (e.g., by application to a site) is sufficient to trigger such gelation; in some embodiments, additional warmth may be applied. By way of example only, in some embodiments, a temperature-responsive polymer combination preparation as described herein is characterized in that it transitions from a precursor state (e.g., a liquid state or an injectable state) to a polymer network state that has a viscosity and/or storage modulus materially above that of the precursor state (e.g., a more viscous state or a hydrogel) when such a polymer combination preparation is exposed to a gelation trigger, which is or comprises a temperature at or above critical gelation temperature (CGT) for the polymer combination preparation. In some embodiments, a CGT for a provided polymer combination preparation is at least 10° C. or higher, including e.g. at least 10° C., at least 11° C., at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., 33° C., at least 34° C., at least 35° C., at least 36° C., at least 37° C., at least 38° C., at least 39° C., at least 40° C., or higher. In some embodiments, a CGT for a provided polymer combination preparation is about 10° C. to about 15° C. In some embodiments, a CGT for a provided polymer combination preparation is about 12° C. to about 17° C. In some embodiments, a CGT for a provided polymer combination preparation is about 14° C. to about 19° C. In some embodiments, a CGT for a provided polymer combination preparation is about 16° C. to about 21° C. In some embodiments, a CGT for a provided polymer combination preparation is about 18° C. to about 23° C. In some embodiments, a CGT for a provided polymer combination preparation is about 20° C. to about 25° C. In some embodiments, a CGT for a provided polymer combination preparation is about 22° C. to about 27° C. In some embodiments, a CGT for a provided polymer combination preparation is about 24° C. to about 29° C. In some embodiments, a CGT for a provided polymer combination preparation is about 26° C. to about 31° C. In some embodiments, a CGT for a provided polymer combination preparation is about 28° C. to about 33° C. In some embodiments, a CGT for a provided polymer combination preparation is about 30° C. to about 35° C. In some embodiments, a CGT for a provided polymer combination preparation is about 32° C. to about 37° C. In some embodiments, a CGT for a provided polymer combination preparation is about 34° C. to about 39° C. In some embodiments, a CGT for a provided polymer combination preparation is about 35° C. to about 39° C. In some embodiments, a CGT for a provided polymer combination preparation is at or near physiological temperature of a subject (e.g., a human subject) receiving such a polymer combination preparation.

In some embodiments, a provided polymer combination preparation is temperature-reversible. For example, in some embodiments, a provided polymer combination preparation is characterized in that it transitions from a precursor state (e.g., a liquid state or an injectable state) to a polymer network state that has a viscosity and/or storage modulus materially above that of the precursor state (e.g., a more viscous state or a hydrogel) when such a polymer combination preparation is exposed to a temperature at or above critical gelation temperature (CGT) for the polymer combination preparation; and it may revert from the polymer network state to a state that has a viscosity and/or storage modulus materially lower than that of the polymer network state (e.g., a liquid state or original state of a provided polymer combination preparation).

In some embodiments, a polymer combination preparation described herein does not comprise a chemical crosslinker. Those of skill in the art will appreciate that, in some embodiments, a chemical crosslinker is characterized in that it facilitates formation of covalent crosslinks between polymer chains. In some embodiments, a chemical crosslinker is or comprises a small-molecule crosslinker, which can be derived from a natural source or synthesized. Non-limiting examples of small-molecule crosslinkers include genipin, dialdehyde, glutaraldehyde, glyoxal, diisocyanate, glutaric acid, succinic acid, adipic acid, acrylic acid, diacrylate, etc.). In some embodiments, a chemical crosslinker may involve crosslinking using thiols (e.g., EXTRACEL®, HYSTEM®), methacrylates, hexadecylamides (e.g., HYMOVIS®), and/or tyramines (e.g., CORGEL®). In some embodiments, a chemical crosslinker may involve crosslinking using formaldehyde (e.g., HYLAN-A®), divinylsulfone (DVS) (e.g., HYLAN-B®), 1,4-butanediol diglycidyl ether (BDDE) (e.g., RESTYLANE®), glutaraldehyde, and/or genipin (see, e.g., Khunmanee et al. “Crosslinking method of hyaluronic-based hydrogel for biomedical applications” J Tissue Eng. 8: 1-16 (2017)). Accordingly, in some embodiments, crosslinks that form during the transition from a precursor state to a polymer network state do comprise covalent crosslinks.

In some embodiments, a first polymer component (e.g., poloxamer described herein) and a second polymer component (e.g., ones described herein) are present in a polymer combination preparation at a critical gelation weight ratio of 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 12:1; 13:1, 14:1, 15:1, 16:1, 17:1, 18:1, 19:1, or 20:1. In some embodiments, a first polymer component (e.g., poloxamer described herein) and a second polymer component (e.g., ones described herein) are present in a polymer combination preparation at a critical gelation weight ratio of 1:1 to 20:1 or 1:1 to 18:1,or 1:1 to 14:1, or 1.5:1 to 14:1, or 2:1 to 13:1, or 1:1 to 10:1, or 2:1 to 20:1 or 2:1 to 18:1, or 2:1 to 10:1. In some embodiments, a first polymer component (e.g., poloxamer described herein) and a second polymer component (e.g., ones described herein) are present in a polymer combination preparation at a critical gelation weight ratio of 1:1 to 10:1. In some embodiments, a first polymer component (e.g., poloxamer described herein) and a second polymer component (e.g., ones described herein) are present in a polymer combination preparation at a critical gelation weight ratio of 2:1 to 10:1. In some embodiments, a first polymer component (e.g., poloxamer described herein) and a second polymer component (e.g., ones described herein) are present in a polymer combination preparation at a critical gelation weight ratio such that the second polymer component may be in a greater amount by weight than that of the first polymer component. For example, in some embodiments, a first polymer component (e.g., poloxamer described herein) and a second polymer component (e.g., ones described herein) are present in a polymer combination preparation at a critical gelation weight ratio of 1:1.1, 1:1.2, 1:1.3, 1:1.4, 1:1.5, 1:1.6, 1:1.7, 1:1.8, 1:1.9, 1:2, etc. In some such embodiments, poloxamer concentration may be less than 7% (w/w) or lower, e.g., 6% (w/w), 5% (w/w), 4% (w/w), or lower.

In some embodiments, a polymer combination preparation provided herein comprising at least first and second polymer components (e.g., ones described herein) may comprise at least one additional polymer components, including, e.g., at least one, at least two, at least three, at least four, at least five, at least six, or more additional polymer components, which in some embodiments may be or comprise a biocompatible and/or biodegradable polymer component (e.g., as described herein).

In some embodiments, a provided polymer combination preparation comprises total polymer content of at least 5% (w/w) or higher, including, e.g., at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), at least 20% (w/w), or higher. In some embodiments, a provided polymer combination preparation comprises total polymer content of 5% (w/w) to 20% (w/w), or 6% (w/w) to 18% (w/w), or 8% (w/w) to 15% (w/w), or 9% (w/w) to 12% (w/w). In some embodiments, a polymer combination preparation described herein comprises a total polymer content of 6% (w/w) to 20% (w/w), or 8% (w/w) to 20% (w/w), or 10% (w/w) to 15% (w/w).

In some embodiments, a first polymer component, which is or comprises a poloxamer, is present in a provided polymer combination preparation at a concentration of no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w)), no more than 7% (w/w), no more than 6% (w/w), no more than 5% (w/w), or no more than 4% (w/w). In some embodiments, a first polymer component, which is or comprises a poloxamer, is present in a provided polymer combination preparation at a concentration of 5% (w/w) to 12.5% (w/w), or 8% (w/w) to 12.5% (w/w), or 5% (w/w) to 11% (w/w), or 5% (w/w) to 10% (w/w), or 6% (w/w) to 10% (w/w), or 8% (w/w) to 10% (w/w). In some embodiments, a first polymer component, which is or comprises a poloxamer, is present in a provided polymer combination preparation at a concentration of 4% (w/w) to 12.5% (w/w), or 4% (w/w) to 11% (w/w), or 4% (w/w) to 10.5% (w/w), or 4% (w/w) to 10% (w/w). In some embodiments, a first polymer component, which is or comprises a poloxamer, is present in a provided polymer combination preparation at a concentration of 5% (w/w) to 12.5% (w/w), or 5% (w/w) to 11% (w/w), or 5% (w/w) to 10.5% (w/w), or 5% (w/w) to 10% (w/w). In some embodiments, a first polymer component, which is or comprises a poloxamer, is present in a provided polymer combination preparation at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a second polymer component may be present in a provided polymer combination preparation at a concentration of no more than 15% (w/w). In some embodiments, a second polymer component may be present in a provided polymer combination preparation at a concentration of no more than 10% (w/w), including, e.g., at a concentration of 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or lower. In some embodiments, a second polymer component may be present in a provided polymer combination preparation at a concentration of at least 0.1% (w/w), including, e.g., at least 0.2% (w/w), at least 0.3% (w/w), at least 0.4% (w/w), at least 0.5% (w/w), at least 0.6% (w/w), at least 0.7% (w/w), at least 0.8% (w/w), at least 0.9% (w/w), at least 1% (w/w), at least 1.5% (w/w), at least 2% (w/w), at least 2.5% (w/w), at least 3% (w/w), at least 3.5% (w/w), at least 4% (w/w), at least 4.5% (w/w), at least 5% (w/w), at least 6% (w/w), at least 7% (w/w), at least 8% (w/w), at least 9% (w/w), at least 10% (w/w), or higher. In some embodiments, a second polymer component in a provided polymer combination preparation may be present at a concentration of 0.1% (w/w) to 10% (w/w), or 0.1% (w/w) to 8% (w/w), or 0.1% (w/w) to 5% (w/w), or 1% (w/w) to 5% (w/w). In some embodiments, a second polymer component in a provided polymer combination preparation may be present at a concentration of 0.5% (w/w) to 10% (w/w), or 0.5% (w/w) to 5% (w/w), or 1% (w/w) to 10% (w/w), or 1% (w/w) to 5% (w/w), or 2% to 10% (w/w).

### A. First Polymer Component Comprising One or More Exemplary Poloxamers And Variants Thereof

In some embodiments, a provided polymer combination preparation comprises a poloxamer or a variant thereof. Poloxamer is typically a block copolymer comprising a hydrophobic chain of polyoxypropylene (e.g., polypropylene glycol, PPG, and/or poly(propylene oxide), PPO) flanked by two hydrophilic chains of polyoxyethylene (e.g., polyethylene glycol, PEG, and/or poly(ethylene oxide), PEO). Poloxamers are known by the trade names Synperonic, Pluronic, and/or Kolliphor. Generally, poloxamers are non-ionic surfactants, which in some embodiments may have a good solubilizing capacity, low toxicity, and/or high compatibility with cells, body fluids, and a wide range of chemicals.

In some embodiments, a poloxamer for use in accordance with the present disclosure may be a poloxamer known in the art. For example, as will be understood by a skilled person in the art, poloxamers are commonly named with the letter P (for poloxamer) followed by three digits: the first two digits multiplied by 100 give the approximate molecular mass of the polyoxypropylene chain, and the last digit multiplied by 10 gives the percentage polyoxyethylene content. By way of example only, P407 refers to a poloxamer with a polyoxypropylene molecular mass of 4,000 g/mol and a 70% polyoxyethylene content). A skilled person in the art will also understand that for the Pluronic and Synperonic tradenames, coding of such poloxamers starts with a letter to define its physical form at room temperature (e.g., L = liquid, P = paste, F = flake (solid)) followed by two or three digits, wherein the first digit (two digits in a three-digit number) in the numerical designation, multiplied by 300, indicates the approximate molecular weight of the polyoxypropylene chain; and the last digit, multiplied by 10, gives the percentage polyoxyethylene content. By way of example only, L61 refers to a liquid preparation of poloxamer with a polyoxypropylene molecular mass of 1,800 g/mol and a 10% polyoxyethylene content. In addition, as will be apparent to a skilled artisan, poloxamer 181 (P181) is equivalent to Pluronic L61 and Synperonic PE/L61.

In some embodiments, a poloxamer that may be included in a polymer combination preparation described herein may be or comprise Poloxamer 124 (e.g., Pluronic L44 NF), Poloxamer 188 (e.g., Pluronic F68NF), Poloxamer 181 (e.g., Pluronic L61), Poloxamer 182 (e.g., Pluronic L62), Poloxamer 184 (e.g., Pluronic L64), Poloxamer 237 (e.g., Pluronic F87 NF), Poloxamer 338 (e.g., Pluronic F108 NF), Poloxamer 331 (e.g., Pluronic L101), Poloxamer 407 (e.g., Pluronic F127 NF), or combinations thereof. In some embodiments, a provided polymer combination preparation can comprise at least two or more different poloxamers. Additional poloxamers as described in Table 1 of Russo and Villa “Poloxamer Hydrogels for Biomedical Applications” Pharmaceutics (2019) 11(12):671, the contents of which are incorporated herein by reference for the purposes described herein, may be also useful for polymer combination preparations described herein.

In some embodiments, a poloxamer that may be included in a polymer combination preparation described herein may be or comprise Poloxamer 407 (P407). In some embodiments, P407 is a triblock poloxamer copolymer having a hydrophobic PPO block flanked by two hydrophilic PEO blocks. The approximate length of the two PEO blocks is typically 101 repeat units, while the approximate length of the PPO block is 56 repeat units. In some embodiments, P407 has an average molecular weight of approximately 12,600 Da of which approximately 70% corresponds to PEO. In some embodiments, P407 can readily self-assemble to form micelles dependent upon concentration and ambient temperature. Without wishing to be bound by a particular theory, dehydration of hydrophobic PPO blocks combined with hydration of PEO blocks may lead to formation of spherical micelles, and subsequent packing of the micellar structure results in a 3D cubic lattice that constitutes the main structure of poloxamer hydrogels. They are also biodegradable, non-toxic, and stable, and are therefore suitable for use as controlled release of therapeutic agents. As appreciated by one of ordinary skill in the art, P407 concentrations in hydrogel formulations based on binary poloxamer/water mixtures are typically in the range from 16-20w/v%, with a value of approximately 18% w/v most frequently used. See, e.g., Pereia et al. “Formulation and Characterization of Poloxamer 407®: Thermoreversible Gel Containing Polymeric Microparticles and Hyaluronic Acid” Quim. Nova, Vol. 36, No. 8, 1121-1125 (2013), the contents of which are incorporated herein by reference in their entirety.

Various crosslinking approaches, e.g., chemical crosslinking and enzyme-mediated crosslinking approaches, were used to crosslink P407 alone or in combination with another polymer at a P407 concentration lower than a typical range of 16-20w/v%. See, e.g., Ryu et al. “Catechol-functionalized chitosan/pluronic hydrogels for tissue adhesives and hemostatic materials” Biomacromolecules (2011) 12(7): 2653-2659; Lee et al. “Thermo-sensitive, injectable, and tissue adhesive sol-gel transition hyaluronic acid/pluronic composite hydrogels prepared from bio-inspired catechol-thiol reaction” Soft Matter (2010) 6: 977-983; and Chung et al. “Thermo-sensitive biodegradable hydrogels based on stereocomplexed pluronic multi-block copolymers for controlled protein delivery” J Control Release (2008) 127: 22-30; and Lee et al. “Enzyme-mediated cross-linking of pluronic copolymer micelles for injectable and in situ forming hydrogels” Acta Biomater (2011) 7: 1468-76, the contents of each of which are incorporated by reference in their entirety. However, in some embodiments, such crosslinking approaches require use of a chemical crosslinker or an enzyme, and/or a modified P407, which may not be desirable for in vivo administration. In some embodiments, the present disclosure, among other things, provides an insight that certain polymer combination preparations (e.g., ones described herein) may be particularly useful to form temperature-responsive hydrogels in the absence of chemical crosslinks or enzyme-mediated crosslinks, while the concentration of P407 is no more than 12.5% (w/w) (including, e.g., no more than 12% (w/w), no more than 11.5% (w/w), no more than 11% (w/w), no more than 10.5% (w/w), no more than 10% (w/w), no more than 9.5% (w/w), no more than 9% (w/w), no more than 8% (w/w)) in the polymer combination preparation. In some embodiments, the concentration of P407 is present in a provided polymer combination at a concentration of 5% (w/w) to 12.5% (w/w), or 5% (w/w) to 11% (w/w), or 8% (w/w) to 12.5% (w/w), or 5% (w/w) to 10% (w/w), or 8% (w/w) to 10% (w/w) or 6% (w/w) to 10% (w/w). In some embodiments, the concentration of P407 is present in a provided polymer combination at a concentration of 4% (w/w) to 12.5% (w/w), or 4% (w/w) to 11% (w/w), or 4% (w/w) to 10.5% (w/w), or 4% (w/w) to 10% (w/w). In some embodiments, the concentration of P407 is present in a provided polymer combination at a concentration of 5% (w/w) to 12.5% (w/w), or 5% (w/w) to 11% (w/w), or 5% (w/w) to 10.5% (w/w), or 5% (w/w) to 10% (w/w). In some embodiments, the concentration of P407 is present in a provided polymer combination at a concentration of 6% (w/w) to 12.5% (w/w), or 6% (w/w) to 11% (w/w), or 6% (w/w) to 10.5% (w/w), or 6% (w/w) to 10% (w/w).

In some embodiments, a P407 that may be included in a polymer combination preparation described herein may be or comprise compendial poloxamer 407. In some embodiments, such compendial poloxamer 407 included in a provided polymer combination preparation has not undergone additional purification steps. In some embodiments, such compendial poloxamer 407 included in a provided polymer combination preparation has not been modified, e.g., in some embodiments genetically modified. In some embodiments, a P407 that may be useful in a polymer combination preparation described herein may have a sol-to-gel transition temperature (T_{sol-gel}) in PBS of at least 18° C. or higher, including, e.g., 18.5° C., 19° C., 19.5° C., 20° C., 20.5° C., 21° C., 21.5° C., 22° C., 22.5° C., 23° C., or 23.5°. In some embodiments, a P407 that may be useful in a polymer combination preparation described herein may have an average molecule weight of no more than 12 kDa, e.g., no more than 11.5 kDa, no more than 11 kDa, no more than 10.5 kDa, or lower. As understood by one of ordinary skill in the art, the Tₛₒₗ₋_{gel} and/or average molecule weight of P407 in PBS may be varied by purification. For example, in some embodiments, the T_{sol-gel} and/or average molecule weight of P407 in PBS may increase when low molecular weight copolymer molecules and/or impurities are removed from compendial P407. Alternatively, the T_{sol-gel} and/or average molecule weight of P407 in PBS may decrease when high molecular weight copolymer molecules and/or impurities are removed from compendial P407. See, e.g., Fakhari et al. “Thermogelling properties of purified poloxamer 407” Heliyon (2017) 3(8):e00390, the contents of which are incorporated herein by reference in their entirety.

In some embodiments, a P407 to be included in a polymer combination preparation described herein may be a non-conjugated or non-modified P407 (e.g., a P407 that is not covalently conjugated to a moiety, such as, e.g., a polymer or an amino acid). Examples of conjugated P407 include, but are not limited to grafting P407 onto a carbohydrate polymer, e.g., chitosan, or a thiolated P407). See, e.g., Park et al. “Thermosensitive chitosan-Pluronic hydrogel as an injectable cell delivery carrier for cartilage regeneration” Acta Biomaterialia (2009) 5(6): 1956-1965; and Ryu et al. “Catechol-functionalized chitosan/pluronic hydrogels for tissue adhesives and hemostatic materials” Biomacromolecules (2011) 12(7): 2653-2659, the contents of each of which are incorporated herein by reference in their entirety.

In some embodiments, a poloxamer that may be included in a polymer combination preparation described herein may be or comprise Poloxamer 338.

In some embodiments, a poloxamer that may be included in a polymer combination preparation described herein may be or comprise Poloxamer 331.

In some embodiments, a poloxamer that may be included in a polymer combination preparation described herein may be or comprise Poloxamer 237.

In some embodiments, a poloxamer that may be included in a polymer combination preparation described herein may be or comprise Poloxamer 188.

In some embodiments, a poloxamer that may be included in a polymer combination preparation described herein may be or comprise Poloxamer 184.

In some embodiments, a poloxamer that may be included in a polymer combination preparation described herein may be or comprise Poloxamer 182.

In some embodiments, a poloxamer that may be included in a polymer combination preparation described herein may be or comprise Poloxamer 181.

In some embodiments, a poloxamer that may be included in a polymer combination preparation described herein may be or comprise Poloxamer 124.

In some embodiments, a poloxamer that may be included in a polymer combination preparation described herein may have a polyoxyethylene content of at least 30% by weight, including, e.g., at least 40%, at least 50%, at least 60%, at least 70%, at least 80%, at least 90%, or higher by weight. In some embodiments, a poloxamer may have a polyoxyethylene content of 50-90% by weight. In some embodiments, a poloxamer has a polyoxyethylene content of 60-90%. In some embodiments, a poloxamer has a polyoxyethylene content of 70-90%. In some embodiments, a poloxamer has a polyoxyethylene content of about 70%. In some embodiments, a poloxamer has a polyoxyethylene content of about 80%.

In some embodiments, a poloxamer that may be included in a polymer combination preparation described herein may have an average molecular weight of at least 1,500 g/mol or higher, including, e.g., at least 2,000 g/mol, at least 2,500 g/ml, at least 3,000 g/mol, at least 4,000 g/mol, at least 5,000 g/mol, at least 6,000 g/mol, at least 7,000 g/mol, at least 8,000 g/mol, at least 9,000 g/mol, at least 10,000 g/mol, at least 11,000 g/mol, at least 12,000 g/mol, at least 13,000 g/mol, at least 14,000 g/mol, at least 15,000 g/mol, at least 16,000 g/mol, at least 17,000 g/mol, at least 18,000 g/mol, at least 19,000 g/mol, at least 20,000 g/mol, or higher. In some embodiments, a poloxamer may have an average molecular weight between about 1,500 and 20,000 g/mol. In some embodiments, a poloxamer may have an average molecular weight between about 4,000 and 12,000 g/mol. In some embodiments, a poloxamer may have an average molecular weight between about 5,000 and 15,000 g/mol, or between 9,000 and 15,000 g/mol, or between 10,000 and 15,000 g/mol, or between about 11,000 and 14,000 g/mol, or between about 11,500 and 13,000 g/mol, or between about 12,000 and 13,000 g/mol, or between about 6,000 and 10,000 g/mol, or between about 7,000 and 9,000 g/mol, or between about 7,500 and 8,500 g/mol. In some embodiments, a poloxamer may have an average molecular weight between 9,500 and 15,000 g/mol. In some embodiments, a poloxamer may have an average molecular weight between 6,000 and 10,000 g/mol. In some embodiments, a poloxamer may have an average molecular weight between 12,000 and 18,000 g/mol. In some embodiments, a poloxamer may have an average molecular weight between 1,500 and 3,000 g/mol. In some embodiments, a poloxamer may have an average molecular weight between 6,000 and 9,000 g/mol. A skilled practitioner will understand that an average molecular weight described herein can be a number average molecular weight, a viscosity average molecular weight, or a weight average molecular weight. In some embodiments, polymers described herein (e.g., poloxamers and other polymers described herein) are characterized by weight average molecular weight. In some embodiments, polymers described herein (e.g., hyaluronic acid described herein) are characterized by viscosity average molecular weight, which in some embodiments can be determined by converting intrinsic viscosity measurement to average molecular weight, for example, using the Mark-Houwink equation.

In some embodiments, a poloxamer that may be included in a polymer combination preparation described herein may have polyoxypropylene with an average molecular weight between 1,000 and 5,000 g/mol, or between 1,500 and 4,500 g/mol.

In some embodiments, a poloxamer that may be included in a polymer combination preparation described herein may be a poloxamer variant. Examples of a poloxamer variant include, but are not limited to, poloxamines (e.g., amphiphilic block copolymers formed by four arms of poly(ethylene oxide)-poly(propylene oxide) (PEO-PPO) blocks bonded to a central ethylenediamine moiety), acrylate-modified poloxamer, thiol-modified poloxamer, and combinations thereof. See, e.g., Niu et al., J. Controlled Release, 2009, 137:49-56; and Alvarex-Lorenzo et al. “Poloxamine-based nanomaterials for drug delivery” Frontiers in Bioscience (2010), the contents of each of which are hereby incorporated by reference for at least their disclosure on modified poloxamers.

### B. Second Polymer Component Comprising One or More Exemplary Polymers That Are Not Poloxamers

In some embodiments, a polymer combination preparation described herein may comprise at least two polymer components, including, e.g., at least three, at least four, at least five, or more polymer components. In some embodiments, a second polymer component of a provided polymer combination preparation comprising poloxamer as a first polymer component at a concentration of 12.5% (w/w) or below may be or comprise at least one, including, e.g., at least two, at least three, at least four or more, biocompatible and/or biodegradable polymer components. Examples of such a biocompatible and/or biodegradable polymer component include, but are not limited to immunomodulatory polymers, carbohydrate polymers (e.g., a polymer that is or comprises a carbohydrate, e.g., a carbohydrate backbone, including, e.g., but not limited to chitosan, alginate, hyaluronic acid, and/or variants thereof), polyacrylic acid, silica gels, polyethylenimine (PEI), polyphosphazene, and/or variants thereof), cellulose, chitin, chondroitin sulfate, collagen, dextran, gelatin, ethylene-vinyl acetate (EVA), fibrin, poly(lactic-co-glycolic) acid (PLGA), polylactic acid (PLA), polyglycolic acid (PGA), polyethylene glycol (PEG), PEG diacrylate (PEGDA), disulfide-containing PEGDA (PEGSSDA), PEG dimethacrylate (PEGDMA), polydioxanone (PDO), polyhydroxybutyrate (PHB), poly(2-hydroxyethyl methacrylate) (pHEMA), polycarboxybetaine (PCB), polysulfobetaine (PSB), polycaprolactone (PCL), poly(beta-amino ester) (PBAE), poly(ester amide), poly(propylene glycol) (PPG), poly(aspartic acid), poly(glutamic acid), poly(propylene fumarate) (PPF), poly(sebacic anhydride) (PSA), poly(trimethylene carbonate) (PTMC), poly(desaminotyrosyltyrosine alkyl ester carbonate) (PDTE), poly[bis(trifluoroethoxy)phosphazene], polyoxymethylene, single-wall carbon nanotubes, polyanhydride, poly(N-vinyl-2-pyrrolidone) (PVP), poly(vinyl alcohol) (PVA), poly(acrylic acid) (PAA), poly(methacrylic acid) (PMA), polyacetal, poly(alpha ester), poly(ortho ester), polyphosphoester, polyurethane, polycarbonate, polyamide, polyhydroxyalkanoate, polyglycerol, polyglucuronic acid, starch, variants thereof, and/or combinations thereof.

In some embodiments, a second polymer component of a provided polymer combination preparation is or comprises a non-ionic polymer component. Examples of such a non-ionic polymer component include, but are not limited to polyvinyl alcohol (PVA), polyethylene oxide (PEO), and combinations thereof. In some embodiments, a second polymer component of a provided polymer combination preparation is or comprises a cationic polymer component, e.g., but not limited to chitosan, amino-containing polymers, collagen, gelatin, and combinations thereof. In some embodiments, a second polymer component of a provided polymer combination preparation is or comprises an anionic polymer component, examples of which may include, but are not limited to alginate, gellan gum, pectin, xanthan gum, carboxymethyl cellulose (CMC), polyacrylic acid, polyaspartic acid, and combinations thereof.

In some embodiments, a second polymer component of a provided polymer combination preparation is or comprises an immunomodulatory polymer, e.g., a polymer that modulates one or more aspects of an immune response (e.g., a polymer that induces innate immunity agonism). In some embodiments, an immunomodulatory polymer may be or comprise a polymer agonist of innate immunity as described in International Patent Application No. PCT/US20/31169 filed May 1, 2020, (published as WO2020/223698A1). In some embodiments, an immunomodulatory polymer may be or comprise a carbohydrate polymer (e.g., a polymer that is or comprises a carbohydrate, e.g., a carbohydrate backbone, including, e.g., but not limited to chitosan, alginate, hyaluronic acid, and/or variants thereof).

In some embodiments, a provided polymer combination preparation comprises at least one poloxamer at a concentration of 12.5% or below (e.g., 11% (w/w), 10.5% (w/w), 10% (w/w), 9% (w/w), 8% (w/w), 7% (w/w), 6% (w/w), or lower) and a second polymer component, which may be or comprise a carbohydrate polymer, e.g., a polymer that is or comprises a carbohydrate, e.g., a carbohydrate backbone, including, e.g., but not limited to hyaluronic acid, chitosan, and/or variants thereof.

### (I) Exemplary Hyaluronic Acid and Variants Thereof

In some embodiments, a carbohydrate polymer included in a provided polymer combination preparation comprising poloxamer is or comprises hyaluronic acid or a variant thereof. Hyaluronic acid (HA), also known as hyaluronan or hyaluronate, is a non-sulfated member of a class of polymers known as glycosaminoglycans (GAG) that is widely distributed in body tissues. HA is found as an extracellular matrix component of tissue that forms a pericellular coat on the surfaces of cells. In some embodiments, HA is a polysaccharide (which in some embodiments may be present as a salt, e.g., a sodium salt, a potassium salt, and/or a calcium salt) having a molecular formula of (C₁₄H₂₁NO₁₁)ₙ where n can vary according to the source, isolation procedure, and/or method of determination.

In some embodiments, HA that may be useful in accordance with the present disclosure can be isolated or derived from many natural sources. For example, in some embodiments, HA can be isolated or derived from, including, e.g., human umbilical cord, rooster combs, and/or connective matrices of vertebrate organisms. In some embodiments, HA can be isolated or derived from a capsular component of bacteria such as Streptococci. See, e.g., Kendall et al, (1937), Biochem. Biophys. Acta, 279, 401-405. In some embodiments, HA and/or variants thereof can be produced via microbial fermentation. In some embodiments, HA and/or variants thereof may be a recombinant HA or variants thereof, for example, produced using Gram-positive and/or Gram-negative bacteria as a host, including, e.g., but not limited to Bacillus sp., Lactococcos lactis, Agrobacterium sp., and/or Escherichia coli.

As discussed in the International Patent Application No. PCT/US20/31169 filed May 1, 2020 (published as WO2020/223698A1), biological activities of HA differ, depending on its molecular weight - for example, high molecular weight HA (high MW HA) can possess anti-inflammatory or immunosuppressive activities, while low molecular weight HA (low MW HA) may exhibit pro-inflammatory or immunostimulatory behaviors. See, e.g., Gao et al. “A low molecular weight hyaluronic acid derivative accelerates excisional wound healing by modulating pro-inflammation, promoting epithelialization and neovascularization, and remodeling collagen” Int. J. Mol Sci (2019) 20:3722; Cyphert et al. “Size Matters: Molecular Weight Specificity of Hyaluronan Effects in Cell Biology.” Int. J. Cell Biol. (2015) 2015: 563818; Dicker et al. “Hyaluronan: A simple polysaccharide with diverse biological functions” Acta Biomater. (2014) 10:1558-1570; Aya and Stern “Hyaluronan in wound healing: Rediscovering a major player.” Wound Repair Regen. (2014) 22:579-593; and Frenkel “The role of hyaluronan in wound healing” Int. Wound J. (2014) 11:159-163, the entire contents of each of which are incorporated herein by reference in their entirety for the purposes described herein. Accordingly, in some embodiments, HA or variants thereof that may be included in a provided polymer combination preparation can have a low molecular weight, for example, an average molecular weight of 500 kDa or less, including, e.g., 450 kDa, 400 kDa, 350 kDa, 300 kDa, 250 kDa, 200 kDa, 150 kDa, 100 kDa, 50 kDa, or less. In some embodiments, HA or variants thereof that may be included in a provided polymer combination preparation may have an average molecular weight of about 100 kDa to about 200 kDa. In some embodiments, HA or variants thereof that may be included in a provided polymer combination preparation may have an average molecular weight of about 100 kDa to about 150 kDa. In some embodiments, HA or variants thereof that may be included in a provided polymer combination preparation may have an average molecular weight of about 250 kDa to about 350 kDa. In some embodiments, HA or variants thereof that may be included in a provided polymer combination preparation may have an average molecular weight of about 300 kDa to about 400 kDa. In some embodiments, a polymer combination preparation described herein may comprise a poloxamer (e.g., ones described herein) and low molecular weight HA or variants thereof in the absence of an immunomodulatory payload may be useful for inducing innate immunity agonism.

In some embodiments, HA or variants thereof that may be included in a provided polymer combination preparation can have a high molecular weight, for example, an average molecular weight of greater than 500 kDa or higher, including, e.g., 550 kDa, 600 kDa, 650 kDa, 700 kDa, 750 kDa, 800 kDa, 850 kDa, 900 kDa, 950 kDa, 1 MDa, 1.1 MDa, 1.2 MDa, 1.3 MDa, 1.4 MDa, 1.5 MDa, 1.6 MDa, 1.7 MDa, 1.8 MDa, 1.9 MDa, 2 MDa, 2.5 MDa, 3 MDa, 3.5 MDa, 4 MDa, 4.5 MDa, or higher. In some embodiments, HA or variants thereof that may be useful in accordance with the present disclosure may have an average molecular weight of about 600 kDa to about 900 kDa. In some embodiments, HA or variants thereof that may be useful in accordance with the present disclosure may have an average molecular weight of about 700 kDa to about 900 kDa. In some embodiments, HA or variants thereof that may be included in a provided polymer combination preparation may have an average molecular weight of about 500 kDa to about 800 kDa. In some embodiments, HA or variants thereof that may be included in a provided polymer combination preparation may have an average molecular weight of about 600 kDa to about 800 kDa. In some embodiments, HA or variants thereof that may be included in a provided polymer combination preparation may have an average molecular weight of about 700 kDa to about 800 kDa. In some embodiments, HA or variants thereof that may be useful in accordance with the present disclosure may have an average molecular weight of about 1 MDa to about 3 MDa. In some embodiments, a polymer combination preparation described herein may comprise a poloxamer (e.g., ones described herein) and high molecular weight HA or variants thereof in the absence of an immunomodulatory payload may be useful for resolving inflammation (e.g., immunosuppressive inflammation).

In some embodiments, a provided polymer combination preparation comprises a hyaluronic acid variant. In some embodiments, a hyaluronic acid variant is water-soluble. In some embodiments, a hyaluronic acid variant may be a chemically modified hyaluronic acid, e.g., in some embodiments, hyaluronic acid is esterified. Examples of chemical modifications to hyaluronic acid include, but are not limited to, addition of thiol, haloacetate, butanediol, diglycidyl, ether, dihydrazide, aldehyde, glycan, and/or tyramine functional groups. Additional hyaluronic acid modifications and variants are known in the art. See e.g., Highley et al., “Recent advances in hyaluronic acid hydrogels for biomedical applications” Curr Opin Biotechnol (2016) Aug 40:35-40; Burdick & Prestwich, “Hyaluronic acid hydrogels for biomedical applications” Advanced Materials (2011); Prestwhich, “Hyaluronic acid-based clinical biomaterials derived for cell and molecule delivery in regenerative medicine” J. Control Release (2011) Oct 30; 155(2): 193-199; each of which are incorporated herein by reference in their entirety for the purposes described herein.

In some embodiments, a provided polymer combination preparation comprises at least one poloxamer present at a concentration of 12.5% (w/w) or below and a second polymer component, which may be or comprise hyaluronic acid or variant thereof. In some such embodiments, HA or a variant thereof may be present in a provided polymer combination preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), or 1% (w/w) or lower. In some embodiments, HA or a variant thereof may be present in a provided polymer combination preparation at a concentration of about 0.5 % (w/w) to about 5% (w/w), e.g., at a concentration of 0.5% (w/w), 0.6% (w/w), 0.7% (w/w), 0.8% (w/w), 0.9% (w/w), 1% (w/w), 1.5% (w/w), 2% (w/w), 2.5% (w/w), 3% (w/w), 3.5% (w/w), 4% (w/w), 4.5% (w/w), or 5% (w/w). In some embodiments, HA or a variant thereof having a low molecular weight (e.g., as described herein) may be present in a provided polymer combination preparation at a concentration of at least about 1.5% (w/w) or higher, including, e.g., at least 2% (w/w), at least 2.5% (w/w), at least 3% (w/w), at least 4% (w/w), at least 5% (w/w), at least 6% (w/w), at least 7% (w/w), at least 8% (w/w), at least 9% (w/w), or higher. In some embodiments, HA or a variant thereof having a low molecular weight (e.g., as described herein) may be present in a provided polymer combination preparation at a concentration of about 1.5% (w/w) to about 5% (w/w). In some embodiments, HA or a variant thereof having a low molecular weight (e.g., as described herein) may be present in a provided polymer combination preparation at a concentration of about 0.5% (w/w) to about 10% (w/w). In some embodiments, HA or a variant thereof having a low molecular weight (e.g., as described herein) may be present in a provided polymer combination preparation at a concentration of about 1% (w/w) to about 10% (w/w) or about 1.5% (w/w) to about 10% (w/w). In some embodiments, HA or a variant thereof having a low molecular weight (e.g., as described herein) may be present in a provided polymer combination preparation at a concentration of about 0.7% (w/w) to about 4% (w/w) or about 1.5% (w/w) to about 4% (w/w). In some embodiments, HA or a variant thereof having a low molecular weight (e.g., as described herein) may be present in a provided polymer combination preparation at a concentration of about 3% (w/w) to about 7% (w/w). In some embodiments, HA or a variant thereof having a high molecular weight (e.g., as described herein) may be present in a provided polymer combination preparation at a concentration of 2% (w/w) or lower, including, e.g., 1.5% (w/w), 1.25% (w/w), 1% (w/w), or lower. In some embodiments, HA or a variant thereof having a high molecular weight (e.g., as described herein) may be present in a provided polymer combination preparation at a concentration of about 0.5% (w/w) to about 3% (w/w).

### (II) Exemplary Chitosan and Variants Thereof

In some embodiments, a carbohydrate polymer included in a provided polymer combination preparation comprising poloxamer (e.g., as described herein) may be or comprise chitosan or a variant thereof. Examples of chitosan and/or variants thereof that can be included in a polymer combination preparation described herein include, but are not limited to chitosan, chitosan salts (e.g., chitosan HCl, chitosan chloride, chitosan lactate, chitosan acetate, chitosan glutamate), alkyl chitosan, aromatic chitosan, carboxyalkyl chitosan (e.g., carboxymethyl chitosan), hydroxyalkyl chitosan (e.g., hydroxypropyl chitosan, hydroxyethyl chitosan), aminoalkyl chitosan, acylated chitosan, phosphorylated chitosan, thiolated chitosan, quaternary ammonium chitosan (e.g., N-(2-hydroxyl) propyl-3-trimethyl ammonium chitosan chloride), guanidinyl chitosan, chitosan oligosaccharide, glycated chitosan (e.g., N-dihydrogalactochitosan), chitosan poly(sulfonamides), chitosan-phenylsuccinic acid (e.g., products formed from the reaction of phenylsuccinic anhydride or a variant thereof (including, e.g., 2-phenylsuccinic anhydride, 2-phenylsuccinic acid derivatives, 2-O-acetyl L-Malic anhydride, etc.) and chitosan (e.g., Chitosan Phenylsuccinic acid hemi-amide - ring opened amide-carboxylic acid derivative), and variants or combinations thereof. In some embodiments, a carbohydrate polymer included in a provided polymer combination preparation comprising poloxamer (e.g., as described herein) may be or comprise carboalkyl chitosan (e.g., carboxymethyl chitosan).

Those skilled in the art will appreciate that, in some cases, chitosan and/or variants thereof can be produced by deacetylation of chitin. In some embodiments, chitosan or variants thereof included in a polymer combination preparation comprising poloxamer (e.g., as described herein) is characterized by degree of deacetylation (i.e., percent of acetyl groups removed) of at least 70% or above, including, e.g., at least 75%, at least 80%, at least 85%, at least 90%, at least 95%, or higher (including up to 100%). In some embodiments, a chitosan or variants thereof is characterized by degree of deacetylation of no more than 99%, no more than 95%, no more than 90%, no more than 85%, no more than 80%, no more than 75% or lower. Combinations of the above-mentioned ranges are also possible. For example, a chitosan or variants thereof may be characterized by degree of deacetylation of 80%-95%, 70%-95%, or 75%-90%. As will be recognized by one of those skilled in the art, degree of deacetylation (%DA) can be determined by various methods known in the art, e.g., in some cases, by NMR spectroscopy.

In some embodiments, chitosan or variants thereof included in a polymer combination preparation comprising poloxamer (e.g., as described herein) may have an average molecular weight of at least 5 kDa or higher, including, e.g., at least 10 kDa or higher, including, e.g., at least 20 kDa, at least 30 kDa, at least 40 kDa, at least 50 kDa, at least 60 kDa, at least 70 kDa, at least 80 kDa, at least 90 kDa, at least 100 kDa, at least 110 kDa, at least 120 kDa, at least 130 kDa, at least 140 kDa, at least 150 kDa, at least 160 kDa, at least 170 kDa, at least 180 kDa, at least 190 kDa, at least 200 kDa, at least 210 kDa, at least 220 kDa, at least 230 kDa, at least 240 kDa, at least 250 kDa, at least 260 kDa, at least 270 kDa, at least 280 kDa, at least 290 kDa, at least 300 kDa, at least 350 kDa, at least 400 kDa, at least 500 kDa, at least 600 kDa, at least 700 kDa, or higher. In some embodiments, chitosan or variants thereof included in a polymer combination preparation comprising poloxamer (e.g., as described herein) may have an average molecular weight of no more than 750 kDa or lower, including, e.g., no more than 700 kDa, no more than 600 kDa, no more than 500 kDa, no more than 400 kDa, no more than 300 kDa, no more than 200 kDa, no more than 100 kDa, no more than 50 kDa, or lower. Combinations of the above-mentioned ranges are also possible. For example, in some embodiments, chitosan or variants thereof included in a polymer combination preparation comprising poloxamer (e.g., as described herein) is characterized by an average molecular weight of 10 kDa to 700 kDa, or 20 kDa to 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a polymer combination preparation comprising poloxamer (e.g., as described herein) is characterized by an average molecular weight of 20 kDa to 700 kDa, or 30 kDa to 500 kDa. As noted herein, an average molecular weight may be a number average molecular weight, weight average molecular weight, or peak average molecular weight.

In some embodiments, chitosan or variants thereof included in a polymer combination preparation comprising poloxamer (e.g., as described herein) is characterized by a molecular weight distribution in a range of 10 kDa to 700 kDa, or 20 kDa or 700 kDa, or 30 kDa to 500 kDa, or 150 kDa to 600 kDa, or 150 kDa to 400 kDa, or 50 kDa to 150 kDa, or 10 kDa to 50 kDa. In some embodiments, chitosan or variants thereof included in a polymer combination preparation comprising poloxamer (e.g., as described herein) is characterized by a molecular weight distribution in a range of 20 kDa to 700 kDa, or 30 kDa to 500 kDa.

In some embodiments, chitosan or variants thereof included in a polymer combination preparation comprising poloxamer (e.g., as described herein) may be characterized by a viscosity of no more than 3,500 mPa·s or lower, including, e.g., no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, chitosan or variants thereof may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, or higher. Combinations of the above-mentioned ranges are also possible. For example, in some embodiments, such a viscous polymer solution of or comprising chitosan or variants thereof may be characterized by a viscosity of 5 mPa·s to 3,000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of chitosan or variants thereof described herein is measured at 1% in 1% acetic acid at 20° C.

In some embodiments, a polymer combination preparation comprising poloxamer (e.g., as described herein) comprises at least one or more (e.g., 1, 2, 3 or more) chitosan and/or variants thereof (including, e.g., modified chitosan and/or salts of chitosan or modified chitosan such as a chloride salt or a glutamate salt). For example, in some embodiments, chitosan and/or variants thereof (including, e.g., modified chitosan and/or salts of chitosan or modified chitosan such as a chloride salt or a glutamate salt) may be characterized by degree of deacetylation of 70%-95%, or 75%-90%, or 80%-95%, or greater than 90%. In some embodiments, chitosan and/or variants thereof (including, e.g., modified chitosan and/or salts of chitosan or modified chitosan such as a chloride salt or a glutamate salt) may be characterized by an average molecular weight of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., modified chitosan and/or salts of chitosan or modified chitosan such as a chloride salt or a glutamate salt) may be characterized by a molecular weight distribution in the range of 10 kDa to 700 kDa, 20 kDa to 600 kDa, 30 kDa to 500 kDa, 150 kDa to 400 kDa, or 200 kDa to 600 kDa (e.g., measured as chitosan or chitosan salt, e.g., chitosan acetate). In some embodiments, chitosan and/or variants thereof (including, e.g., salts thereof such as a chloride salt or a glutamate salt) may be characterized by a viscosity ranging from 5 to 3,000 mPa·s, or 5 to 300 mPa·s, or 20 to 200 mPa·s. In some embodiments, such chitosan and/or variants thereof (including, e.g., salts thereof such as a chloride salt or a glutamate salt) may be or comprise PROTASAN® UltraPure chitosan chloride and/or chitosan glutamate salt (e.g., obtained from NovoMatrix®, which is a business unit of FMC Health and Nutrition (now a part of Du Pont; Product No. CL 113, CL 114, CL 213, CL 214, G 113, G 213, G 214). In some embodiments, such chitosan and/or variants thereof (including, e.g., salts thereof such as a chloride salt or a glutamate salt) may be or comprise chitosan, chitosan oligomers, and/or variants thereof (including, e.g., Chitosan HCl, carboxymethyl chitosan, chitosan lactate, chitosan acetate), e.g., obtained from Heppe Medical Chitosan GMBH (e.g., Chitoceuticals® or Chitoscience®).

In some embodiments, chitosan or variants thereof included in a polymer combination preparation comprising poloxamer (e.g., as described herein) is or comprises carboxyalkyl chitosan (e.g., carboxymethyl chitosan) that is characterized by at least one or all of the following characteristics: (1) degree of deacetylation of 80%-95%; (ii) an average molecular weight of 30 kDa to 500 kDa; or a molecular weight distribution of 30 kDa to 500 kDa; and (iii) a viscosity ranging from 5 to 300 mPa·s.

In some embodiments, chitosan or variants thereof included in a polymer combination preparation comprising poloxamer (e.g., as described herein) is or comprises a variant of chitosan (e.g., as described herein). In some embodiments, such a variant of chitosan may include chemical modification(s) of one or more chemical moieties, e.g., hydroxyl and/or amino groups, of the chitosan chains. In some embodiments, such a variant of chitosan is or comprises a modified chitosan such as, e.g., but not limited to a glycated chitosan (e.g., chitosan modified by addition of one or more monosaccharide or oligosaccharide side chains to one or more of its free amino groups). Exemplary glycated chitosan that are useful herein include, e.g., but are not limited to ones described in US 5,747,475, US 6,756,363, WO 2013/109732, US 2018/0312611, and US 2019/0002594, the contents of each of which are incorporated herein by reference for the purposes described herein.

In some embodiments, chitosan or variants thereof included in a polymer combination preparation comprising poloxamer (e.g., as described herein) is or comprises chitosan conjugated with a polymer that increases its solubility in aqueous environment (e.g., a hydrophilic polymer such as polyethylene glycol).

In some embodiments, chitosan or variants thereof included in a polymer combination preparation comprising poloxamer (e.g., as described herein) is or comprises thiolated chitosan. Various modifications to chitosans, e.g., but not limited to carboxylation, PEGylation, galactosylation (or other glycations), and/or thiolation are known in the art, e.g., as described in Ahmadi et al. Res Pharm Sci., 10(1): 1-16 (2015), the contents of which are incorporated herein by reference for the purposes described herein. Those skilled in the art reading the present disclosure will appreciate that other modified chitosans can be useful for a particular application in which a method is being practiced.

In some embodiments, a provided polymer combination preparation comprises at least one poloxamer present at a concentration of 12.5% or below and a second polymer component, which may be or comprise chitosan or variant thereof. In some such embodiments, chitosan or a variant thereof may be present in a provided polymer combination preparation at a concentration of about 10% (w/w) or lower, including, e.g., 9% (w/w), 8% (w/w), 7% (w/w), 6% (w/w), 5% (w/w), 4% (w/w), 3% (w/w), 2% (w/w), 1% (w/w), 0.5% (w/w), 0.4% (w/w), 0.3% (w/w), 0.2% (w/w), 0.1% (w/w) or lower. In some embodiments, chitosan or a variant thereof may be present in a provided polymer combination preparation at a concentration of 0.1% (w/w) to 10% (w/w), or 0.1% (w/w) to 8% (w/w), or 0.1% (w/w) to 5% (w/w), or 1% (w/w) to 5% (w/w), or about 1% (w/w) to about 3% (w/w).

### C. Exemplary Payloads (e.g., Therapeutic Agents)

In some embodiments, biomaterial preparations (e.g., provided polymer combination preparations) may be administered without an additional payload; in some embodiments, such preparations may themselves have certain immunomodulatory properties. Alternatively or additionally, in some embodiments, biomaterial preparations (e.g., polymer combination preparations) may comprise and/or otherwise be administered in combination with one or more payload agents (e.g., therapeutic agents, e.g., immunomodulatory payloads, e.g., immunomodulatory agents) That is, in some embodiments, an immunomodulatory composition may comprise or consist of a polymer combination preparation and one or more payload agents.

In some embodiments, a payload is or comprises a therapeutic agent.

In some embodiments, a payload is or comprises an immunomodulatory agent.

In some embodiments, a payload is an agent (e.g., a therapeutic agent) approved by the Food and Drug Administration (e.g., as described in Zhong et al., “A comprehensive Map of FDA-Approved Pharmaceutical Products.” Pharmaceutics, 2019 Dec; 10(4): 263, the contents of which are incorporated herein by reference for purposes described herein).

In some embodiments, a payload is an agent (e.g., a therapeutic agent) that inhibits or reduces level (e.g., expression and/or activity) of a target that is drugged by an agent (e.g., a therapeutic agent) approved by the Food and Drug Administration (e.g., as described in Zhong et al., “A comprehensive Map of FDA-Approved Pharmaceutical Products.” Pharmaceutics, 2019 Dec; 10(4): 263, the contents of which are incorporated herein by reference for purposes described herein).

In some embodiments, a payload is an agent (e.g., a therapeutic agent) that induces or increases level (e.g., expression and/or activity) of a target that is drugged by an agent (e.g., a therapeutic agent) approved by the Food and Drug Administration (e.g., as described in Zhong et al., “A comprehensive Map of FDA-Approved Pharmaceutical Products.” Pharmaceutics, 2019 Dec; 10(4): 263, the contents of which are incorporated herein by reference for purposes described herein).

In some embodiments, a payload is not a toxic (e.g., a cytotoxic or cytostatic, or other antiproliferative) agent, e.g., it is not a traditional chemotherapeutic agent that acts simply by killing cancer cells, but does not promote a clinically relevant extent of immunogenic cell death (for example, see: Vacchelli et al., “Trial watch: Chemotherapy with immunogenic cell death inducers”, Oncoimmunology, Mar. 1, 2013; Kepp et al., “Consensus guidelines for the detection of immunogenic cell death” Oncoimmunology, Dec. 13, 2014; Bloy et al., “Immunogenic stress and death of cancer cells: Contribution of antigenicity vs adjuvanticity to immunosurveillance” Immunology Reviews, November 2017; Michaud et al., “Autophagy-dependent anticancer immune responses induced by chemotherapeutic agents in mice”, Science, Dec. 16, 2011; Galluzzi et al., “Molecular mechanisms of cell death: recommendations of the Nomenclature Committee on Cell Death 2018”, Cell Death Differentiation, March 2018; Galluzzi et al., “Immunogenic cell death in cancer and infectious disease”, Nature Reviews Immunology, October 2016; Galluzzi et al., “Immunostimulation with chemotherapy in the era of immune checkpoint inhibitors”, Nature Reviews Clinical Oncology, Aug. 5, 2020; the contents of each of which are incorporated herein by reference for purposes described herein). In some embodiments, whether such a chemotherapeutic agent can promote a clinically relevant extent of immunogenic cell death can be determined, for example, by assessing for relative therapeutic benefit of the chemotherapeutic agent following treatment of the same tumor model in immunocompromised versus healthy mice. Examples of a traditional chemotherapeutic agent can be found among any of a variety of classes of anti-cancer agents including, but not limited to, alkylating agents, anti-metabolites, topoisomerase inhibitors, and/or mitotic inhibitors. In some embodiments, a polymer combination preparation composition as described herein is substantially free of any traditional chemotherapeutic agent. In some embodiments, a polymer combination preparation composition is substantially free of a cytotoxic or cytostatic agent (or other antiproliferative agent).

In some embodiments, such a payload may be dispersed within a biomaterial preparation (e.g., a polymer combination preparation described herein). In some embodiments, the present disclosure, among other things, provides a composition comprising a polymer combination preparation and/or one or more payloads, wherein at least some of the payload(s) is dispersed within the polymer combination preparation. Examples of payloads include, but are not limited to nucleic acids, polypeptides, peptides, small molecules, lipids, saccharides, metals, or combination or complex thereof.

In some embodiments, a payload that may be included in a biomaterial preparation (e.g., a provided polymer combination preparation) may be or comprise a therapeutic agent for treatment and/or prophylaxis of a disease, disorder, or condition. In some embodiments, a therapeutic agent included in a biomaterial preparation (e.g., a provided polymer combination preparation) may be or comprise an agent for immunomodulation, wound healing, cancer therapy, and/or analgesia. In some embodiments, a therapeutic agent included in a biomaterial preparation (e.g., a provided polymer combination preparation) may be useful for treatment of cancer. In some embodiments, a payload that may be included in a biomaterial preparation (e.g., a provided polymer combination preparation) is or comprises a chemotherapeutic agent, for example, in some embodiments a chemotherapeutic agent that induces immunogenic cell death. As will be recognized by one of ordinary skill in the art, a chemotherapeutic agent suitable for use in accordance with the present disclosure may be a synthetic or natural compound; a single molecule or a complex of different molecules. In some embodiments, suitable chemotherapeutic agents that induces immunogenic cell death can belong to any of various classes of compounds including, but not limited to, small molecules, peptides, saccharides, steroids, antibodies, fusion proteins, nucleic acid agents (e.g., but not limited to antisense polynucleotides, ribozymes, and small interfering RNAs), peptidomimetics, and the like. Similarly, suitable chemotherapeutic agents can be found among any of a variety of classes of anti-cancer agents including, but not limited to, alkylating agents, anti-metabolites, topoisomerase inhibitors, and/or mitotic inhibitors.

In some embodiments, a payload that may be included in a biomaterial preparation (e.g., a provided polymer combination preparation) is or comprises one or more nucleic acid agents. Such a nucleic acid agent may have enzymatic activity (e.g., ribozyme activity), gene expression inhibitory activity (e.g., as an antisense or interfering RNA agent, etc.), polypeptide-encoding activity, immunomodulatory activity, and/or other activities. In some embodiments, a nucleic acid agent that may be included in a biomaterial preparation (e.g., a provided polymer combination preparation) may itself act to modulate one or more aspects of an immune response, or may encode a modulator of one or more aspects of an immune response.

In certain embodiments, a payload that may be included in a biomaterial preparation (e.g., a provided polymer combination preparation) is or comprises an antibiotic. Examples of antibiotics include but are not limited to: Afabicin (Debio 1450), Amikacin, Amoxicillin, Ampicillin, Amprolium, Apramycin (EBL-1003), ARV-1801 (sodium fusidate), Azithromycin, Bacitracin, Benapenem, BOS-228, Brilacidin, BV100, Cefaclor, Cefdinir, Cefepime, Cefilavancin, Cefotaxime, Ceftazidime, Ceftibuten, Ceftriaxone, Cefuroxime, CG-549, Chlortetracycline, Cilastatin, Ciprofloxacin, Clarithromycin, Clavulanate, Clindamycin, Clopidol, Contezolid (MRX-I)/contezolid acefosamil (MRX-4), CRS3123, Dalbavancin, Decoquinate, Delpazolid (LCB01-0371), Demeclocycline, Diclazuril, Dicloxacillin, DNV3837/DNV3681, Doripenem, Doxycycline, Durlobactam, EMROK/EMROK O, Enmetazobactam, Eravacycline, Ertapenem, Erythromycin, ETX0282CPDP/ ETX1317, Fenbendazole, Finafloxacin, Gentamicin, Gepotidacin (GSK2140944), Halifuginone, Hygromycin B, Ibezapolstat, Imipenem, KBP-7072, Laidlomycin, Lasalocid, Levofloxacin, Lincomycin, Lubabegron, Melengestrol, Melengestrol Acetate, Meropenem, MGB-BP-3, Minocycline, Monensin, Moxifloxacin, MRX-8, Nacubactam (OP0595), Nafithromycin (WCK 4873), Neomycin, Omadacycline, OMNIvance (QPX7728), Oritavancin, Ormetoprim, Oxacillin, Oxytetracycline, Penicillin V potassium, Pyrantel, Ractopamine, Ridinilazole, Robenidine, Salinomycin, Semduramicin, SPR206, SPR741, Sulbactam, Sulfadimethoxine, Sulfamethazine, Sulfamethoxazole, Sulfaquinoxaline, Sulfasalazine, Sulfisoxazole, Sulopenem/sulopenem etzadroxil-probenecid, T-4288 (solithromycin), Taigexyn (nemonoxacin), Taniborbactam, Tebipenem/tebipenem pivoxil hydrobromide, Telavancin, Tetracycline, TNP-2092, Tobramycin, TP-271, TP-6076, Trimethoprim, TXA709/TXA707, Tylosin, Vancomycin, Virginiamycin, VNRX-7145, XNW4107, Zevtera (ceftobiprole), Zidebactam, Zilpaterol, Zoalene, Zoliflodacin (ETX0914), and combinations thereof.

In certain embodiments, a payload that may be included in a biomaterial preparation (e.g., a provided polymer combination preparation) is or comprises an antibody. Examples of an antibody include but are not limited to: Adalimumab, Alemtuzumab, Alirocumab, Atezolizumab, Avelumab, Belimumab, Benralizumab, Bevacizumab, Bezlotoxumab, Blinatumomab, Brentuximab vedotin, Brodalumab, Brolucizumab, Burosumab, Canakinumab, Cemiplimab, Certolizumab, Cetuximab, Daratumumab, Denosumab, Dinutuximab, Dupilumab, Durvalumab, Elotuzumab, Emapalumab, Emicizumab, Erenumab, Evolocumab, Fremanezumab, Galcanezumab, Gemtuzumab zogamicin, Golimumab, Guselkumab, Ibalizumab, Ibritumomab tiuxetan, Idarucizumab, Infliximab, Inotuzumab ozogamicin, Ipilimumab, Ixekizumab, Lanadelumab, Mepolizumab, Mogamulizumab, Moxetumomab, Natalizumab, Necitumumab, Nivolumab, Obiltoxaximab, Obinutuzumab, Ocrelizumab, Ofatumumab, Olaratumab, Omalizumab, Palivizumab, Panitumumab, Pembrolizumab, Pertuzumab, Polatuzumab, Ramucirumab, Ranibizumab, Ravulizumab, Reslizumab, Risankizumab, Rituximab, Romosozumab, Sarilumab, Secukinumab, Siltuximab, Tildrakizumab, Tocilizumab, Trastuzumab, Trastuzumab, Ustekinumab, Vedolizumab, and combinations thereof (see e.g., Lu et al., Development of therapeutic antibodies for the treatment of diseases. Journal of Biomedical Science, 2020).

In certain embodiments, a payload that may be included in a biomaterial preparation (e.g., a provided polymer combination preparation) is or comprises an analgesic. Examples of various types of analgesics include but are not limited to: Anticonvulsants, Antidepressants, Anxiolytics, Corticosteroids, COX-2 inhibitors, Fibromyalgia medications, Mixed opioid agonist/antagonists, Muscle relaxants, Nonsteroidal anti-inflammatory drugs (NSAIDs), Opioid analgesics, or combinations thereof. In some embodiments, an analgesic that may be included in a biomaterial preparation (e.g., a provided polymer combination preparation) is or comprises: Acetaminophen, Acetaminophen with codeine, Alprazolam, Amitriptyline, Aspirin, Baclofen, Buprenorphine, Bupropion, Butorphanol, Carbamazepine, Carisoprodol, Celecoxib, Chlorzoxazone, Clonazepam, Cortisone, Cyclobenzaprine, Dantrolene, Desipramine, Dexamethasone, Diazepam, Diclofenac, Diflunisal, Duloxetine, Etodolac, Fenoprofen, Fentany, Fluoxetine, Flurbiprofen, Gabapentin, Hydrocodone, Hydrocodone with ibuprofen, Hydrocodone with acetaminophen, Hydromorphone, Ibuprofen, Imipramine, Indomethacin, Ketoprofen, Ketorolac, Lamotrigine, Lorazepam, Mefenamic acid, Meloxicam, Meperidine, Metaxalone, Methadone, Methocarbamol, Methylprednisolone, Milnacipran, Morphine, Nabumetone, Nalbuphine, Naproxen, Orphenadrine, Oxaprozin, Oxycodone, Oxycodone with acetaminophen, Oxycodone with aspirin, Oxycodone with ibuprofen, Oxymorphone, Pentazocine, Pentazocine/naloxone, Piroxicam, Prednisolone, Prednisone, Pregabalin, Propoxyphene with acetaminophen, Propoxyphene with aspirin, Rofecoxib, Sulindac, Tapentadol, Tapentadol ER, Tiagabine, Tizanidine, Tolmetin, Topiramate, Tramadol, Tramadol hydrochloride, Tramadol with acetaminophen, Triamcinolone, Triazolam, Valdecoxib, Venlafaxine, or combinations thereof.

In certain embodiments, a payload that may be included in a biomaterial preparation (e.g., a provided polymer combination preparation) is or comprises an anticoagulant. In some embodiments, an anticoagulant that may be included in a biomaterial preparation (e.g., a provided polymer combination preparation) is or comprises: Apixaban, Betrixaban, Dabigatran, Dalteparin sodium, Darexaban, Edoxaban, Eribaxaban, Letaxaban, Rivaroxaban, Warfarin, or combinations thereof.

In certain embodiments, a payload that may be included in a biomaterial preparation (e.g., a provided polymer combination preparation) is or comprises a coagulant. In some embodiments, a coagulant that may be included in a biomaterial preparation (e.g., a provided polymer combination preparation) is or comprises: Coagulation Factor VIIa (e.g., recombinant Coagulation Factor VIIa, e.g., NovoSeven, or NovoSevenRT), Coagulation Factor IX (e.g., recombinant Coagulation Factor IX, e.g., Alprolix, Benefix, Ixinity, or Rixubis), Coagulation Factor IX fused to Albumin (e.g., recombinant Coagulation Factor IX fused to albumin, e.g., Idelvion), GlycoPEGylated Coagulation Factor IX (e.g., glycoPEGylated recombinant Coagulation Factor IX, e.g., Rebinyn), Coagulation Factor XIII A-Subunit (e.g., recombinant Coagulation Factor XIII A-subunit, e.g., Tretten), Coagulation Factor X (e.g., recombinant Coagulation Factor X), or combinations thereof.

In certain embodiments, a payload that may be included in a biomaterial preparation (e.g., a provided polymer combination preparation) is or comprises an antiemetic. Examples of various types of antiemetics include but are not limited to: Anticholinergics, Cannabinoids, Corticosteroids, Dopamine receptor antagonists, H-1 antihistamines, Neurokinin-1 inhibitors, Serotonin receptor antagonists, or combinations thereof. In some embodiments, an antiemetic that may be included in a biomaterial preparation (e.g., a provided polymer combination preparation) is or comprises: Aprepitant, Bismuth subsalicylate, Cyclizine, Dexamethasone, Dimenhydrinate, Diphenhydramine, Dolasetron, Doxylamine-pyridoxine, Dronabinol, Droperidol, Granisetron, Lorazepam, Meclizine, Methylprednisolone, Metoclopramide, Nabilone, Netupitant-palonosteron, Ondansetron, Orthophosphoric acid, Palonosetron, Prochlorperazine, Prochlorperazine maleate, Promethazine, Pyridoxine, Rolapitant, Scopolamine, or combinations thereof.

In certain embodiments, a payload that may be included in a biomaterial preparation (e.g., a provided polymer combination preparation) is or comprises an agent that promotes wound healing. In some such embodiments, an agent that promotes wound healing is or comprises: Acesulfame K, Acetamide MEA (monoethanolamine), Acetic acid, Activated charcoal, African palm oils, Alcohol, Allantoin, Almond meal, Aloe vera, Aluminum hydroxide, Aluminum magnesium hydroxide stearate, Aluminum oxide, Aluminum pigment, Aluminum sulfate, Ammonium phosphate, Angelica sp., Aqueous wheat extract, Arachidyl alcohol, Ascorbyl palmitate (Vitamin C ester), Ascorbyl tetraisopalmitate (Vitamin C ester), Avocado oil, Bacitracin, Beeswax, Behenyl alcohol (docosanol, Abreva), Benzalkonium chloride, Benzocaine, Benzoic acid, Benzyl alcohol, Betaines (various forms), Bisabolol (chamomile oil), Bismuth subgallate, Bismuth tribromophenate, Borneol, Butylated Hydroxytoluene (BHT), Butylene glycol, Butyrospermum parkii, Cadexomer iodine, Calamine, Calcium, Calcium carbonate, Calcium chloride, Calcium oxide, Calcium sulfate, Camella sinensis, Candelilla wax, Capryloyl glycine, Carvacrol, Centella asiatica, Ceramide, Ceteareth-10 phosphate, Cetearyl alcohol (Cetostearyl alcohol), Ceteth-20, Cetyl alcohol, Cetyl dimethicone copolyol, Cetyl palmitate, Chlorhexidine, Chlorine dioxide, Chlorophyllin copper complex sodium, Cholesterol, Chromium chloride, Citric acid, Citris grandis extract, Cobalt chloride, Cocoamphodiacetate, Colloidal silica, Conjugated linoleic acid, Copper, Copper chloride (cupric chloride), Crystal violet, Cupuacu butter, Cyclodextrin, Cyclomethicone, DEA Cetyl phosphate, Decanoic acid (capric acid), Dehydroacetic acid, Dialkyl carbamoyl chloride, Diazolidinyl urea, Dicetyl phosphate, Diisopropyl adipate, Dimethicone, Dipolyhydroxystearat e, Dissolved oxygen, DMDM hydantoin, EDTA, Ethanol, Ethoxydiglycol, Ethylene glycol monostearate, Ethylhexyl glycerin, Ethylhexyl palmitate, Eucalyptus oil, Eugenol, Extracts of licorice (deglycyrrhizinated), Ferric chloride Hexahydrate, Ferric oxide, Fruit extract, Fumed silica, Gentian violet, Germaben II, Glycerin (glycerol), Glyceryl monolaurate, Glyceryl monostearate, Glyceryl stearate, Glycyrrhetinic acid (licorice extract), Guar gum (Cyaiuopsis letragonolobus), Gum mastic, Hectorite clay, Hexyl laurate, Hydrochloric acid, Hydrocortisone, Hydrogen peroxide, Hydrogenated castor oil, Hydrogenated lecithin, Hydroquinone, Hydrous lanolin, Hydroxypropyl bispalmitamide MEA (ceramide), Hydroxypropyl guar, Hypochlorous acid, Iodine, Iodoform, Iron (various forms), Iron sulfate, Isohexadecane, Isopropyl alcohol, Isopropyl myristate, Isopropyl sorbate, Kaolin, Karaya gum, Keratin, Konjac flour, Lactic acid, Lavender, Lecithin, Lemon, L-glutamic acid, Lidocaine, Light mineral oil, Liquid Germall Plus (propylene glycol, diazolidinyl urea, iodopropynyl butylcarbamate), Lyophilized formulate porcine plasma, Magnesium aluminum silicate, Magnesium oxide, Magnesium stearate, Magnesium sulfate, Malic acid, Maltodextrin, Manganese chloride, Manganese oxide, Mannitol, Meadowsweet extract, Menthol, Methyl salicylate, Methyl triethoxysilane (MTES), Methylal, Methylene blue, Mineral oil, Molybdenum chloride, Myristyl myristate, Myrtillus extract, Oak extract, Oat glucan, O-cymen-5-ol (Biosol), Olive oil, Ozone, Palm glycerides, Palmitamide MEA, Palmitic acid, Panthenol FCC (form of vitamin B), Parabens (various forms), Paraffin, Pentalyn-H (Pentaerythritol ester of rosin), Pentylene glycol, Petrolatum, Phenoxyethanol, Phosphoric acid, Phosphorus pentoxide, Piroctone olamine, Polyaminopropyl biguanide (PAPB), Polygonum cuspidatum, Polyhexamethylene biguanide (PHMB, polyhexanide), Polymyxin B sulfate, Polyricinoleate, Polyvinyl pyrrolidoneiodine, Potassium ferrate, Potassium iodide, Potassium iron oxyacid salt, Potassium sorbate, Povidone iodine, Povidone USP (Plasdone K 29-32), Propyl gallate, Propylene glycol, Pyroglutamic acid, Quaternium 15, RADA-16 peptide, Rubidium chloride, Saccharin, Salicylic Acid, Sandalwood oil, Sarcosine, Shea butter, Silver (various forms), Silver sulfadiazine, Sodium benzoate, Sodium citrate, Sodium fluoride, Sodium lactate, Sodium metabisulfite, Sodium selenite, Sodium sulfate, Sodium tetraborate (Borax), Solanum lycopersicum (tomato) extract, Sorbic acid, Sorbitan sesquioleate (Arlacel C), Sorbitol, Soy protein, Squalane, Steareth-10, Stearic acid, Styrax, Sucralfate (sucrose octasulfate, aluminum hydrochloride), Sucrose, Sucrose laurate, Sulfur dioxide, Tara Gum, Tartaric acid, Tea tree oil, Telmesteine, Theobroma Grandiflorum seed butter, Thrombin, Thymol, Titanium dioxide, Titanium oxide, Tonalin FFA 80, Transcinnamaldehyde, Triethanolamine (TEA), Triglycerol (polyglycerol-3), Triiodide resin, Trolamine, Tromethamine USP, Vaccinium (blueberry), Vegetable oil, Vitamin C (ascorbic acid), Vitamin E (tocopherol), Vitis vinifera (grape), White petroleum, Wintergreen fragrance, Wood pulp core, Xanthan gum, Xylitol, Zinc (various forms), Zirconium oxide, or combinations thereof.

In some embodiments, a payload that may be included in a biomaterial preparation (e.g., a provided polymer combination preparation) is or comprises a photosensitizer used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer not only has to be in a form suitable for administration, but also in a form that can readily undergo cellular internalization at the target site, preferably with some degree of selectivity over normal tissues.

In some embodiments, a payload that may be included in a biomaterial preparation (e.g., a provided polymer combination preparation) is or comprises a radiosensitizer. A radiosensitizer is typically a molecule, compound or agent that makes target cells more sensitive to radiation therapy. Administration of a biomaterial preparation (e.g., a provided polymer combination preparation) comprising a radiosensitizer to a patient receiving radiation therapy may concentration function of the radiosensitizer on target cells and thereby enhance the effects of radiation therapy.

In some embodiments, a payload that may be included in a biomaterial preparation (e.g., a provided polymer combination preparation) is or comprises a radioisotope. Examples of suitable radioisotopes include any α-, β-, or γ-emitter, which, when localized at a target site, results in cell destruction, including, e.g., but not limited to Examples of such radioisotopes include, but are not limited to, iodine-131, iodine-125, bismuth-212, bismuth-213, astatine-211, rhenium-186, rhenium-188, phosphorus-32, yttrium-90, samarium-153, and lutetium-177.

In some embodiments, a payload that may be included in a biomaterial preparation (e.g., a provided polymer combination preparation) is or comprises a prodrug activating enzyme, e.g., for a directed enzyme prodrug therapy approach. For example, in some embodiments, a biomaterial preparation (e.g., a provided polymer combination preparation) comprising a prodrug activating enzyme and a prodrug can be administered to a subject, wherein the biomaterial preparation forms in situ at a target site and the prodrug activating enzyme included therein converts the prodrug delivered to/around the target site into an active drug. The prodrug can be converted to an active drug in one step (by the prodrug activating enzyme) or in more than one step.

In some embodiments, a payload that may be included in a biomaterial preparation (e.g., a provided polymer combination preparation) is or comprises an anti-angiogenic agent. Anti-angiogenic agents suitable for use in accordance with the present disclosure may include any molecule, compound or factor that blocks, inhibits, slows down or reduce the process of angiogenesis, or the process by which new blood vessels form by developing from pre-existing vessels. Such a molecule, compound or factor can block angiogenesis by blocking, inhibiting, slowing down or reducing any of the steps involved in angiogenesis, including the steps of (1) dissolution of the membrane of the originating vessel, (2) migration and proliferation of the endothelial cells, and (3) formation of new vascular tube by the migrating cells. Examples of anti-angiogenic agents include, but are not limited to, bevacizumab (Avastin®), celecoxib (Celebrex®), endostatin, anti-VEGF antibody, interferon-α, squalamine, cisplatin, combretastatin A-4, and Neovastat.

In some embodiments, a payload that may be included in a biomaterial preparation (e.g., a provided polymer combination preparation) is or comprises an immunomodulatory payload. In some embodiments, an immunomodulatory payload is included in a biomaterial preparation (e.g., a provided polymer combination preparation) as monotherapy. In some embodiments, an immunomodulatory payload is or comprises a modulator of inflammation. As will be understood by appreciated by one of skilled in the art, inflammation may be immunostimulatory or immunosuppressive depending on the biological context. Accordingly, in some embodiments, an immunomodulatory payload is or comprises a modulator of immunostimulatory inflammation. In some embodiments, an immunomodulatory payload is or comprises a modulator of immunosuppressive inflammation. In some embodiments, an immunomodulatory payload is or comprises a modulator of innate immunity and/or adaptive immunity. In some such embodiments, a modulator of innate immunity and/or adaptive immunity is or comprises an agonist of innate immunity and/or adaptive immunity.

In some embodiments, an immunomodulatory payload is or comprises a modulator of granulocytes. Granulocytes are a category of white blood cells in an innate immune system characterized by the presence of granules in their cytoplasm. Granulocytes may also be referred to as polymorphonuclear leukocytes or polymorphonuclear neutrophils (PMN, PML, or PMNL) because of the varying shapes of the nucleus, which is usually lobed into three segments. This distinguishes them from mononuclear agranulocytes. Examples of granulocytes include but are not limited to neutrophils, eosinophils, basophils, and/or mast cells.

In some embodiments, an immunomodulatory payload is or comprises a modulator of agranulocytes. As appreciated by one of skilled in the art, agranulocytes, also known as nongranulocytes or mononuclear leukocytes, are characterized by the absence of granules in their cytoplasm, which distinguishes them from granulocytes. Examples of agranulocytes include but are not limited to lymphocytes, monocytes, and/or macrophages. Lymphocytes, as will be understood by one of skilled in the art, typically include but are not limited to B cells, T cells, natural killer T cells, and/or natural killer (NK) cells.

In some embodiments, an immunomodulatory payload is or comprises a modulator of myeloid cells and/or lymphoid cells. In some embodiments, an immunomodulatory payload is or comprises a modulator of neutrophils, eosinophils, basophils, lymphocytes, and/or monocytes. In some embodiments, an immunomodulatory payload is or comprises a modulator of hematopoietic stem cells, common myeloid progenitors, megakaryocytes, thrombocytes, erythrocytes, mast cells, myeloblasts, basophils, neutrophils, eosinophils, monocytes, macrophages, dendritic cells, common lymphoid progenitors, natural killer cells, T lymphocytes, B lymphocytes, and/or plasma cells.

In some embodiments, an immunomodulatory payload is or comprises an immunomodulatory agent as described in International Patent Publication No. WO 2018/045058 (which includes, e.g., but not limited to examples of activators of innate immune response, activators of adaptive immune response, immunomodulatory cytokines, modulators of macrophage effector functions, etc.) and WO 2019/183216 (which includes, e.g., but not limited to inhibitors of immunosuppressive inflammation, e.g., mediated by a p38 mitogen-activated protein kinase (MAPK) pathway, etc.), the contents of each of which are incorporated herein by reference for purposes described herein. In some embodiments, an immunomodulatory payload is or comprises an activator of innate immune response, for example, in some embodiments, which may be or comprise a stimulator of interferon genes (STING) agonist, a Toll-like receptor (TLR) agonist, and/or an activator of innate immune response as described in International Patent Publication No. WO 2018/045058, the contents of which are incorporated herein by reference for purposes described herein. In some embodiments, an immunomodulatory payload is or comprises an inhibitor of immunosuppressive inflammation, for example, in some embodiments, which may be or comprise a COX2 inhibitor or an inhibitor of immunosuppressive inflammation mediated by a p38 mitogen-activated protein kinase (MAPS) pathway, as described in International Patent Publication No. WO 2019/183216, the contents of which are incorporated herein by reference for purposes described herein.

In some embodiments, an immunomodulatory payload is or comprises a Toll-like receptor 7 and 8 (TLR7/8) agonist (e.g., ones described in the International Patent Publication No. WO 2018/045058). In some embodiments, an exemplary TLR7/8 agonist is or comprises resiquimod (R848) or a variant thereof.

In some embodiments, an immunomodulatory payload is or comprises a COX1 and/or COX2 mediated signaling pathway inhibitor. In some embodiments, an exemplary COX1 and/or COX2 mediated signaling pathway inhibitor is a non-steroidal anti-inflammatory drug (NSAID). In some embodiments, an immunomodulatory payload is or comprises a non-steroidal anti-inflammatory drug (NSAID) (e.g., ones described in the International Patent Publication No. WO 2019/183216). In some embodiments, a NSAID is or comprises ketorolac (including, e.g., ketorolac tromethamine). Ketorolac has been conventionally used for short-term pain management and, therefore, is typically not prescribed for longer than five days owing to toxicity. Systemic exposure of ketorolac can lead to renal and cardiac toxicity as well as bleeding in the gastrointestinal tract. In some embodiments, the present disclosure appreciates that local retention of ketorolac may be desirable. For example, in some embodiments, ketorolac for use in the present disclosure is released from a polymer combination preparation (e.g., as described herein) over a period of at least 2 days or longer, e.g., at least 3 days, at least 4 days, at least 5 days, at least 6 days, at least 7 days, at least 8 days, at least 9 days, at least 10 days, or longer such that the immune system is modulated (e.g., immunosuppressive inflammation induced by tumor resection surgery is inhibited or reduced). Ketorolac may be administered as a racemic mixture or as an individual enantiomer, e.g., the S-enantiomer. In some embodiments, a NSAID comprises lornoxicam. In some embodiments, a NSAID comprises meclofenamate sodium.

In some embodiments, an immunomodulatory payload is or comprises a resolvin (e.g., ones as described in the International Patent Publication No. WO 2019/183216). In some embodiments, an exemplary resolving is or comprises RvD2. RvD2 is a resolvin that acts as a specialized pro-resolving mediator (SPM) involved in a coordinated resolution program that can prevent excessive inflammation and/or resolve acute inflammation.

In some embodiments, an immunomodulatory payload is or comprises a modulator of an adenosine associated pathway (e.g., adenosine metabolism and/or recognition pathway). In certain embodiments, an inhibitor of an adenosine associated pathway may be an inhibitor of A2A and/or A2B receptors. In certain embodiments, inhibitors of A2A and/or A2B may be or comprise Etrumadenant (also known as AB928).

In some embodiments, an immunomodulatory payload is or comprises an inhibitor of Bruton’s tyrosine kinase (BTK). In certain embodiments, an inhibitor of BTK can be or comprises Zanubrutinib (also known as Brukinsa or BGB-3111).

In some embodiments, an immunomodulatory payload is or comprises an inhibitor of CXCR4/CXCL12 mediated signaling. In certain embodiments, an inhibitor of CXCR4/CXCL12 mediated signaling may be but is not limited to Plerixafor.

In some embodiments, an immunomodulatory payload is or comprises a NOD1 and/or NOD2 agonist (e.g., as described in the International Patent Publication No. WO 2018/045058). In some embodiments, an exemplary NOD1 and/or NOD2 agonist may be orcomprises -Ala-γ-D-Glu-mDAP (TriDAP). Tri-DAP is typically present in the peptidoglycan (PGN) of Gram-negative bacilli and certain Gram-positive bacteria. In some embodiments, Tri-DAP is recognized by the intracellular sensor NOD1, which induces a signaling cascade leading to NF-κB activation and/or production of inflammatory cytokines. In some embodiments, an exemplary NOD1 and/or NOD2 agonist may be or comprises MurNAc-L-Ala-γ-D-Glu-mDAP (M-TriDAP). Similar to TriDAP, M-TriDAP is a peptidoglycan (PGN) degradation product found mostly in Gram-negative bacteria. M-TriDAP is typically recognized by the intracellular sensor NOD1 (CARD4) and to a lesser extent NOD2 (CARD15). Recognition of M-TriDAP by NOD 1/NOD2 induces a signaling cascade involving the serine/threonine RIP2 (RICK, CARDIAK) kinase, which interacts with IKK leading to the activation of NF-κB and production of inflammatory cytokines such as TNF-α and IL-6. In some embodiments, M-TriDAP induces the activation of NF-κB at similar levels to Tri-DAP.

In some embodiments, an immunomodulatory payload is or comprises a modulator of an immune cell effector function, survival, and/or recruitment. In some embodiments, an immunomodulatory payload is or comprises a modulator of monocyte effector function, survival, and/or recruitment. In some embodiments, an immunomodulatory payload is or comprises a modulator of a macrophage effector function, survival, and/or recruitment. In some embodiments, an immunomodulatory payload is or comprises a modulator of myeloid derived suppressor cell (MDSC) effector function, survival, and/or recruitment. In some embodiments, an immunomodulatory payload is or comprises a modulator of neutrophil function, survival, and/or recruitment. In some embodiments, an immunomodulatory payload is or comprises a modulator of natural killer cell effector function, survival, and/or recruitment. Examples of such modulators of immune cell effector function, survival, and/or recruitment may include, but are not limited to adenosine A2A receptor (A2AR) inhibitors, chemokines (e.g., CCL1, CCL2, CCL3, CCL4, CCL5, CCL17, CCL19, CCL21, CCL22, CXCL9, CXCL10, CXCL11, CXCL13, CXCL16, and/or CX3CL1, etc.), angiopoietin 2 (ANG2) inhibitors, arginase-1 (ARG1) inhibitors, colony-stimulating factor 1 (CSF1) inhibitors, granulocyte-macrophage-colony-stimulating factor (GM-CSF) inhibitors, colony-stimulating factor 1 receptor (CSF1R) inhibitors, ectonucleoside triphosphate diphosphohydrolase (ENTPD1, also known as CD39) inhibitors, tumor necrosis factor receptor superfamily member 5 (CD40) agonists, OX40 agonists, 4-1BB agonists, CD160 agonists, DNAM agonists, NKG2D agonists, NKG2A inhibitors, TIGIT inhibitors, LILRB1 inhibitors, LILRB2 inhibitors, leukocyte surface antigen CD47 (CD47) inhibitors, signal regulatory protein alpha (SIRPα) inhibitors, 5′-nucleotidase (NT5E, also known as CD73) inhibitors, prostaglandin-endoperoxide synthase 2 (PTGS2, also known as cyclooxygenase-2 (COX-2)) inhibitors, prostaglandin E2 (PGE2) inhibitors, PGE2 receptor 2 (EP2) inhibitors, PGE2 receptor 4 (EP4) inhibitors, inducible nitric oxide synthase (iNOS) inhibitors, fibroblast growth factor 1 (FGF) inhibitors, indoleamine 2,3-dioxygenase (IDO) inhibitors, Class II HDAC (e.g., HDAC4, HDAC5, HDAC6, HDAC7, HDAC9, and HDAC10) inhibitors, Ig-Like Transcript 2 (ILT2) inhibitors, S100A8/A9 inhibitors, RAGE inhibitors, interleukin-8 (IL-8, also known as CXCL8) inhibitors, C-X-C chemokine receptor type 1 (CXCR-1) inhibitors, C-X-C chemokine receptor type 2 (CXCR-2) inhibitors, interleukin-10 (IL-10) inhibitors, interleukin-2 (and variants thereof), interleukin-12 subunit alpha (IL-12a, also known as IL-12) (and variants thereof), interleukin-15 (and variants thereof), interleukin-18 (and variants thereof), Leukotriene B₄ (LTB₄) inhibitors, resolvin family (e.g., RvD1, RvD2, RvD3, RvD4, RvD5, RvD6, 17R-RvD1, 17R-RvD2, 17R-RvD3, 17R-RvD4, 17R-RvD5, 17RRvD6, RvE1, 18S-RvE1, RvE2, RvE3, RvT1, RvT2, RvT3, RvT4, RvDln-3, RvD2n-3, and/or RvD5n-3) specialized pro-resolving mediators (SPMs), lipoxin family (e.g., LxA4, LxB4, 15-epi-LxA4, and/or 15-epiLxB4) SPMs, protectin/neuroprotection (e.g., DHA-derived protectins/neuroprotectins and/or n-3 DPA-derived protectins/neuroprotectins) SPMs, maresins (e.g., DHA-derived maresins and/or n-3DPA-derived maresins) SPMs, phosphoinositide 3 kinase gamma (PI3Kγ) inhibitors, transforming growth factor beta (TGF-β) inhibitors, transforming growth factor beta receptors (TGF-βR family, e.g., ALK1, ALK2, ALK3, ALK4, TGF-βR1, ALK6, ALK7, TGF-βR2, TGF-βR3, BMPR2, ACVR2A, ACVR2B, and/or AMHR2), vascular endothelial growth factor family (VEGF, e.g., VEGF-A, VEGF-B, VEGF-C, and/or VEGF-D) inhibitors, vascular endothelial growth factor receptor family (VEGFR, e.g., VEGFR-1, VEGFR-2, and/or VEGFR-3) inhibitors, JAK/STAT inhibitors, and/or combinations thereof.

Those skilled in the art will appreciate that the human immune system is complex, and rigid classification of a particular agent as one category of immunomodulatory agent (e.g., as an agonist of innate immunity versus of adaptive immunity and/or as a modulator of macrophage effector function, of granulocytes, of myeloid cells and/or lymphoid cells, etc.) is not always useful, necessary, or sometimes even possible. Those skilled in the art, based on descriptions herein, will understand in context the metes and bounds of relevant agents useful in embodiments as described herein. For example, in some embodiments, certain immunomodulatory agents that may be useful as activators of adaptive immune response in one context may be also effective to modulate survival, recruitment, and/or effector function of one or more immune cell types, including, e.g., macrophages, monocytes, myeloid-derived suppressor cells, and/or natural killer cells.

In some embodiments, an immunomodulatory payload is released from a polymer combination preparation and is taken up by immune cells. In some embodiments, immune cells that take up the immunomodulatory payload exhibit at least one of the following biological activities: expressing an immunomodulatory polypeptide in response to an immunomodulatory payload, exhibiting an increased expression of a type 1 interferon in response to innate immune stimulation induced by an immunomodulatory payload, and/or exhibiting a change in level and/or activity of an immunomodulatory polypeptide.

In some embodiments, an immunomodulatory payload is a polynucleotide agent. In some embodiments, a polynucleotide agent is a non-coding polynucleotide that is not translated into a polypeptide. In some embodiments, a non-coding polynucleotide is a dsRNA, an siRNA, an miRNA, an shRNA, or another RNA that initiates an RNA interference reaction. In some embodiments, a polynucleotide is a guide RNA suitable for mediating gene editing. In some embodiments, a polynucleotide agent is a coding polynucleotide that can be translated into a polypeptide. In some embodiments, a biomaterial preparation included in a polymer combination preparation is characterized in that the polynucleotide agent is released from the biomaterial preparation and is taken up by local cells so that at least a subset of local immune cells express the immunomodulatory polypeptide encoded by the polynucleotide agent. In some embodiments, a polymer combination preparation is characterized in that at least a subset of local immune cells have an increased expression of a type 1 interferon in response to innate immune stimulation induced by a polynucleotide agent. In some embodiments, a polymer combination preparation is characterized in that, at least a subset of local immune cells have a change in level and/or activity of an immunomodulatory polypeptide in response to a polynucleotide agent.

In some embodiments, a target cell may comprise myeloid cells and/or plasmacytoid dendritic cells. In some embodiments, a target cell may comprise non-immune cells, such as fibroblasts and/or endothelial cells.

### D. Solvent Systems

In some embodiments, a polymer combination preparation, or individual components of a polymer combination preparation are prepared or present in a suitable solvent system. For example, in some embodiments such a solvent system has a pH ranging from 4.5-8.5. In certain embodiments, a polymer combination preparation, or individual components of a polymer combination preparation is prepared or present in a suitable solvent system having pH 7-9. In certain embodiments, a polymer combination preparation, or individual components of a polymer combination preparation is prepared or present in a suitable solvent system having pH 7-7.5 (e.g., pH 7.4). In certain embodiments, a polymer combination preparation, or individual components of a polymer combination preparation is prepared or present in a suitable solvent system having pH 7.5-8.5. In certain embodiments, a polymer combination preparation, or individual components of a polymer combination preparation is prepared or present in a suitable solvent system having pH 8.

In certain embodiments, a polymer combination preparation, or individual components of such a polymer combination preparation are prepared or present in water. In some embodiments, a polymer combination preparation, or individual components of such a polymer combination preparation are prepared or present in an aqueous buffer system. In some embodiments, such an aqueous buffer system may comprise one or more salts (e.g., but not limited to sodium phosphate, and/or sodium hydrogen carbonate). In some embodiments, such a solvent system is an aqueous buffer system having a higher buffering capacity than a 10 mM phosphate buffer. In some embodiments, such a solvent system is an aqueous buffer system having a higher buffering capacity than a 20 mM phosphate buffer. In certain embodiments, a polymer combination preparation, or individual components of such a polymer combination preparation are prepared or present in a phosphate buffer, e.g., phosphate-buffered-saline (PBS). In certain embodiments, a polymer combination preparation, or individual components of such a polymer combination preparation are prepared or present in a bicarbonate buffer. In some embodiments, polymer combination preparations, and/or individual components thereof are prepared or present in an aqueous buffer system having a concentration range of from 1 mM to 500 mM, or from 5 mM to 250 mM, or from 10 mM to 150 mM, or from 1 mM to 50 mM, or from 5 mM to 50 mM or from 5 mM to 100 mM, or from 50 mM to 100 mM. In certain embodiments, a suitable aqueous buffer (e.g., a phosphate buffer) is prepared at a concentration of 10 mM to 50 mM. In certain embodiments, a suitable aqueous buffer (e.g., a phosphate buffer) is prepared at a concentration of 10 mM to 30 mM. In certain embodiments, a suitable aqueous buffer (e.g., a bicarbonate buffer) is prepared at a concentration of 100 mM to 200 mM. In certain embodiments, a polymer combination preparation, or individual components thereof are prepared or present in a sodium phosphate buffer at a concentration of 10 mM to 50 mM or 10 mM or 30 mM. In some embodiments, an aqueous buffer system may comprise 0.9% saline.

### E. Optional Additives

In some embodiments, a polymer combination preparation may comprise one or more additives. In some embodiments, such an additive may be or comprise a thickening agent. As will be understood by one of skilled in the art, such a thickening agent may improve suspensions of components or emulsions, which increase stability of a combination. In some embodiments, such a thickening agent may be useful to prevent, reduce, or delay phase separation of individual polymer components in a polymer combination preparation. Examples of thickening agents may include, but are not limited to cellulose derivatives, starches, pectin, xanthan, and/or any combinations thereof.

### II. Certain Properties And/or Characteristics of Provided Polymer Combination Preparations or Compositions Comprising the Same

Provided polymer combination preparations or compositions comprising the same may be characterized by one or more (e.g., one, two, three, or more) of certain properties and/or characteristics described herein. Those skilled in the art, reading the present disclosure, will appreciate that provided polymer combination preparations or compositions comprising the same may be configured to provide suitable material properties and/or characteristics for a particular application. For example, in some embodiments, suitable material properties and/or characteristics for a particular application may be determined, for example based on characteristics of tissue surrounding a tumor, administration routes, administration sites, and/or desired duration of immunomodulation in which a method is being practiced.

### A. Immunomodulatory Characteristics

In some embodiments, a provided polymer combination preparation may be non-immunomodulatory. In some such embodiments, a provided polymer combination preparation and/or a composition comprising the same may comprise an immunomodulatory payload (e.g., as described herein) such that the resulting composition or preparation is immunomodulatory.

In some embodiments, a provided polymer combination preparation comprising poloxamer may comprise a second polymer component or an additional polymer component such that the resulting polymer combination preparation itself may be immunomodulatory in the absence of an immunomodulatory payload. For example, in some embodiments, such a resulting polymer combination preparation itself may be useful for inducing innate immunity agonism. In some embodiments, such a resulting polymer combination preparation itself may be useful for resolving or reducing inflammation, which in some embodiments may be or comprise immunosuppressive inflammation. In some embodiments, not only is such a polymer combination preparation substantially free of an immunomodulatory payload, but also a composition or preparation comprising such a polymer combination preparation of the present disclosure may not necessarily require inclusion of at least one or more (e.g., at least two or more, at least three or more) types of immunomodulatory payloads, including, e.g., innate immunity immunomodulatory payloads, adaptive immunity modulatory payloads, immunomodulatory cytokines, immunomodulatory chemotherapeutics, immunomodulatory therapeutic agents, and/or combinations thereof. In some embodiments, an immunomodulatory composition of the present disclosure comprises a provided polymer combination preparation in the absence of an immunomodulatory payload.

In some embodiments, a provided polymer combination preparation and/or a composition or preparation comprising a provided polymer combination preparation can, indirectly or directly, activate one or more pattern recognition receptors of one or more types of cells of an innate immune system, such as, e.g., dendritic cells, macrophages, monocytes, neutrophils, and/or natural killer (NK) cells, such that at least one or more innate immune responses are induced (e.g., as described herein). Examples of such a pattern recognition receptor is or comprises a C-type Lectin Receptor (CLR), a Nucleotide-binding Oligomerization Domain-Like Receptor (NOD-Like receptor or NLR), a Retinoic acid-inducible gene-I-Like Receptor (RLR), and/or a Toll-Like Receptor (TLR). In some embodiments, a provided polymer combination preparation and/or a composition or preparation comprising a provided polymer combination preparation can, directly or indirectly, activate at least one or more C-type Lectin Receptors (CLRs) of many different cells of an innate immune system (e.g., dendritic cells, macrophages, etc.), which include, e.g., mannose receptors, and/or asialoglycoprotein receptor family (e.g., Dectin-1, Dectin-2, macrophage-inducible C-type lectin (Mincle), dendritic cell-specific ICAM3-grabbing nonintegrin (DC-SIGN), and DC NK lectin group receptor-1 (DNGR-1)). In some embodiments, a provided polymer combination preparation and/or a composition or preparation comprising a provided polymer combination preparation can, directly or indirectly, activate at least one or more NOD-Like Receptors (NLRs) of different types of leukocytes (e.g., lymphocytes, macrophages, dendritic cells), which include, e.g., NLRA (e.g., CIITA), NLRB (e.g., NAIP), NLRC (e.g., NOD1, NOD2, NLRC3, NLRC4, NLRC5, NLRX1) and/or NLRP (e.g., NLRP1, NLRP2, NLRP3, NLRP4, NLRP5, NLRP6, NLRP7, NLRP8, NLRP9, NLRP10, NLRP11, NLRP12, NLRP13, NLRP14). In some embodiments, a provided polymer combination preparation and/or a composition or preparation comprising a provided polymer combination preparation can, directly or indirectly, activate at least one or more RIG-I-Like Receptors (RLRs) of, e.g., myeloid cells, which include, e.g., RIG-I, MDA5, and/or LGP2. In some embodiments, a provided polymer combination preparation and/or a composition or preparation comprising a provided polymer combination preparation can, directly or indirectly, activate at least one or more Toll-Like Receptors (TLRs) of different types of leukocytes (e.g., dendritic cells, myeloid dendritic cells, monocytes, macrophages, and/or neutrophils), which include, e.g., TLR1, TLR2, TLR3, TLR4, TLR5, TLR6, TLR7, TLR8, TLR9, and/or TLR10.

In some embodiments, a provided polymer combination preparation and/or a composition or preparation comprising a provided polymer combination preparation can, indirectly or directly, activate or induce (e.g., increase level and/or activity of) an inflammasome, e.g., in myeloid cells, such that at least one or more innate immune responses (and/or one or more features of an innate immune response) are induced (e.g., as described herein). In some embodiments, an inflammasome is typically a multi-protein complex that activates one or more inflammatory responses, such as, e.g., promoting maturation and/or secretion of one or more proinflammatory cytokines such as, e.g., interleukin 1β and/or interleukin 18. In some embodiments, a provided polymer combination preparation and/or a composition or preparation comprising a provided polymer combination preparation can, indirectly or directly, activate or induce (e.g., increase level and/or activity of) an inflammasome comprising an Absent in Melanoma 2 (AIM2)-Like Receptor (“AIM2 inflammasome”). In some embodiments, a provided polymer combination preparation and/or a composition or preparation comprising a provided polymer combination preparation can, indirectly or directly, activate or induce (e.g., increase level and/or activity of) an inflammasome comprising one or more NLRs, including, e.g., NLRP1 (e.g., NALP1b), NLRP3 (e.g., NALP3), and/or NLRC4 (e.g., IPAF).

In some embodiments, a provided polymer combination preparation and/or a composition or preparation comprising a provided polymer combination preparation can, directly or indirectly, activate one or more components involved in a cGAS-STING pathway (e.g., a cGAS-STING pathway and/or components thereof as described in Chen et al., “Regulation and function of the cGAS-STING pathway of cytosolic DNA sensing” Nature Immunology (2016) 17: 1142-1149); which is incorporated herein by reference in its entirety for the purpose described herein, such that innate immunity is induced. In some embodiments, a provided polymer combination preparation and/or a composition or preparation comprising a provided polymer combination preparation can, directly or indirectly, induce activity and/or level of NFκB and/or other components associated with an NFκB pathway (e.g., NFκB activation during innate immune response, e.g., as described in Dev et al., “NF-κB and innate immunity” Curr. Top. Microbiol. Immunol.(2011) 349: 115-43); which is incorporated herein by reference in its entirety for the purpose described herein. In some embodiments, a provided polymer combination preparation and/or a composition or preparation comprising a provided polymer combination preparation can, directly or indirectly, lead to production of reactive oxygen species, e.g., during innate immune response.

As will be clear to one of those skilled in the art reading the present disclosure, in some embodiments, a provided polymer combination preparation and/or a composition or preparation comprising a provided polymer combination preparation can, directly or indirectly, activate one or more of components and/or pathways (e.g., ones as described herein) associated with activation of innate immunity. For example, in some embodiments, a provided polymer combination preparation and/or a composition or preparation comprising a provided polymer combination preparation can, directly or indirectly, activate one or more pattern recognition receptors of one or more types of cells of an innate immune system (e.g., ones as described herein) and also activate or induce (e.g., increase level and/or activity of) an inflammasome, e.g., in myeloid cells.

### B. Viscosity

In some embodiments, a polymer combination preparation described herein (e.g., a precursor state or a polymer network state such as a viscous solution) can be characterized by a viscosity of no more than 25,000 mPa·s or lower, including, e.g., no more than 24,000 mPa·s, no more than 23,000 mPa·s, no more than 22,000 mPa·s, no more than 21,000 mPa·s, no more than 20,000 mPa·s, no more than 19,000 mPa·s, no more than 18,000 mPa·s, no more than 17,000 mPa·s, no more than 16,000 mPa·s, no more than 15,000 mPa·s, no more than 14,000 mPa·s, no more than 13,000 mPa·s, no more than 12,000 mPa·s, no more than 11,000 mPa·s, no more than 10,000 mPa·s, no more than 9,000 mPa·s, no more than 8,000 mPa·s, no more than 7,000 mPa·s, no more than 6,000 mPa·s, no more than 5,000 mPa·s, no more than 4,000 mPa·s, no more than 3,500 mPa·s, no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, a polymer combination preparation described herein (e.g., a precursor state or a polymer network state such as, e.g., a viscous solution) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, at least 3,000 mPa·s, at least 4,000 mPa·s, at least 5,000 mPa·s, at least 6,000 mPa·s, at least 7,000 mPa·s, at least 8,000 mPa·s, at least 9,000 mPa·s, at least 10,000 mPa·s, at least 11,000 mPa·s, at least 12,000 mPa·s, at least 13,000 mPa·s, at least 14,000 mPa·s, at least 15,000 mPa·s, at least 16,000 mPa·s, at least 17,000 mPa·s, at least 18,000 mPa·s, at least 19,000 mPa·s, at least 20,000 mPa·s, at least 21,000 mPa·s, at least 22,000 mPa·s, at least 23,000 mPa·s, at least 24,000 mPa·s, or higher. Combinations of the above-mentioned ranges are also possible. For example, in some embodiments, a polymer combination preparation described herein (e.g., a precursor state or a polymer network state such as, e.g., a viscous solution) may be characterized by a viscosity of 5 mPa·s to 10,000 mPa·s, or 10 mPa·s to 5,000 mPa·s, or 5 mPa·s to 200 mPa·s, or 20 mPa·s to 100 mPa·s, or 5 mPa·s to 20 mPa·s, or 3 mPa·s to 15 mPa·s. In some embodiments, a polymer combination preparation described herein (e.g., a precursor state or a polymer network state such as, e.g., a viscous solution) can be a viscous solution with a viscosity similar to honey (e.g., with mPa·s and/or centipoise similar to honey, e.g., approximately 2,000 to 10,000 mPa·s). In some embodiments, a polymer combination preparation described herein (e.g., a precursor state or a polymer network state such as, e.g., a viscous solution) can be a viscous solution with a viscosity similar to natural syrup (e.g., a syrup from tree sap, a syrup from molasses, etc.) (e.g., with mPa·s and/or centipoise similar to natural syrups, e.g., approximately 15,000 to 20,000 mPa·s). In some embodiments, a polymer combination preparation described herein (e.g., a precursor state or a polymer network state such as, e.g., a viscous solution) can be a viscous solution with a viscosity similar to ketchup (e.g., tomato ketchup, e.g., with mPa·s and/or centipoise similar to ketchup, e.g., approximately 5,000 to 20,000 mPa·s). One skilled in the art reading the present disclosure will appreciate that, in some cases, viscosity of a polymer combination preparation described herein may be selected or adjusted based on, e.g., administration routes (e.g., injection vs. implantation), injection volume and/or time, and/or impact duration of innate immunity stimulation. As will be also understood by one skilled in the art, viscosity of a polymer depends on, e.g., temperature and concentration of the polymer in a testing sample. In some embodiments, viscosity a polymer combination preparation described herein may be measured at 20° C., e.g., with a shear rate of 1000 s⁻¹.

In some embodiments, a polymer combination preparation (e.g., a precursor state or a polymer network state such as, e.g., a viscous solution) comprising poloxamer (e.g., as described herein) may be characterized by a viscosity of no more than 3,500 mPa·s or lower, including, e.g., no more than 3,000 mPa·s, no more than 2,500 mPa·s, no more than 2,000 mPa·s, no more than 1,500 mPa·s, no more than 1,000 mPa·s, no more than 500 mPa·s, no more than 250 mPa·s, no more than 200 mPa·s, no more than 150 mPa·s, no more than 100 mPa·s, no more than 75 mPa·s, no more than 50 mPa·s, no more than 25 mPa·s, no more than 20 mPa·s, no more than 15 mPa·s, no more than 10 mPa·s, or lower. In some embodiments, polymer combination preparations (e.g., a precursor state or a polymer network state such as, e.g., a viscous solution) comprising poloxamer (e.g., as described herein) may be characterized by a viscosity of at least 5 mPa·s or higher, including, e.g., at least 10 mPa·s, at least 20 mPa·s, at least 30 mPa·s, at least 40 mPa·s, at least 50 mPa·s, at least 60 mPa·s, at least 70 mPa·s, at least 80 mPa·s, at least 90 mPa·s, at least 100 mPa·s, at least 125 mPa·s, at least 150 mPa·s, at least 175 mPa·s, at least 250 mPa·s, at least 500 mPa·s, at least 1,000 mPa·s, at least 1,500 mPa·s, at least 2,000 mPa·s, at least 2,500 mPa·s, or higher. Combinations of the above-mentioned ranges are also possible. For example, in some embodiments, such a viscous polymer solution (e.g., a precursor state or a polymer network state such as, e.g., a viscous solution) may be characterized by a viscosity of 5 mPa·s to 3,000 mPa·s, or 5 mPa·s to 300 mPa·s, 5 mPa·s to 200 mPa·s, or 20 mPa·s to 200 mPa·s, or 5 mPa·s to 20 mPa·s. In some embodiments, viscosity of a polymer combination preparation described herein may be measured at 20° C., e.g., with a shear rate of 1000 s⁻¹.

Among other things, the present disclosure appreciates that hydrogel technologies comprising certain crosslinking technologies (e.g., certain chemical crosslinking technologies, ultraviolet light, etc.) may produce toxic by-products and/or may adversely affect stability or efficacy of agents (e.g., therapeutic agents) that may be combined with a polymer combination preparation.

Alternatively or additionally, the present disclosure appreciates that, in some embodiments, particular advantages can be achieved by administering component(s) of a polymer combination preparation so that an immunomodulatory composition as described herein is formed during and/or upon administration as compared with pre-forming (e.g., by crosslinking) a polymer biomaterial prior to introducing it into a subject. For example, administration of a preformed biomaterial requires proportionate incisions and/or surgical interventions to facilitate administration. In some embodiments, for example, the present disclosure appreciates that such pre-forming generates a material with a defined size and/or structure, which may restrict options for administration, as the dimensions of the pre-formed material may differ from those of a target site (e.g., a resection cavity). In some embodiments a hydrogel may be formed during and/or upon administration. In some embodiments, a polymer combination preparation administered to a target site may comprise a pre-formed hydrogel polymer combination preparation.

In some embodiments, the present disclosure appreciates that a polymer combination preparation that is useful for administration to a target site described herein may be a viscous liquid solution. For example, in some embodiments, a liquid polymer combination preparation may be introduced to a target site so that an immunomodulatory composition as described herein in a form of a viscous solution (e.g., a solution with a viscosity of about 5,000 to 15,000 centipoise at body temperature, e.g., a solution with a viscosity of about 10,000 centipoise at body temperature) is formed upon administration to a target site.

In some embodiments, the present disclosure appreciates that a polymer combination preparation that is useful for administration to a target site described herein may be a viscous liquid solution, which can be substantially retained at the target site upon administration for a certain period of time. In some embodiments, such a viscous liquid polymer combination preparation has a viscosity that is low enough to be injectable (e.g., through a syringe tip or a catheter and/or a syringe needle) but is high enough to be substantially retained at a target site upon administration for a certain period of time. In some embodiments, such a viscous liquid polymer combination preparation may have a viscosity of about 500 to 10,000 centipoise at room temperature. In some embodiments, such a viscous liquid polymer combination preparation may have a viscosity of about 500 to 3,000 centipoise at room temperature. In some embodiments, such a viscous liquid polymer combination preparation may have a viscosity of about 1,000 to 8,000 centipoise at room temperature. In some embodiments, such a viscous liquid polymer combination preparation may have a viscosity of about 2,000 to 6,000 centipoise at room temperature. In some embodiments, such a viscous liquid polymer combination preparation may have a viscosity of about 3,000 to 7,000 centipoise at room temperature. In some embodiments, such a viscous liquid polymer combination preparation may have a viscosity of about 4,000 to 8,000 centipoise at room temperature. In some embodiments, such a viscous liquid polymer combination preparation may have a viscosity of about 5,000 to 9,000 centipoise at room temperature. In some embodiments, such a viscous liquid polymer combination preparation may have a viscosity of about 6,000 to 10,000 centipoise at room temperature.

In some embodiments, the present disclosure appreciates that there may be a viscosity constraint and/or limit on injectability of a liquid polymer combination preparation. For example, in some embodiments, an injectable polymer combination preparation may be characterized by a viscosity amenable to loading and controlled release through a needle of a set gauge (e.g., a needle with a gauge of between 14 and 20, e.g., a needle with a gauge of 16-18). Alternatively, in some embodiments, an injectable polymer combination preparation may be characterized by a viscosity amenable to loading and controlled release through a syringe tip of a set diameter (i.e., without a connected needle, or with a catheter). In some embodiments, a polymer combination preparation included in an immunomodulatory composition (e.g., as described herein) loaded into a syringe may further comprise a plasticizer.

The present disclosure provides technologies, including particular polymer combination preparations, and methods of administration, that permit interventions that may be less invasive than implantation and/or less toxic than systemic administration. In some such embodiments, preparations with improved administration characteristics may be administered in a liquid state; in some embodiments they may be administered in a pre-formed gel state characterized by flexible space-filling properties; in some embodiments they may be administered subcutaneously; in some embodiments they may function as a proximal depot for sustained release of immunomodulatory payloads (e.g., ones described herein); in some embodiments they may permit reprogramming of tissues (e.g., such as tumors and/or e.g., such as sentinel and/or draining lymph nodes); in some embodiments they may be administered prior to or contemporaneously with a tumor resection surgery; in some embodiments, they may be administered ipsilaterally when compared to a tumor resection site and/or primary tumor site; in some embodiments, they may be administered contralaterally when compared to a tumor resection site and/or primary tumor site; in some embodiments, they may be administered to patients who have metastatic, disseminated, and/or recurrent cancers. In some such embodiments, provided preparations are comprised of a relevant material in particulate form (e.g., so that the preparations comprise a plurality of particles, e.g., characterized by a size distribution and/or other parameters as described herein).

### C. Storage Modulus: Polymer Network State

In some embodiments when a polymer combination preparation described herein is in a polymer network state, such a polymer network state may be characterized by a storage modulus of at least 100 Pa, at least 200 Pa, at least 300 Pa, at least 400 Pa, at least 500 Pa, at least 600 Pa, at least 700 Pa, at least 800 Pa, at least 900 Pa, at least 1,000 Pa, at least 1,100 Pa, at least 1,200 Pa, at least 1,300 Pa, at least 1,400 Pa, at least 1,500 Pa, at least 1,600 Pa, at least 1,700 Pa, at least 1,800 Pa, at least 1,900 Pa, at least 2,000 Pa, at least 2,100 Pa, at least 2,200 Pa, at least 2,300 Pa, at least 2,400 Pa, at least 2,500 Pa, at least 2,600 Pa, at least 2,700 Pa, at least 2,800 Pa, at least 2,900 Pa, at least 3,000 Pa, at least 3,500 Pa, at least 4,000 Pa, at least 4,500 Pa, at least 5,000 Pa, at least 6,000 Pa, at least 7,000 Pa, at least 8,000 Pa, at least 9,000 Pa, at least 10,000 Pa, at least 11,000 Pa, at least 12,000 Pa, at least 13,000 Pa, at least 14,000 Pa, at least 15,000 Pa, or higher. In some embodiments, such a polymer network state of a provided polymer combination preparation may be characterized by a storage modulus of no more than 15 kPa, no more than 14 kPa, no more than 13 kPa, no more than 12 kPa, no more than 11 kPa, no more than 10 kPa, no more than 9 kPa, no more than 8 kPa, no more than 7 kPa, no more than 6 kPa, or lower. Combinations of the above-mentioned ranges are also possible. For example, in some embodiments, such a polymer network state of a provided polymer combination preparation may be characterized by a storage modulus of 100 Pa to 15 kPa, or 100 Pa to 10 kPa, or 100 Pa to 7.5 kPa, or 200 Pa to 5,000 Pa, or 300 Pa to 2,500 Pa, or 500 Pa to 2,500 Pa, or 100 Pa to 500 Pa. In some embodiments, a polymer network state of a provided polymer combination preparation may be characterized by a storage modulus of 1,000 Pa to 10,000 Pa, or 2,000 Pa to 10,000 Pa, or 3,000 Pa to 10,000 Pa, or 4,000 Pa to 10,000 Pa, or 5,000 Pa to 10,000, or 6,000 Pa to 10,000 Pa. One of those skilled in the art will appreciate that various rheological characterization methods (e.g., as described in Weng et al., “Rheological Characterization of in situ Crosslinkable Hydrogels Formulated from Oxidized Dextran and N-Carboxyethyl Chitosan” Biomacromolecules, 8: 1109-1115 (2007)) can be used to measure storage modulus of a material, and that, in some cases, storage modulus of a material may be measured with a rheometer and/or dynamic mechanical analysis (DMA). One of those skilled in the art will also appreciate that rheological characterization can vary with surrounding condition, e.g., temperature and/or pH. Accordingly, in some embodiments, a provided polymer combination preparation is characterized by a storage modulus (e.g., as described herein) measured at a body temperature of a subject (e.g., 37° C. of a human subject), e.g., at a pH 5-8 or at a physiological pH (e.g., pH 7). As will be clear to one skilled in the art reading the disclosure provided herein, a storage modulus of a provided polymer combination preparation, e.g., in a form of particles, refers to a bulk storage modulus of particles in a population.

In some embodiments, a polymer network state of a polymer combination preparation provided herein may be characterized by a storage modulus lower than that of an 18 wt% poloxamer hydrogel. For example, in some embodiments, a polymer network state of a polymer combination preparation provided herein may be characterized by a storage modulus, as measured at 37° C., that is reduced by at least 10% or more, including, e.g., at least 20%, at least 30%, at least 40%, at least 50%, at least 60%, or more, as compared to that of an 18% (w/w) poloxamer hydrogel.

In some embodiments, a polymer network state of a polymer combination preparation provided herein may be characterized by a storage modulus (e.g., as described herein) that maintains substantially the same (e.g., within 20% or within 10% or within 5%) when stored at an appropriate temperature for a period of time. For example, in some embodiments, a polymer network state of a polymer combination preparation provided herein may be characterized by a storage modulus (e.g., as described herein), as measured at 37° C., that maintains substantially the same (e.g., within 20% or within 10% or within 5%) when stored at a temperature of 4° C. - 10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer network state of a polymer combination preparation provided herein may be characterized by a storage modulus (e.g., as described herein), as measured at 37° C., that maintains substantially the same (e.g., within 20% or within 10% or within 5%) when stored at a room temperature (e.g., 20° C.-25° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

### D. Phase Angle: Polymer Network State

In some embodiments, a polymer network state of a provided polymer combination preparation can be characterized by a phase angle indicative of a viscoelastic material. For example, in some embodiments, a polymer network state of a provided polymer combination preparation can be characterized by a phase angle of 1° to 50°, or 2° to 45°, or 3° to 40°, or 3° to 35°, 3° to 30°, or 3° to 25°, or 5° to 30°, or 10° to 30°, 15° to 25°, 20° to 35°. In some embodiments, a polymer network state of a provided polymer combination preparation can be characterized by a phase angle of 10° to 30° or 15° to 25°. In some embodiments, a polymer network state of a provided polymer combination preparation can be characterized by a phase angle of 5° to 15° or 10° to 20°. As will be understood by one skilled in the art, phase angle of a polymer biomaterial may be determined by dynamical mechanical analysis, e.g., a frequency sweep analysis, which include, e.g., determination of shear storage modulus and shear loss modulus of a sample. One skilled in the art will appreciate that a storage or elastic modulus of a material may be determined based on its stored energy and it represents the elastic property of the material, while a loss or viscous modulus may be determined based on the energy dissipated as heat and it represents the viscous property of the material. The phase angle (delta) is the arctangent of the ratio of a storage modulus to a loss modulus and its value indicates if the material is more elastic or viscous. Typically, a phase angle of > 45° indicates that the viscous property dominates and the material behaves more like a solution. As the phase angle approaches 0°, the elastic (solid or gel-like) property dominates. For example, a material with a high storage modulus and a low phase angle indicates a stronger gel (more elastic) than one with a lower storage modulus and phase angle. In some embodiments, the phase angle of a provided polymer combination preparation (e.g., as described herein) in a polymer network state may be determined from a frequency sweep analysis performed at a temperature corresponding to the body of the body temperature of a subject to be treated. In some embodiments, a frequency sweep analysis may be performed over a frequency range of 0.1 to 10 Hz with application of a constant 0.4% strain.

### E. Dissolution/Degradation Rate

Polymer combination preparations described herein are typically biocompatible. In some embodiments, at least one polymer component in provided polymer combination preparations may be biodegradable in vivo. In some embodiments, at least one polymer component in provided polymer combination preparations may be resistant to biodegradation (e.g., via enzymatic and/or oxidative mechanisms). In some embodiments, at least one polymer component in provided polymer combination preparations may be chemically oxidized. Accordingly, in some embodiments, polymer combination preparations are able to be degraded, chemically and/or biologically, within a physiological environment, such as within a subject’s body, e.g., at a target site of a subject. One of those skilled in the art will appreciate, reading the present disclosure, that degradation rates of provided polymer combination preparations may vary, e.g., based on selection of a poloxamer type and/or a second polymer (e.g., a carbohydrate polymer such as hyaluronic acid and/or chitosan as described herein in some embodiments) and their material properties, and/or concentrations thereof (e.g., as described herein). For example, the half-life of provided polymer combination preparations (the time at which 50% of a polymer combination preparation is degraded into monomers and/or other non-polymeric moieties) may be on the order of days, weeks, months, or years. In some embodiments, polymer combination preparations described herein may be biologically degraded, e.g., by enzymatic activity or cellular machinery, for example, through exposure to a lysozyme (e.g., having relatively low pH), or by simple hydrolysis. In some cases, provided polymer combination preparations may be broken down into monomers (e.g., polymer monomers) and/or non-polymeric moieties that are non-toxic to cells. As will be understood by one of those skilled in the art, a provided polymer combination preparation has a longer residence time at a target site (e.g., a tumor resection site) upon administration if such a provided polymer combination preparation has a slower in vivo degradation rate.

In some embodiments, a polymer combination preparation provided herein remains substantially homogenous (e.g., no detectable phase separation) when stored at a temperature of 4° C.-10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer combination preparation provided herein remains substantially homogenous (e.g., no detectable phase separation) when stored at a room temperature for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

In some embodiments, a polymer combination preparation provided herein may be characterized in that no more than 20% or less, including, e.g., no more than 15%, no more than 10%, no more than 8%, no more than 6%, no more than 5%, no more than 4%, no more than 3%, no more than 2%, no more than 1%, or less, of the polymer combination preparation is degraded (e.g., via biodegradation or chemical degradation) when stored at a temperature of 4° C.-10° C. (e.g., 4° C., 5° C., 6° C., 7° C., 8° C., 9° C., or 10° C.) for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer. In some embodiments, a polymer combination preparation provided herein may be characterized in that no more than 20% or less, including, e.g., no more than 15%, no more than 10%, no more than 8%, no more than 6%, no more than 5%, no more than 4%, no more than 3%, no more than 2%, no more than 1%, or less, of the polymer combination preparation is degraded (e.g., via biodegradation or chemical degradation) when stored at a room temperature for a period of time, e.g., at least 1 week or longer, including, e.g., at least 2 weeks, at least 3 weeks, at least 4 weeks, at least 5 weeks, at least 6 weeks, at least 7 weeks, at least 2 months, at least 3 months, at least 4 months, at least 5 months, at least 6 months, or longer.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 2 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 2 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 2 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 3 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 3 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 3 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 5 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 5 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 5 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 7 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 7 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 7 days or more after the administration.

In some embodiments, a provided polymer combination preparation is characterized in that, when assessed in vivo by administering to a target site (e.g., a tumor resection site) in a test subject (e.g., as described herein), at least 10% or more, including, e.g., at least 20%, at least 30%, at least 40%, at least 50%, at least 60%, at least 70%, at least 80%, at least 90%, or more, of such a provided polymer combination preparation remains at the target site in vivo 14 days or more after the administration. In some embodiments, less than or equal to 90%, less than or equal to 80%, less than or equal to 70%, less than or equal to 60%, less than or equal to 50%, less than or equal to 40%, less than or equal to 30%, less than or equal to 20%, or lower, of such a provided polymer combination preparation remains at a target site in vivo 14 days or more after the administration. Combinations of the above-mentioned are also possible. For example, in some embodiments, a provided polymer combination preparation is characterized in that, when assessed in vivo by administering to a target site (e.g., a tumor resection site) in a test subject (e.g., as described herein), 30%-80% or 40%-70% of such a provided polymer combination preparation remains at the target site in vivo 14 days or more after the administration.

In some embodiments, a provided polymer combination preparation is characterized in that, when assessed in vivo by administering to a target site (e.g., a tumor resection site) in a test subject (e.g., as described herein), no more than 10% or less, including, e.g., no more than 9%, no more than 8%, no more than 7%, no more than 6%, no more than 5%, no more than 4%, no more than 3%, no more than 2%, no more than 1% or less, of such a provided polymer combination preparation remains at the target site in vivo 10 days or more after the administration.

In some embodiments where a provided polymer combination preparation is immunomodulatory (e.g., acting as a polymeric biomaterial agonist of innate immunity as described in PCT/US20/31169 filed May 1, 2020 (published as WO2020/223698A1)), a provided polymer combination preparation is characterized in that, when assessed in vivo by administering to a target site (e.g., a tumor resection site) in a test subject (e.g., as described herein), such a provided polymer combination preparation is dissolved or degraded at a rate such that an immune response is modulated in one or more aspects. For example, in some embodiments, such a provided polymer combination preparation is dissolved or degraded at a rate such that innate immunity is stimulated in one or more aspects (e.g., activation of a pattern recognition receptor, an inflammasome, and/or a cGAS-STING pathway; and/or production of proinflammatory cytokines and/or upregulation of antigen presentation machinery and/or costimulatory molecules) for a period of at least 2 days or more, including, e.g., at least 3 days, at least 4 days, at least 5 days, at least 6 days, at least 7 days, at least 9 days, at least 10 days or more. In some embodiments, such a provided polymer combination preparation is dissolved or degraded at a rate such that innate immunity is stimulated in one or more aspects (e.g., ones as described herein, including, e.g., but not limited to activation of a pattern recognition receptor, an inflammasome, and/or a cGAS-STING pathway; and/or production of proinflammatory cytokines and/or upregulation of antigen presentation machinery and/or costimulatory molecules) for a period of no more than 15 days or fewer, including, e.g., no more than 10 days, no more than 9 days, no more than 8 days, no more than 7 days, no more than 6 days, no more than 5 days, no more than 4 days, no more than 3 days or fewer.

### F. Payload Release Rate

In some embodiments, polymer combination preparations described herein may be useful to deliver one or more payloads. For example, in some embodiments, one or more payloads may be distributed in a polymer combination preparation such that when administered at a target site (e.g., at a tumor resection site), the polymer combination preparation extends the release of the therapeutic agent at the target site relative to administration of the same therapeutic agent in solution. In certain embodiments, such a polymer combination preparation can extend the release of the therapeutic agent at a target site (e.g., at a tumor resection site) relative to administration of the same therapeutic agent in solution by at least 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 11 hours, 12 hours, 18 hours, 24 hours, 2 days, 3 days, 4 days, 5 days, 6 days, 7 days, 2 weeks, 3 weeks, or 4 weeks. In some embodiments, such a polymer combination preparation can extend the release of a therapeutic agent so that, when assessed at a specified time point after administration, more therapeutic agent is present at a target administration site (e.g., a tumor resection site) than that is observed when the therapeutic agent is administered in solution. For example, in some embodiments, when assessed at 24 hours after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 48 hours after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 3 days after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution. In some embodiments, when assessed at 5 days after administration, the amount of therapeutic agent released to and present at a target administration site (e.g., a tumor resection site) is at least 30% more (including, e.g., at least 40%, at least 50%, at least 60%, at least 70%, at least 80%, at least 90%, or more) than that is observed when the therapeutic agent is administered in solution.

Without wishing to be bound by theory, in some embodiments, solubility property and/or functional group(s) of a payload may impact rate of such a payload released from a polymer network state of a polymer combination preparation described herein. For example, in some embodiments, a hydrophilic agent may be more soluble in an aqueous environment than a lipophilic agent, which may, among other things, contribute to a faster release rate of a hydrophilic agent observed in a provided polymer combination preparation. In some embodiments, a functional group of a payload may interact with a functional group of a polymer in a polymer combination preparation described herein, which may, among other things, contribute to a longer residence time of such a payload in the polymer combination preparation.

In some embodiments, a polymer network state of a provided polymer combination preparation is characterized in that, when tested in vitro at 37° C., it releases a lipophilic agent incorporated therein at a comparable rate as with an 18% (w/w) poloxamer hydrogel (e.g., 18% (w/w) P407 hydrogel). For example, in some embodiments, the 48-hour release profile (e.g., percent release over a period of 48 hours) of an incorporated lipophilic agent for a provided polymer combination preparation is comparable (e.g., within 20%, or within 15%, or within 10%, or within 5%) to that of the same lipophilic agent for an 18% (w/w) poloxamer hydrogel (e.g., 18% (w/w) P407 hydrogel). In some embodiments, a provided polymer combination preparation may have a comparable (e.g., within 20%, or within 15%, or within 10%, or within 5%) 24-hour release profile (e.g., percent release over a period of 48 hours) of an incorporated lipophilic agent as that for an 18% (w/w) poloxamer hydrogel (e.g., 18% (w/w) P407 hydrogel).

In some embodiments, a polymer network state of a provided polymer combination preparation is characterized in that, when tested in vitro at 37° C., no more than 40% or less, including, e.g., no more than 35%, no more than 30%, no more than 25%, no more than 20%, no more than 15%, no more than 10%, no more than 5% or less, of a lipophilic agent incorporated in the polymer combination preparation is released therefrom within 24 hours. In some embodiments, a polymer network state of a provided polymer combination preparation is characterized in that, when tested in vitro at 37° C., no more than 40% or less, including, e.g., no more than 35%, no more than 30%, no more than 25%, no more than 20%, no more than 15%, no more than 10%, no more than 5% or less, of a lipophilic agent incorporated in the polymer combination preparation is released therefrom within 48 hours.

In some embodiments, a polymer network state of a provided polymer combination preparation is characterized in that, when tested in vitro at 37° C., 50% or more, including, e.g., more than 55%, more than 60%, more than 65%, more than 70%, more than 75%, more than 80%, more than 85%, more than 90%, more than 95%, or more, of a lipophilic agent incorporated in the polymer combination preparation can be retained therein for at least 24 hours. In some embodiments, a polymer network state of a provided polymer combination preparation is characterized in that, when tested in vitro at 37° C., 50% or more, including, e.g., more than 55%, more than 60%, more than 65%, more than 70%, more than 75%, more than 80%, more than 85%, more than 90%, more than 95%, or more, of a lipophilic agent incorporated in the polymer combination preparation is retained therein for at least 48 hours.

In some embodiments, a polymer network state of a provided polymer combination preparation is characterized in that, when tested in vitro at 37° C., such a polymer combination preparation releases a hydrophilic agent incorporated therein at a comparable rate (e.g., within 20%, within 15%, within 10%, or within 5%) as or at a faster rate than that of an 18% (w/w) poloxamer hydrogel (e.g., 18 wt% P407 hydrogel), for example, as measured over a period of 24 hours or longer (e.g., 24 hours, 48 hours, or longer). In some embodiments, such a polymer combination preparation is characterized in that, when tested in vitro at 37° C., it releases a hydrophilic agent incorporated therein at a faster rate by at least 5% or more, including, e.g., at least 10%, at least 15%, at least 20%, at least 25%, at least 30%, or more, than that of an 18 wt% poloxamer hydrogel (e.g., 18% (w/w) P407 hydrogel), for example, as measured over a period of 24 hours or longer. In some embodiments, such a polymer combination preparation is characterized in that, when tested in vitro at 37° C., it releases a hydrophilic agent incorporated therein at a faster rate by at least 5% or more, including, e.g., at least 10%, at least 15%, at least 20%, at least 25%, at least 30%, or more, than that of an 18% (w/w) poloxamer hydrogel (e.g., 18% (w/w) P407 hydrogel), for example, as measured over a period of 48 hours.

In some embodiments, a polymer network state of a provided polymer combination preparation is characterized in that, when tested in vitro at 37° C., such a polymer combination preparation releases a hydrophilic agent incorporated therein at a faster rate, as compared to a reference chemically crosslinked hydrogel, for example, as measured over a period of 24 hours or longer (e.g., 24 hours, 48 hours, or longer). In some embodiments, such a polymer combination preparation is characterized in that, when tested in vitro at 37° C., it releases a hydrophilic agent incorporated therein at a faster rate by at least 5% or more, including, e.g., at least 10%, at least 15%, at least 20%, at least 25%, at least 30%, or more, as compared to a reference chemically crosslinked hydrogel, for example, as measured over a period of 24 hours or longer. In some embodiments, such a polymer combination preparation is characterized in that, when tested in vitro at 37° C., it releases a hydrophilic agent incorporated therein at a faster rate by at least 5% or more, including, e.g., at least 10%, at least 15%, at least 20%, at least 25%, at least 30%, or more, as compared to a reference chemically crosslinked hydrogel, for example, as measured over a period of 48 hours. For comparison purposes, in some embodiments, a reference chemically crosslinked hydrogel may be a chemically crosslinked hyaluronic acid hydrogel, which in some embodiments may be prepared by mixing thiol-modified hyaluronic acid (Glycosil®) with a chemical crosslinking agent, thiol-reactive PEGDA crosslinker (Extralink®) under conditions for gelation to occur.

In some embodiments, a polymer network state of a provided polymer combination preparation is characterized in that, when tested in vitro at 37° C., at least 20% or more, including, e.g., at least 25%, at least 30%, at least 35%, at least 40%, at least 45%, at least 50% or more, of a hydrophilic agent incorporated therein is released over a period of 12 hours or longer. In some embodiments, a polymer network state of a provided polymer combination preparation is characterized in that, when tested in vitro at 37° C., at least 30% or more, including, e.g., at least 35%, at least 40%, at least 45%, at least 50%, at least 55%, at least 60% or more, of a hydrophilic agent incorporated therein is released over a period of 24 hours or longer. In some embodiments, a polymer network state of a provided polymer combination preparation is characterized in that, when tested in vitro at 37° C., at least 40% or more, including, e.g., at least 45%, at least 50%, at least 55%, at least 60%, at least 65%, at least 70% or more, of a hydrophilic agent incorporated therein is released over a period of 48 hours or longer.

### G. In Vivo Efficacy

In some embodiments, a composition of the present disclosure comprises an immunomodulatory polymer combination preparation, which comprises a poloxamer (e.g., ones described herein) and at least one carbohydrate polymer (e.g., described herein), which preparation is substantially free of an immunomodulatory payload (e.g., as described herein). In some embodiments, such a composition and/or an immunomodulatory polymer combination preparation may induce innate immunity agonism. In some embodiments, such a composition and/or an immunomodulatory polymer combination preparation may resolve or reduce inflammation, which in some embodiments may be or comprise immunosuppressive inflammation. In some embodiments, such a composition and/or an immunomodulatory polymer combination preparation included in the composition is characterized in that a test animal group with spontaneous metastases having, at a tumor resection site, such a polymer combination preparation in a polymer network state has a higher percent survival than that of a comparable test animal group having, at a tumor resection site, a poloxamer biomaterial (e.g., a 15-18% (w/w) P407 biomaterial), as assessed at 2 months after the administration. In some such embodiments, an increase in percent survival as observed in a test animal group with spontaneous metastases having, at a tumor resection site, a polymer combination preparation (e.g., ones described herein) is at least 30% or more, including, at least 40%, at least 50%, at least 60%, at least 70%, at least 80%, at least 90%, or more, as compared to that of a comparable test animal group having, at a tumor resection site, a poloxamer biomaterial (e.g., a 15-18% (w/w) P407 biomaterial), as assessed at 2 months after the administration.

In some embodiments, at least one therapeutic agent (e.g., at least one immunomodulatory payload as described herein) may be incorporated in a polymer combination preparation and/or composition comprising the same described herein. In some embodiments, such a polymer combination preparation is characterized in that a test animal group with spontaneous metastases having, at a tumor resection site, the polymer combination preparation in a polymer network state has a higher percent survival than that of a comparable test animal group having, at a tumor resection site, the same polymer combination preparation without the immunomodulatory payload, as assessed at 2 months after the administration. In some such embodiments, an increase in percent survival as observed in a test animal group with spontaneous metastases having, at a tumor resection site, a provided polymer combination preparation (incorporating an immunomodulatory payload) is at least 30% or more, including, at least 40%, at least 50%, at least 60%, at least 70%, at least 80%, at least 90%, or more, as compared to that of a comparable test animal group having, at a tumor resection site, the same polymer combination preparation without the immunomodulatory payload, as assessed at 2 months after the administration.

### III. Exemplary Embodiments of Provided Polymer Combination Preparations

In some embodiments, polymer combination preparations described herein are prepared in a phosphate buffer or a carbonate buffer at pH 7-8. In some embodiments, a phosphate buffer may have a concentration of 10-50 mM (including, e.g., 10 mM, 20 mM, 30 mM, 40 mM, or 50 mM). In some embodiments, a bicarbonate buffer may have a concentration of 25- 200 mM (including, e.g., 25 mM, 50 mM, 75 mM, 100 mM, 125 mM, 150 mM, 175 mM, or 200 mM).

In some embodiments, polymer combination preparations described herein are temperature-responsive, and have a critical gelation temperature of about 10-30° C. In some embodiments, such polymer combination preparations described herein may have a critical gelation temperature of around room temperature, e.g., 10-15° C. In some embodiments, such polymer combination preparations described herein may have a critical gelation temperature of around room temperature, e.g., 15-20° C. In some embodiments, such polymer combination preparations described herein may have a critical gelation temperature of around room temperature, e.g., 20-25° C. In some embodiments, such polymer combination preparations described herein may have a critical gelation temperature of about 25-28° C. In some embodiments, such polymer combination preparations described herein may have a critical gelation temperature of about 28-32° C. In some embodiments, such polymer combination preparations described herein may have a critical gelation temperature of about 32-34° C. In some embodiments, such polymer combination preparations described herein may have a critical gelation temperature of about 34-37° C.

In certain embodiments, a polymer combination preparation comprises 5-12.5% (w/w) or 6-10% (w/w) Poloxamer 407 and 0.5-3% (w/w) hyaluronic acid having an average molecular weight of 1-2 MDa. In some embodiments, such a polymer combination preparation (e.g., upon transition to a polymer network state) may be characterized by a storage modulus, which may range from approximately 300 Pa to approximately 4,600 Pa or approximately 300 Pa to approximately 6,500 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-42,00 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, or approximately 5,800-6,500 Pa). In some embodiments, such a polymer combination preparation (e.g., upon transition to a polymer network state) may be characterized by a phase angle of about 2-20°.

In certain embodiments, a polymer combination preparation comprises 5-12.5% (w/w), 7-12.5% (w/w), 7-11.5% (w/w), 6-11.5% (w/w), 5-11.5% (w/w), 5-11% (w/w), 5-10.5% (w/w), 6-10.5% (w/w), 6-10% (w/w), 7-11% (w/w), or 8-11% (w/w) Poloxamer 407, with 0.5-3% (w/w) hyaluronic acid, 0.5-2% (w/w) hyaluronic acid, 1-2% (w/w) hyaluronic acid, 1-3% (w/w) hyaluronic acid, 1-4% (w/w) hyaluronic acid, 2-5% (w/w) hyaluronic acid, 3-6% (w/w) hyaluronic acid, or 4-7% (w/w) hyaluronic acid having an average molecular weight of 500 kDa-900 kDa. In some embodiments, such a polymer combination preparation (e.g., upon transition to a polymer network state) may be characterized by a storage modulus, which may range from approximately 100 Pa to approximately 7,600 Pa, 100 Pa to approximately 15,000 Pa, or 500 Pa to approximately 18,000 Pa. In some embodiments, such a polymer combination preparation (e.g., upon transition to a polymer network state) may be characterized by a storage modulus, which may range from approximately 300 Pa to approximately 8,000 Pa, (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-42,00 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,600 Pa, approximately 6,400-6,800 Pa, approximately 6,600-7,000 Pa, approximately 6,800-7,200 Pa, approximately 7,000-7,400 Pa, approximately 7,200-7,600 Pa, approximately 7,400-7,800 Pa, approximately 7,600-8,000 Pa). In some embodiments, such a polymer combination preparation (e.g., upon transition to a polymer network state) may be characterized by a phase angle of about 2-20°.

In certain embodiments, a polymer combination preparation including a high MW hyaluronic acid comprises a formulation described in Example 2, Table 3.

In certain embodiments, a polymer combination preparation comprises 5-12.5% (w/w), 8-12.5% (w/w), 6-11.5% (w/w), 6-11% (w/w), 7-11% (w/w), or 8-11% (w/w), 6-10.5% (w/w), or 6-10% (w/w) Poloxamer 407, with 1-4% (w/w) hyaluronic acid, 2-5% (w/w) hyaluronic acid, 1-10% (w/w) hyaluronic acid, or 1.5-10% (w/w) hyaluronic acid, or 3-6% (w/w) hyaluronic acid, or 4-7% (w/w) hyaluronic acid having an average molecular weight of 100 kDa-500 kDa. In certain embodiments, a polymer combination preparation comprises 5-12.5% (w/w), 8-12.5% (w/w), 8-11% (w/w), 6-11% (w/w), 6-10.5% (w/w), or 6-10% (w/w) Poloxamer 407, and 0.5-10% (w/w) hyaluronic acid, or 1.5-10% (w/w) hyaluronic acid, or 2-6% (w/w) hyaluronic acid or 4-9% (w/w) hyaluronic acid having an average molecular weight of 100 kDa-300 kDa. In certain embodiments, a polymer combination preparation comprises 8-12.5% (w/w) or 8-11% (w/w) Poloxamer 407 and 2-6% (w/w) hyaluronic acid having an average molecular weight of 100 kDa-200 kDa. In some embodiments, such a polymer combination preparation (e.g., upon transition to a polymer network state) may be characterized by a storage modulus, which may range from approximately 400 Pa to approximately 3,400 Pa. In some embodiments, such a polymer combination preparation (e.g., upon transition to a polymer network state) may be characterized by a phase angle of about 2-35° or 2-20°. In certain embodiments, a polymer combination preparation comprises 5-11% (w/w), 6-10.5% (w/w), or 6-10% (w/w) Poloxamer 407 and 1-10% (w/w) or 1.5-10% (w/w) hyaluronic acid having an average molecular weight of 100 kDa-200 kDa. In some embodiments, such a polymer combination preparation (e.g., upon transition to a polymer network state) may be characterized by a storage modulus, which may range from approximately 400 Pa to approximately 5,000 Pa or 300 Pa to approximately 6,500 Pa, (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-42,00 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,500 Pa). In some embodiments, such a polymer combination preparation (e.g., upon transition to a polymer network state) may be characterized by a phase angle of about 20-35°.

In certain embodiments, a polymer combination preparation including a low MW hyaluronic acid comprises a formulation described in Example 2, Table 2.

In certain embodiments, a polymer combination preparation comprises 8-12.5% (w/w) or 8-11% (w/w) Poloxamer 407 or 6-10% (w/w) Poloxamer 407, and 1-10% (w/w), or 1.5-9% (w/w) or 1-5% (w/w) or 5-10% (w/w) hyaluronic acid having an average molecular weight of 70 kDa -200 kDa or 80 kDa-150 kDa. In some embodiments, such a polymer combination preparation (e.g., upon transition to a polymer network state) may be characterized by a storage modulus, which may range from approximately 200 Pa to approximately 6,500 Pa, or approximately 200 Pa to approximately 5,900 Pa. In some embodiments, such a polymer combination preparation (e.g., upon transition to a polymer network state) may be characterized by a phase angle of about 2-35° or 2-20°. In some embodiments, such a polymer combination preparation (e.g., upon transition to a polymer network state) may be characterized by a storage modulus, which may range from approximately 400 Pa to approximately 6,500 Pa, or 400 Pa to approximately 4,600 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa, approximately 3,200-3,600 Pa, approximately 3,400-3,800 Pa, approximately 3,600-4,000 Pa, approximately 3,800-4,200 Pa, approximately 4,000-4,400 Pa, approximately 4,200-4,600 Pa, approximately 4,400-4,800 Pa, approximately 4,600-5,000 Pa, approximately 4,800-5,200 Pa, approximately 5,000-5,400 Pa, approximately 5,200-5,600 Pa, approximately 5,400-5,800 Pa, approximately 5,600-6,000 Pa, approximately 5,800-6,200 Pa, approximately 5,800-6,400 Pa, approximately 6,000-6,400 Pa, approximately 6,200-6,500 Pa). In some embodiments, such a polymer combination preparation (e.g., upon transition to a polymer network state) may be characterized by a phase angle of about 2-32°, or about 15 to 35°.

In certain embodiments, a polymer combination preparation comprises 8-12.5% (w/w) or 8-11% (w/w) Poloxamer 338 and 1-3% (w/w) hyaluronic ac id having an average molecular weight of 1-2 MDa. In some embodiments, such a polymer combination preparation (e.g., upon transition to a polymer network state) may be characterized by a storage modulus, which may range from approximately 980 Pa to approximately 1,300 Pa. In some embodiments, such a polymer combination preparation (e.g., upon transition to a polymer network state) may be characterized by a phase angle of about 2-35° or 2-20°.

In certain embodiments, a polymer combination preparation comprises 5-12.5% (w/w) or 8-11.5% (w/w), or 8-11% (w/w) Poloxamer 338, with 1-4% (w/w) hyaluronic acid having an average molecular weight of 500 kDa-900 kDa. In some embodiments, such a polymer combination preparation (e.g., upon transition to a polymer network state) may be characterized by a storage modulus, which may range from approximately 1,400 Pa to approximately 2,700 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 338 and 1-5% (w/w) hyaluronic acid having an average molecular weight of 100 kDa-350 kDa. In some embodiments, such a polymer combination preparation (e.g., upon transition to a polymer network state) may be characterized by a storage modulus, which may range from approximately 500 Pa to approximately 1,350 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407 and 2.5-5% (w/w) modified chitosan (e.g., carboxymethyl chitosan). In some embodiments, such a polymer combination preparation (e.g., upon transition to a polymer network state) may be characterized by a storage modulus, which may range from approximately 1,000 Pa to approximately 5,000 Pa. In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 0.5-5% (w/w) hyaluronic acid having an average molecular weight of 500 kDa-900 kDa, and 0.1-1.5% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) or 6-11% (w/w) or 6-10.5% (w/w) or 6-10% (w/w) Poloxamer 407, 0.5%-10% (w/w) or 1-10% (w/w) or 1-5% (w/w) hyaluronic acid having an average molecular weight of 80 kDa-150 kDa, and 0.1-5% (w/w) or 0.2-5% (w/w) or 0.1-3% (w/w) modified chitosan (e.g., carboxymethyl chitosan and/or chitosan-phenyl succinic acid)). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 1-5% (w/w) hyaluronic acid having an average molecular weight of 500 kDa-900 kDa, and 0.2-4% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-,1400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 8-12.5% (w/w) or 8-11% (w/w) Poloxamer 407, 1-5% (w/w) hyaluronic acid having an average molecular weight of 100 kDa-500 kDa, and 0.2-4% modified chitosan (e.g., carboxymethyl chitosan). In some embodiments, such a polymer combination preparation (e.g., upon transition to a polymer network state) may be characterized by a storage modulus, which may range from approximately 400 Pa to approximately 3,400 Pa (e.g., approximately 400 to 800 Pa, approximately 600-1,000 Pa, approximately 800-1,200 Pa, approximately 1,000-1,400 Pa, approximately 1,200-1,600 Pa, approximately 1,400-1,800 Pa, approximately 1,600-2,000 Pa, approximately 1,800-2,200 Pa, approximately 2,000-2,400 Pa, approximately 2,200-2,600 Pa, approximately 2,400-2,800 Pa, approximately 2,600-3,000 Pa, approximately 2,800-3,200 Pa, approximately 3,000-3,400 Pa). In some embodiments, such a polymer combination preparation (e.g., upon transition to a polymer network state) may be characterized by a phase angle of about 2-35° or 2-20°.

In certain embodiments, a polymer combination preparation comprises 3.5-5.5% (w/w) or 4-5% (w/w) Poloxamer 407, and 1.5-3.5% (w/w) high molecular weight hyaluronic acid (e.g., hyaluronic acid having an average molecular weight of greater than 500 kDa, such as, e.g., 600-1500 kDa, or 700-1500 kDa). In some embodiments, such a polymer combination preparation (e.g., upon transition to a polymer network state) may be characterized by a storage modulus, which may range from approximately 200 Pa to approximately 10,000 Pa, 500 Pa to approximately 9,000 Pa, or approximately 1,000 Pa to approximately 8,000 Pa, or 1,000 Pa to approximately 6,000 Pa.

In some embodiments, such polymer combination preparations may incorporate a payload, e.g., an immunomodulatory payload.

### IV. Exemplary Methods for Preparing Polymer Combination Preparations

In some embodiments, polymer combination preparations described herein may be prepared by mixing an appropriate amount of poloxamer and at least a second polymer component (e.g., hyaluronic acid and/or chitosan) in an appropriate buffer. Poloxamer and at least a second polymer component (e.g., hyaluronic acid and/or chitosan) may be independently a solid particle preparation or a liquid preparation. In some embodiments, a payload, e.g., an immunomodulatory payload in some embodiments, may be added to such a polymer mixture solution. In some embodiments, a polymer mixture solution may be mixed at a low speed (e.g., a speed of less than 100 rpm until a homogenous polymer solution is formed. To induce gel formation, such a homogenous polymer solution can be exposed to a critical gelation temperature or above for a period of time sufficient for gel formation (e.g., 10-15 mins).

In some embodiments, the present disclosure, among other things, provides an insight that mixing a solid particle preparation of hyaluronic acid (HA) with at least a second polymer preparation (e.g., poloxamer), which may be a solid particle preparation or a liquid preparation, can promote formation of a homogenous polymer solution, as compared to mixing liquid preparations of HA and at least a second polymer.

Accordingly, one aspect provided herein relates to a method of producing a homogenous polymer combination of a hyaluronic acid (HA) polymer preparation and a second polymer preparation. The method comprises a step of combining a HA and a second polymer preparation when the HA polymer preparation is in solid particle form. In some embodiments, a solid particle preparation of HA polymer comprises HA polymer in powder form. One of skilled in the art, reading the present disclosure, will understand that HA polymer tends to be hygroscopic; in some embodiments, HA polymer in a solid particle preparation may be or comprise hydrated HA polymer.

In some embodiments, a HA polymer preparation in solid particle form may be combined with at least a second polymer preparation (e.g., poloxamer) in solid particle form (e.g., powder in some embodiments) and then together dissolved concurrently in a liquid solution (e.g., a buffer). In some embodiments, a HA polymer preparation in solid particle form may be combined with at least a second polymer preparation (e.g., poloxamer) in liquid form, which in some embodiments may be a solution of the second polymer in a solvent system (e.g., as described herein).

In some embodiments, such HA and second polymer preparations, and optionally additional polymer preparation(s), are combined under conditions and for a time sufficient so that a homogenous polymer mixture is produced. In some embodiments, such a produced homogenous polymer mixture is characterized in that there is no detectable phase separation observed after maintaining the produced homogenous polymer mixture at a temperature that is below of the critical gelation temperature (e.g., in some embodiments 2-8° C. or in some embodiments at an ambient temperature) for at least 1 hour or longer, including, e.g., at least 2 hours, at least 3 hours, at least 4 hours, at least 5 hours, at least 6 hours, at least 12 hours, at least 18 hours, at least 24 hours, or longer. In some embodiments, such a produced homogenous polymer mixture is characterized in that there is no detectable phase separation observed after maintaining the produced homogenous polymer mixture at a temperature that is below of the critical gelation temperature (e.g., in some embodiments 2-8° C. or in some embodiments at an ambient temperature) for at least 1 week or longer, including, e.g., at least 2 weeks, at least 3 weeks, at least 4 weeks, longer. In some embodiments, such a produced homogenous polymer mixture is characterized in that there is no detectable phase separation observed after maintaining the produced homogenous polymer mixture at a temperature that is below of the critical gelation temperature (e.g., in some embodiments 2-8° C. or in some embodiments at an ambient temperature) for at least 1 month or longer, including, e.g., at least 2 months, at least 3 months, or longer.

In some embodiments, a HA and a second polymer preparations (e.g., poloxamer), and optionally additional polymer preparation(s), are combined by mixing them at an ambient temperature and/or at a low shear rate. In some embodiments, mixing may be performed by mechanical stirring. As will be understood by one skilled in the art, the shear rate is typically determined by the dimensions of a stirring unit (e.g., a stirred blade or stirrer bar such as a magnetic stirrer bar) and rpm, and the highest shear is typically at the tips of a stirring unit (e.g., stirrer blade or a stirrer bar). In some embodiments, a cylindrical stirrer bar, which induces radial flow, may be used. In some embodiments, an impeller of at least 2 blades (e.g., 2, 3, or 4 blades) may be used to induce axial or radial flow depending on the geometry of the blades. In axial flow, the motion is parallel to the shaft (down and up); in radial flow, the motion is perpendicular to the shaft. In some embodiments, a HA and a second polymer preparations are combined by mixing them at an ambient temperature and at a speed of less than 100 rpm.

In some embodiments, a HA and a second polymer preparations (e.g., poloxamer), and optionally additional polymer preparation(s), are mixed for a period of at least 5 hours, including, e.g., at least 10 hours, at least 15 hours, at least 20 hours, at least 25 hours, at least 30 hours or longer. In some embodiments, a HA and a second polymer preparations, and optionally additional polymer preparation(s) are mixed for a period of 5-30 hours or 10-24 hours.

In some embodiments, a HA and a second polymer preparations (e.g., poloxamer), and optionally additional polymer(s), may be mixed at a temperature of between 2-8° C., for example, in some embodiments for a period of at least 5 hours, including, e.g., at least 10 hours, at least 15 hours, at least 20 hours, at least 25 hours, at least 30 hours or longer.

In some embodiments, a HA and a second polymer preparations (e.g., poloxamer), and optionally additional polymer(s) (e.g., CMCH), are mixed at a temperature of between 2-8° C. and are then quickly brought to a temperature that is at or greater than the respective CGT (e.g., relevant CGTs as described herein) to reach a polymer network state, for example, to prevent phase separation. In some such embodiments, a resulting polymer network may be stored at a temperature that is at or greater than the respective CGT (e.g., relevant CGTs as described herein), for example in some embodiments at an ambient temperature, until it is ready for delivery. In some embodiments, a resulting polymer network may be delivered at a temperature that is lower than the respective CGT (e.g., relevant CGTs as described herein) to render it as a solution and/or liquid preparation.

In some embodiments, a payload (e.g., ones described herein) may be incorporated into a homogenous mixture of HA and second polymer preparations. In some embodiments, a payload may be added by combining a HA and a second polymer preparations with a payload. In some embodiments, a payload to be combined may be a solid particle preparation. In some embodiments, a payload to be combined may be a liquid preparation.

In some embodiments, a produced homogenous polymer mixture (with or without a payload) may be exposed to a gelation temperature that is or above the critical gelation temperature of the polymer mixture for a time sufficient so that a hydrogel is formed. In some embodiments, a produced homogenous polymer mixture (with or without a payload) may be exposed to a gelation temperature of about 35-39° C. In some embodiments, a produced homogenous polymer mixture (with or without a payload) may be exposed to a gelation temperature of about 37° C. In some embodiments, a produced homogenous polymer mixture (with or without a payload) is exposed to a gelation temperature for a period of 5 minutes to 30 minutes.

### V. Pharmaceutical Compositions

In some embodiments, a provided polymer combination preparation and/or composition can be formulated in accordance with routine procedures as a pharmaceutical composition for administration to a subject in need thereof (e.g., as described herein). In some embodiments, such a pharmaceutical composition can include a pharmaceutically acceptable carrier or excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington’s The Science and Practice of Pharmacy, 21st Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, lactose, trehalose, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In some embodiments, a pharmaceutical composition can be sterile. A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a pharmaceutical composition for injection may typically comprise sterile isotonic aqueous buffer. Where necessary, a pharmaceutical composition may also include a local anesthetic to ease pain at a site of injection. In some embodiments, components of a pharmaceutical composition (e.g., as described herein) are supplied separately or mixed together in a single-use form, for example, as a dry lyophilized powder or water free concentrate in a hermetically sealed container such as an ampule or sachet or in a sterile syringe indicating the quantity of a composition comprising a polymer combination preparation (e.g., ones described herein). Where a pharmaceutical composition is to be administered by injection, in some embodiments, a dry lyophilized powder composition comprising a polymer combination preparation (e.g., ones described herein) can be reconstituted with an aqueous buffered solution and then injected to a target site in a subject in need thereof. In some embodiments, a liquid composition comprising a polymer combination preparation (e.g., ones described herein) can be provided in a syringe for administration by injection and/or by a robotic surgical system (e.g., a da Vinci System).

In some embodiments, a liquid composition comprising a polymer combination preparation (e.g., ones described herein) can be provided in a syringe for administration with or without a needle, cannula, or trocar.

In some embodiments, a liquid composition comprising a polymer combination preparation (e.g., ones described herein) can be administered by spraying.

In some embodiments, administration of a liquid composition comprising a polymer combination preparation (e.g., ones described herein) can be gas assisted for use in minimally invasive surgery.

In some embodiments, administration of a liquid composition comprising a polymer combination preparation (e.g., ones described herein) can be achieved by using a multi-barrel syringe, with each barrel containing a separate polymer component preparation, the multiple of which are combined upon depression of the shared plunger.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. For example, such preparatory methods include step of bringing components of a provided polymer combination preparation, optionally with a payload such as a therapeutic agent, into association with a diluent or another excipient and/or one or more other accessory ingredients and then, if necessary and/or desirable, shaping and/or packaging the product into a desired single-use unit or multi-use units. Alternatively, such preparatory methods may also include a step of pre-forming a polymer network biomaterial from components of a polymer combination preparation described herein, prior to shaping and/or packaging the product into a desired single-use units or multi-use units.

A pharmaceutical composition in accordance with the present disclosure may be prepared, packaged, and/or sold in bulk, as a single-use unit, and/or as a plurality of single-use units. As used herein, a “single-use unit” is a discrete amount of a pharmaceutical composition described herein. For example, a single-use unit of a pharmaceutical composition comprises a predetermined amount of a composition and/or polymer combination preparation described herein, which in some embodiments can be or comprise a pre-formed polymer network of a polymer combination preparation (e.g., ones described herein), or in some embodiments can be or comprise a liquid or a colloidal mixture of individual components of a polymer combination preparation (e.g., ones described herein).

The relative amount of individual components of a provided polymer combination preparation (e.g., as a pre-formed polymer network biomaterial or as precursor component(s) of such a polymer network biomaterial) and, optionally, any additional agents in pharmaceutical compositions described herein, e.g., a pharmaceutically acceptable excipient and/or any additional ingredients, can vary, depending upon, e.g., desired material properties of a polymer biomaterial, size of target site, injection volume, physical and medical condition of a subject to be treated, and/or types of cancer, and may also further depend upon the route by which such a pharmaceutical composition is to be administered. In some embodiments, a polymer combination preparation and optionally a payload (e.g., a therapeutic agent as described herein) is provided in an effective amount in a pharmaceutical composition to provide a desired therapeutic effect (e.g., but not limited to inducing anti-tumor immunity in at least one or more aspects, e.g., inducing innate immunity). In some embodiments, a polymer combination preparation and optionally a payload (e.g., a therapeutic agent as described herein) is provided in an effective amount in a pharmaceutical composition for treatment of cancer. In some embodiments, a polymer combination preparation and optionally a payload (e.g., a therapeutic agent as described herein) is provided in an effective amount in a pharmaceutical composition to inhibit or reduce risk or incidence of tumor recurrence and/or metastasis. In certain embodiments, the effective amount is a therapeutically effective amount of a polymer combination preparation and optionally a payload (e.g., a therapeutic agent as described herein). In certain embodiments, the effective amount is a prophylactically effective amount of a polymer combination preparation and optionally a payload (e.g., a therapeutic agent as described herein).

In certain embodiments, a pharmaceutical composition consists essentially of or consists of a polymer combination preparation (e.g., ones described herein); to the extent that such a composition may include one or more material(s)/agents other than the polymer combination preparation, such other material(s)/agent(s) do not, individually, or together, materially alter relevant immunomodulatory characteristic(s), e.g., innate immunity modulatory characteristic(s) of the polymer combination preparation. In some embodiments, such a pharmaceutical composition may be substantially free of an immunomodulatory payload.

In certain embodiments, pharmaceutical compositions do not include cells. In certain embodiments, pharmaceutical compositions do not include adoptively transferred cells. In certain embodiments, pharmaceutical compositions do not include T cells. In certain embodiments, pharmaceutical compositions do not include tumor antigens. In certain embodiments, pharmaceutical compositions do not include tumor antigens loaded ex vivo.

In certain embodiments, a pharmaceutical composition is in liquid form (e.g., a solution or a colloid). In certain embodiments, a pharmaceutical composition is in a solid form (e.g., a gel form). In certain embodiments, the transition from a liquid form to a solid form may occur outside a subject’s body upon sufficient crosslinking such that the resulting material has a storage modulus consistent with a solid form that allows it to be physically manipulated and implanted in a surgical procedure. Accordingly, in some embodiments, a solid form may be amenable for carrying out an intended use of the present disclosure (e.g., surgical implantation). In certain embodiments, the transition from a liquid form to a solid form may occur upon thermal crosslinking in situ (e.g., inside a body of a subject) such that the resulting material has a storage modulus consistent with a solid form. In certain embodiments, a pharmaceutical composition is a suspension.

### VI. Therapeutic Uses

In many embodiments, polymer combination preparations and compositions comprising the same described herein are biocompatible and are useful for various medical applications, e.g., in some embodiments as a drug delivery carrier or formulation (e.g., sustained-release drug delivery composition). For example, in some embodiments, polymer combination preparations and compositions comprising the same described herein are useful for treatment of a disease, disorder, or condition. In some embodiments, polymer compositions and compositions comprising the same described herein are useful for treatment of cancer. In some embodiments, polymer combination preparations and compositions comprising the same described herein are useful to delay the onset of, slow the progression of, or ameliorate one or more symptoms of cancer. In some embodiments, polymer combination preparations and compositions comprising the same described herein are useful to reduce or inhibit primary tumor regrowth. In some embodiments polymer combination preparations and compositions comprising the same described herein reducing or inhibiting incidence of tumor recurrence and/or metastasis. In some embodiments, polymer combination preparations and compositions comprising the same described herein are useful for inducing anti-tumor immunity. For example, in some embodiments, a polymer combination preparation described herein by itself is sufficient to provide anti-tumor immunity (e.g., in some embodiments by inducing innate immunity agonism in a subject) without necessarily requiring incorporation of an immunomodulatory payload. In some embodiments, a polymer combination preparation described herein may incorporate one or more immunomodulatory payloads to provide or augment antitumor immunity (e.g., in some embodiments by inducing innate immunity in a subject). In some embodiments, a polymer combination preparation described herein may incorporate one or more therapeutic agents (e.g., one or more chemotherapeutic agents).

Accordingly, some aspects provided herein relate to methods of administering to a target site in a subject in need thereof a composition comprising a polymer combination preparation described herein. In some embodiments, a subject receiving such a composition may be carrying a tumor. In some such embodiments, a method comprises intratumoral or peritumoral administration of a composition comprising a polymer combination preparation described herein. In some embodiments, a subject receiving such a composition may be undergoing or may have undergone tumor removal (e.g., by surgical tumor resection). In some embodiments, a subject receiving such a composition may have tumor relapse and/or metastasis. In some such embodiments, a method comprises intraoperative administration of a composition comprising a polymer combination preparation described herein at a tumor resection site of a subject.

In some embodiments, a composition administered to a subject in need thereof consists essentially of or consists of a polymer combination preparation having immunomodulatory characteristic (e.g., having a characteristic of inducing innate immunity) without any immunomodulatory payload; to the extent that such a composition may include one or more material(s)/agent(s) other than the polymer combination preparation, such other material(s)/agent(s) do not, individually or together, materially alter relevant immunomodulatory characteristic(s) (e.g., innate immunity modulatory characteristic(s) of the polymer combination preparation. In some embodiments, such a provided composition may comprise a polymer combination preparation (e.g., ones described herein) that is substantially free of an immunomodulatory payload. In some embodiments, such a provided composition may be substantially free of any immunomodulatory payload. In some embodiments, such a provided composition utilized in methods of the present disclosure may be formulated as a pharmaceutical composition described herein.

In some embodiments, a composition administered to a subject in need thereof comprises a polymer combination preparation and a therapeutic agent, which may be in some embodiments a chemotherapeutic agent, while in some embodiments an immunomodulatory agent). In some embodiments, a composition administered to a subject in need thereof comprises a provided polymer combination preparation and one or more immunomodulatory agents as described in International Patent Publication No. WO 2018/045058 and WO 2019/183216, the contents of each of which are incorporated herein by reference for purposes described herein. In some embodiments, a composition administered to a subject in need thereof comprises a polymer combination preparation described herein and an activator of innate immune response, for example, in some embodiments, which may be or comprise a stimulator of interferon genes (STING) agonist, a Toll-like receptor (TLR) agonist, and/or an activator of innate immune response as described in International Patent Publication No. WO 2018/045058, the contents of which are incorporated herein by reference for purposes described herein. In some embodiments, a composition administered to a subject in need thereof comprises a polymer combination preparation described herein and an inhibitor of immunosuppressive inflammation, for example, in some embodiments, which may be or comprise a COX2 inhibitor or an agent that inhibits one or more proinflammatory pathways, such as one or more immune responses mediated by a p38 mitogen-activated protein kinase (MAPS) pathway, as described in International Patent Publication No. WO 2019/183216, the contents of which are incorporated herein by reference for purposes described herein. In some embodiments, such a provided composition utilized in methods of the present disclosure may be formulated as a pharmaceutical composition described herein.

In certain embodiments, a method provided herein comprises administering a provided composition to a target site in a subject in need thereof after removal of tumor, for example, after removal of greater than or equal to 50% or higher, by weight, of the subject’s tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by weight, of the subject’s tumor. In certain embodiments, a method provided herein comprises administering a provided composition to a target site in a subject in need thereof after removal of greater than or equal to 50% or higher, by volume, of the subject’s tumor, including, e.g., greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, greater than or equal to 96%, greater than or equal to 97%, greater than or equal to 98%, or greater than or equal to 99%, by volume, of the subject’s tumor. In some embodiments, a method provided herein comprises performing a tumor resection to remove a subject’s tumor, prior to administration of a provided composition.

In some embodiments, a composition described and/or utilized herein is administered to a target site in a tumor resection subject immediately after the subject’s tumor has been removed by surgical tumor resection. In some embodiments, a composition described and/or utilized herein is intraoperatively administered to a target site in a tumor section subject. In some embodiments, a composition described and/or utilized herein is postoperatively administered to a target site in a tumor resection subject within 24 hours or less, including, e.g., within 18 hours, within 12 hours, within 6 hours, within 3 hours, within 2 hours, within 1 hour, within 30 mins, or less, after the subject’s tumor has been removed by surgical tumor resection. In some embodiments, a composition described and/or utilized herein is postoperatively administered one or more times to one or more target sites at one or more time points within 12 months or less from a surgical intervention, including e.g., within 11 months, within 10 months, within 9 months, within 8 months, within 7 months, within 6 months, within 5 months, within 4 months, within 3 months, within 2 months, or within 1 months of a surgical intervention. In some embodiments, a composition described and/or utilized herein is postoperatively administered one or more times to one or more target sites at one or more time points within 31 days, including e.g., within 30 days, within 29 days, within 28 days, within 27 days, within 26 days, within 25 days, within 24 days, within 23 days, within 22 days, within 21 days, within 20 days, within 19 days, within 18 days, within 17 days, within 16 days, within 15 days, within 14 days, within 13 days, within 12 days, within 11 days, within 10 days, within 9 days, within 8 days, within 7 days, within 6 days, within 5 days, within 4 days, within 3 days, within 2 days, or within 1 day of a surgical intervention.

In some embodiments, a target site for administration is or comprises a tumor resection site. In some embodiments, such a tumor resection site may be characterized by absence of gross residual tumor antigen. In some embodiments, such a tumor resection site may be characterized by a negative resection margin (i.e., no cancer cells seen microscopically at the resection margin, e.g., based on histological assessment of tissues surrounding the tumor resection site). In some embodiments, such a tumor resection site may be characterized by a positive resection margin (i.e., cancer cells are seen microscopically at the resection margin, e.g., based on histological assessment of tissues surrounding the tumor resection site). In some embodiments, such a tumor resection site may be characterized by presence of gross residual tumor antigen. In some embodiments, a target site for administration is or comprises a site in close proximity (e.g., within 4 inches, within 3.5 inches, within 3 inches, within 2.5 inches, within 2 inches, within 1.5 inches, within 1 inches, within 0.5 inches, within 0.4 inches, within 0.3 inches, within 0.2 inches, within 0.1 inches or less; e.g., within 10 centimeters, within 9 centimeters, within 8 centimeters, within 7 centimeters, within 6 centimeters, within 5 centimeters, within 4 centimeters, within 3 centimeters, within 2 centimeters, within 1 centimeter, within 0.5 centimeters or less) to a tumor resection site. In some embodiments, a target site for administration is or comprises a sentinel lymph node. In some embodiments, a target site for administration is or comprises a draining lymph node.

As will be understood by one of ordinary skill in the art, compositions that are useful in accordance with the present disclosure can be administered to a target site in subjects in need thereof using appropriate delivery approaches known in the art. For example, in some embodiments, provided technologies can be amenable for administration by injection. In some embodiments, provided technologies can be amenable for administration by minimally invasive surgery (MIS), e.g., robot-assisted MIS, robotic surgery, and/or laparoscopic surgery, which, for example, typically involve one or more small incisions. In some embodiments, provided technologies can be amenable for administration in the context of accessible and/or cutaneous excisions. In some embodiments, provided technologies can be amenable for administration (e.g., by injection) intraoperatively as part of minimally invasive procedure, e.g., minimally invasive surgery (MIS), e.g., robot-assisted MIS, robotic surgery, and/or laparoscopic surgery, and/or procedure that involves one or more accessible and/or cutaneous excisions. In some embodiments, provided technologies can be amenable for administration (e.g., by injection) involving a robotic surgical system (e.g., a da Vinci System), e.g., in some embodiments for minimally invasive administration. For example, in some embodiments, a composition that may be useful for injection and/or in the context of minimally invasive procedure, e.g., minimally invasive surgery (MIS), e.g., robot-assisted MIS, robotic surgery, and/or laparoscopic surgery and/or procedure that involves one or more accessible and/or cutaneous excisions, is liquid and a polymer combination preparation provided in such a composition is or comprises a polymer solution (e.g., a viscous polymer solution), which upon injection to a target site (e.g., a tumor resection site) in a subject, it transitions from a liquid solution state to a polymer network state (e.g., a hydrogel), which in some embodiments, such a transition is triggered by exposure to the body temperature of the subject. In some embodiments, a polymer combination preparation in a pre-formed polymer network biomaterial that is compressible without adversely impact its structural integrity can be injected, for example, by a minimally invasive procedure, e.g., minimally invasive surgery (MIS), e.g., robot-assisted MIS, robotic surgery, and/or laparoscopic surgery and/or procedure.

In some embodiments, technologies provided herein can be amenable for administration by implantation. For example, in some embodiments, a polymer combination preparation provided in a composition in accordance with the present disclosure is a pre-formed polymer network biomaterial. An exemplary polymer network biomaterial is or comprises a hydrogel. For example, in some embodiments, a provided composition may be administered by surgical implantation to a tumor resection site (e.g., void volume resulting from tumor resection). In some embodiments, a provided composition may be administered by surgical implantation to a tumor resection site and affixed with a bioadhesive. In some embodiments, administration may be performed intraoperatively (i.e., immediately after tumor resection).

In some embodiments, the amount of a polymer combination preparation and/or a therapeutic agent incorporated therein to achieve desirable therapeutic effect(s) such as, e.g., anti-tumor immunity, may vary from subject to subject, depending, for example, on gender, age, and general condition of a subject, type and/or severity of cancer, efficacy of a polymeric biomaterial agonist of innate immunity, and the like.

In some embodiments, the present disclosure provides technologies such that administration of a composition comprising a polymer combination preparation (e.g., ones described herein) by itself and optionally an immunomodulatory payload (e.g., incorporated within a provided polymer combination preparation) is sufficient to provide antitumor immunity and thus does not necessarily require administration of, e.g., a tumor antigen, and/or adoptive transfer of immune cells (e.g., T cells) to a subject in need thereof (e.g., as described herein). Accordingly, in some embodiments, technologies provided herein do not include administering a tumor antigen to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein. In certain embodiments, technologies provided herein do not include adoptive transfer of immune cells (e.g., T cells) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In certain embodiments, the present disclosure provides technologies such that administration of a polymer combination preparation is particularly effective when administered as a co-therapy with e.g., a tumor antigen, and/or adoptive transfer of immune cells (e.g., T cells, NK cells, etc.). In certain embodiments, technologies provided herein include adoptive transfer of immune cells (e.g., T cells, NK cells, etc.) to a subject, e.g., within 1 month or less (including, e.g., within 3 weeks, within 2 weeks, within 1 week, within 5 days, within 3 days, within 1 day, within 12 hours, within 6 hours) after the subject has received a composition as described and/or utilized herein.

In some embodiments, the present disclosure provides technologies such that administration of a polymer combination preparation (e.g., ones described herein comprising HA and/or chitosan as a second polymer) by itself is sufficient to elicit or promote antitumor immunity and thus does not necessarily require administration of an immunomodulatory payload to a subject in need thereof (e.g., as described herein). Accordingly, in some embodiments, technologies provided herein do not include administering an immunomodulatory payload to a subject, e.g., within 1 month or less (including, e.g., within 3 weeks, within 2 weeks, within 1 week, within 5 days, within 3 days, within 1 day, within 12 hours, within 6 hours), after the subject has received a composition as described and/or utilized herein.

In some embodiments, technologies provided herein are useful for treatment of cancer in a subject. In some embodiments, technologies provided herein are for use in treatment of a resectable tumor. In some embodiments, technologies provided herein are for use in treatment of a solid tumor (e.g., but not limited to a blastoma, a carcinoma, a germ cell tumor, and/or a sarcoma). In some embodiments, technologies provided herein are for use in treatment of lymphoma present in a spleen or a tissue outside of a lymphatic system, e.g., a thyroid or stomach.

In some embodiments, technologies provided herein are useful for treating a cancer including, but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi’s sarcoma, multiple idiopathic hemorrhagic sarcoma); endometrial cancer (e.g., uterine cancer, uterine sarcoma); esophageal cancer (e.g., adenocarcinoma of the esophagus, Barrett’s adenocarcinoma); Ewing’s sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget’s disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget’s disease of the vulva), or any combination thereof.

In certain embodiments, the cancer is breast cancer. In certain embodiments, the cancer is skin cancer. In certain embodiments, the cancer is melanoma. In certain embodiments, the cancer is lung cancer. In certain embodiments, the cancer is kidney cancer. In certain embodiments, the cancer is liver cancer. In certain embodiments, the cancer is pancreatic cancer. In certain embodiments, the cancer is colorectal cancer. In certain embodiments, the cancer is bladder cancer. In certain embodiments, the cancer is lymphoma. In certain embodiments, the cancer is prostate cancer. In certain embodiments, the cancer is thyroid cancer. In certain embodiments, the cancer is brain cancer. In certain embodiments, the cancer is stomach cancer. In certain embodiments, the cancer is esophageal cancer.

In some embodiments, technologies provided herein are useful in treating adenocarcinoma, adrenal gland cancer, anal cancer, angiosarcoma, appendix cancer, bile duct cancer, bladder cancer, bone cancer, brain cancer, breast cancer, bronchus cancer, carcinoid tumor, cardiac tumor, cervical cancer, choriocarcinoma, chordoma, colorectal cancer, connective tissue cancer, craniopharyngioma, ductal carcinoma in situ, endotheliosarcoma, endometrial cancer, ependymoma, epithelial carcinoma, esophageal cancer, Ewing’s sarcoma, eye cancer, familiar hypereosinophilia, gall bladder cancer, gastric cancer, gastrointestinal carcinoid tumor, gastrointestinal stromal tumor (GIST), germ cell cancer, head and neck cancer, hemangioblastoma, histiocytosis, Hodgkin lymphoma, hypopharynx cancer, inflammatory myofibroblastic tumors, intraepithelial neoplasms, immunocytic amyloidosis, Kaposi sarcoma, kidney cancer, liver cancer, lung cancer, leiomyosarcoma (LMS), melanoma, midline tract carcinoma, multiple endocrine neoplasia syndrome, muscle cancer, mesothelioma, myeloproliferative disorder (MPD), nasopharynx cancer, neuroblastoma, neurofibroma, neuroendocrine cancer, non-Hodgkin lymphoma, osteosarcoma, ovarian cancer, pancreatic cancer, paraneoplastic syndromes, parathyroid cancer, papillary adenocarcinoma, penile cancer, pharyngeal cancer, pheochromocytoma, pinealoma, pituitary cancer, pleuropulmonary blastoma, primitive neuroectodermal tumor (PNT), plasma cell neoplasia, prostate cancer, rectal cancer, retinoblastoma, rhabdomyosarcoma, salivary gland cancer, sebaceous gland carcinoma, skin cancer, small bowel cancer, small intestine cancer, soft tissue sarcoma, stomach cancer, sweat gland carcinoma, synovioma, testicular cancer, thymic cancer, thyroid cancer, urethral cancer, uterine cancer, vaginal cancer, vascular cancer, vulvar cancer, or a combination thereof.

In some embodiments, a method provided herein may comprise administering to a target site (e.g., as described herein) in a tumor resection subject a provided composition and, optionally, monitoring the tumor resection site or distal sites for risk or incidence of tumor regrowth or tumor outgrowth in the subject after the administration, e.g., every 3 months or longer after the administration, including, e.g., every 6 months, every 9 months, every year, or longer. When the subject is determined to have risk or incidence of tumor recurrence based on the monitoring report, in some embodiments, a subject can be administered with a second composition (e.g., as described herein) and/or a different treatment regimen (e.g., chemotherapy).

In some embodiments, technologies provided herein may be useful for treating subjects who are suffering from metastatic cancer. For example, in some embodiments, a method provided herein may comprise administering to a target site (e.g., as described herein) in a subject suffering from one or more metastases who has undergone a tumor resection (e.g., surgical resection of a primary tumor) and, optionally, monitoring at least one metastatic site in the subject after the administration, e.g., every 3 months or longer after the administration, including, e.g., every 6 months, every 9 months, every year, or longer. Based on results of the monitoring report, in some embodiments, a subject can be administered with a second composition (e.g., as described herein) and/or a different treatment regimen (e.g., chemotherapy).

In certain embodiments, the methods described herein do not comprise administering a provided composition prior to tumor resection. In certain embodiments, the methods described herein do comprise administering a provided composition prior to tumor resection. In certain embodiments, technologies provided herein comprise administering a provided composition to a tumor resection site concurrently to tumor resection. In certain embodiments, technologies provided herein comprise administering a provided composition to a tumor resection site following tumor resection.

It will be also appreciated that compositions described herein can be administered in combination with one or more additional pharmaceutical agents and/or therapeutic regimen. For example, in some embodiments, compositions described herein can be administered as part of a combination therapy. For example, compositions can be administered in combination with additional pharmaceutical agents that reduce and/or modify their metabolism, inhibit their excretion, and/or modify their distribution within the body. In some embodiments, a composition as described herein is administered in conjunction with systemic therapies, such as chemotherapy, radiation therapy, and/or immune modulation therapy. In some embodiments, an immune modulation therapy may include systemic and/or localized administration of agents such as small molecules, peptides, proteins, saccharides, steroids, antibodies, fusion proteins, nucleic acid agents (e.g., but not limited to antisense polynucleotides, ribozymes, and small interfering RNAs), peptidomimetics, and the like. For example, in some embodiments, a combination therapy may comprise a composition as described herein and an immune checkpoint inhibition therapy (e.g., via inhibition of PD-1/PD-L1 pathway). In some embodiments, a combination therapy may comprise a composition as described herein and a chemotherapeutic agent. Suitable chemotherapeutic agents can be found among any of a variety of classes of anti-cancer agents including, but not limited to, alkylating agents, anti-metabolites, topoisomerase inhibitors, and/or mitotic inhibitors. In some embodiments, compositions as described herein are administered as part of a combination therapy prior to, during, and/or after, at least one or more additional therapies. It will also be appreciated that the additional therapy employed may achieve a desired effect for the same disorder, and/or it may achieve different effects. In certain embodiments, an additional pharmaceutical agent is not adoptively transferred cells. In certain embodiments, an additional pharmaceutical agent is not T cells. In certain embodiments, an additional pharmaceutical agent is administered multiple days or weeks after administration of a composition described herein.

In some embodiments, polymer preparations comprising provided herein may be useful to provide sustained release of a payload incorporated herein.

In certain embodiments, technologies provided herein may be useful for treating subjects who are suffering from a wide array of maladies for which localized agent release may be advantageous. In certain embodiments, technologies provided herein may be used in regenerative medicine. In certain embodiments, technologies provided herein may be used in tissue engineering. In certain embodiments, technologies provided herein may be used to aid in medical imaging (e.g., X-ray, CT scanning, and/or radioisotope imaging). In certain embodiments, technologies provided herein may be used in dentistry (e.g., tooth repair). In certain embodiments, technologies provided herein may be used in dermatological applications (e.g., injections to treat facial wrinkles and or folds). In certain embodiments, technologies provided herein may be used in cosmetic and/or plastic surgery. In certain embodiments, technologies provided herein may be used in orthopedic applications (e.g., bone healing, osteoarthritis, spinal fusion, and/or discs). In certain embodiments, technologies provided herein may be used in the treatment of incontinence and other urological indications (e.g., urinary and/or anal). In certain embodiments, technologies provided herein may be used in the treatment of heart failure. In certain embodiments, technologies provided herein may be used in the treatment of hearing loss. In certain embodiments, technologies provided herein may be used in epidermal and/or internal wound dressing. In certain embodiments, technologies provided herein may be used in the prevention of post-operative adhesion. In certain embodiments, technologies provided herein may be used in cancer immunotherapy, including local extended delivery of immunomodulatory molecules. In certain embodiments, technologies provided herein may be used in treatment of autoimmune and/or rheumatic diseases (e.g., through localized and/or extended delivery of immunomodulatory molecules). In certain embodiments, technologies provided herein may be used in treatment of fibrosis and/or scarring (e.g., through localized and/or extended delivery of anti-fibrotic molecules for the prevention or healing of fibrosis and/or scarring). In certain embodiments, technologies provided herein may be used in treatment of infection (e.g., through localized and/or extended delivery of anti-infective molecules for the prevention and/or treatment of infection, e.g., azithromycin, remdesivir and/or any suitable antibiotics and/or antivirals as known in the art). In certain embodiments, technologies provided herein may be used in pain alleviation (e.g., through localized and/or extended delivery of analgesic molecules for the alleviation of pain, e.g., ketorolac, bupivacaine, and/or any suitable analgesic as known in the art).

In certain embodiments, technologies provided herein may be particularly useful for the extended release of molecules for treatment of ocular pathologies. In certain embodiments, provided technologies may be particularly amenable for intravitreal injection. In certain embodiments, provided technologies may be particularly amenable for topical administration. In certain embodiments, provided technologies may be used for treating glaucoma and/or ocular hypertension (e.g., through localized and/or extended release of beta (adrenergic) blockers, prostaglandin analogs, carbonic anhydrase inhibitors, parasympathetic analogs, alpha 2 adrenergic agonists, Rho kinase inhibitors, and/or docosanoids). In certain embodiments, provided technologies may be used for treating age-related macular degeneration (e.g., through localized and/or extended release of any anti-VEGF agent, VEGF inhibitor, anti-VEGFR agent, and/or VEGFR inhibitor as are known in the art). In certain embodiments, provided technologies may be used for treating symptomatic vitreomacular adhesion (e.g., through localized and/or extended release of ocriplasmin and/or any alpha-2 antiplasmin reducer as known in the art). In certain embodiments, provided technologies may be used for treating post-operative inflammation following any ocular surgery (e.g., through localized and/or extended release of ketorolac, loteprednol, dexamethasone, corticosteroids, and/or any suitable anti-inflammatory agent as known in the art). In certain embodiments, provided technologies may be used to deliver anesthetic agents for ophthalmologic procedures (e.g., localized and/or extended delivery of lidocaine and/or any appropriate anesthetic known in the art). In certain embodiments, provided technologies may be used for treating allergic conjunctivitis via either topical or intracanalicular administration (e.g., through local and/or extended delivery of histamine H1 receptor antagonists and/or dexamethasone). In certain embodiments, provided technologies may be used for treating bacterial conjunctivitis and/or corneal ulcers (e.g., localized and/or extended delivery of fluoroquinolone and/or other suitable antibacterial agents as known in the art). In certain embodiments, provided technologies may be used for treating cystinosis (e.g., localized and/or extended delivery of cysteamine hydrochloride and/or other suitable cysteine depleting and/or somatostatin inhibiting agents as known in the art). In certain embodiments, provided technologies may be used for treating neurotrophic keratitis (e.g., localized and/or extended delivery of nerve growth factor and/or other suitable anti-neurotrophic keratitis agents as known in the art). In certain embodiments, provided technologies may be used for treating macular edema following branch or central retinal vein occlusion (e.g., localized and/or extended delivery of dexamethasone and/or other suitable corticosteroid agents as known in the art). In certain embodiments, provided technologies may be used for treating dry eye (e.g., localized and/or extended delivery of cyclosporine and/or other suitable immunomodulatory agents). In certain embodiments, provided technologies may be used for treating HSV-mediated corneal inflammation (e.g., localized and/or extended delivery of trifluridine and/or other suitable antiviral agents as known in the art).

In certain embodiments, a subject being treated is a mammal. In certain embodiments, a subject is a human. In certain embodiments, a subject is a tumor resection human subject. In certain embodiments, a subject is a human subject that is not amenable to tumor resection surgery. In certain embodiments, a subject is a human patient who has received neoadjuvant (pre-operative) therapy. In certain embodiments, a subject is a human patient who has not received neoadjuvant therapy. In certain embodiments, a subject is a human patient who has received neoadjuvant (pre-operative) chemotherapy. In certain embodiments, a subject is a human patient who has not received neoadjuvant (pre-operative) chemotherapy. In certain embodiments, a subject is a human patient who has received neoadjuvant radiation therapy. In certain embodiments, a subject is a human patient who has not received neoadjuvant radiation therapy. In certain embodiments, a subject is a human patient who has received neoadjuvant chemotherapy and radiation therapy. In certain embodiments, a subject is a human patient who has received neoadjuvant molecular targeted therapy. In certain embodiments, a subject is a human patient who has not received neoadjuvant molecular targeted therapy. In certain embodiments, a subject is a human patient who has not received neoadjuvant chemotherapy. In some embodiments, a subject is receiving, has received, or will receive immune checkpoint blockade therapy. In certain embodiments, a subject is receiving immune checkpoint blockade therapy. In certain embodiments, a subject is a human patient who has received and/or is receiving a molecular targeted therapy (e.g., therapies such as those described as neoadjuvants and/or adjuvants) as the sole therapeutic intervention (e.g., a subject for whom surgical resection is not a viable option). In some embodiments, a subject is receiving, has received, or will receive certain other cancer therapeutics (e.g., including but not limited to costimulation, oncolytic virus, CAR T cells, transgenic TCRs, TILs, vaccines, BiTE, ADC, cytokines, modulators of innate immunity, or any combination of these). In certain embodiments, a subject is a human patient who has received neoadjuvant immunotherapy, including immune checkpoint blockade (e.g., anti-CTLA-4, anti-PD-1, and/or anti-PD-L1). In certain embodiments, a subject is a human patient who has not received and/or will not receive neoadjuvant immunotherapy, including immune checkpoint blockade (e.g., anti-CTLA-4, anti-PD-1, and/or anti-PD-L1). In certain embodiments, a subject is a human patient whose tumor has not objectively responded and/or will not objectively respond to neoadjuvant therapy (as defined by Response Evaluation Criteria in Solid Tumors (RECIST) or immune-related Response Criteria (irRC)) (e.g., stable disease, progressive disease). In certain embodiments, a subject is a human patient whose target lesion has objectively responded and/or is objectively responding to neoadjuvant therapy (e.g., partial response, complete response). Non-target lesions may exhibit an incomplete response, stable disease, or progressive disease. In certain embodiments, a subject is a human patient who would be eligible to receive immunotherapy in an adjuvant (post-operative) setting. In certain embodiments, a subject is a domesticated animal, such as a dog, cat, cow, pig, horse, sheep, or goat. In certain embodiments, a subject is a companion animal such as a dog or cat. In certain embodiments, a subject is a livestock animal such as a cow, pig, horse, sheep, or goat. In certain embodiments, a subject is a zoo animal. In another embodiment, a subject is a research animal, such as a rodent, pig, dog, or non-human primate. In certain embodiments, a subject is a non-human transgenic animal such as a transgenic mouse or transgenic pig.

### VII. Kits

The present disclosure also provides kits that find use in practicing technologies as provided herein. In some embodiments, a kit comprises a composition or a pharmaceutical composition described herein and a container (e.g., a vial, ampule, bottle, syringe, and/or dispenser package, or other suitable container). In some embodiments, a kit comprises delivery technologies such as syringes, bags, etc., or components thereof, which may be provided as a single and/or multiple use item. In some embodiments, one or more component(s) of a composition or a pharmaceutical composition described herein are separately provided in one or more containers. For example, individual components of a polymer combination preparation (e.g., ones described herein, for example, but not limited to poloxamer and a second polymer such as hyaluronic acid and/or a chitosan or variants thereof) may be, in some embodiments, provided in separate containers. In some such embodiments, individual components of a biomaterial (e.g., ones described herein, for example, but not limited to poloxamer and a second polymer such as hyaluronic acid and/or a chitosan or variants thereof) may be each provided independently as dry lyophilized powder, dry particles, or a liquid. In some embodiments, individual components of a polymer combination preparation (e.g., ones described herein, for example, but not limited to poloxamer and a second polymer such as hyaluronic acid and/or a chitosan or variants thereof) may be provided as a single mixture in a container. In some such embodiments, a single mixture may be provided as dry lyophilized powder, dry particles, or a liquid (e.g., a homogenous liquid).

In some embodiments, a polymer combination preparation (e.g., ones described herein) may be provided as a pre-formed polymer network biomaterial in a container. In some embodiments, such a pre-formed polymer network biomaterial (e.g., a hydrogel) may be provided in a dried state. In some embodiments, a pre-formed polymer network biomaterial (in a form of a viscous polymer solution) may be provided in a container.

In some embodiments, provided kits may optionally include a container comprising a pharmaceutical excipient for dilution or suspension of a composition or pharmaceutical composition described herein. In some embodiments, provided kits may include a container comprising an aqueous solution. In some embodiments, provided kits may include a container comprising a buffered solution.

In some embodiments, provided kits may comprise a payload such as a therapeutic agent described herein. For example, in some embodiments, a payload may be provided in a separate container such that it can be added to a polymer combination preparation liquid mixture (e.g., as described herein) prior to administration to a subject. In some embodiments, a payload may be incorporated in a polymer combination preparation described herein.

In certain embodiments, provided kits may not comprise an immunomodulatory payload. For example, in some embodiments, provided kits may not comprise an activator of innate immune response. In some embodiments, provided kits may not comprise an activator of adaptive immune response. In some embodiments, provided kits may not comprise an inhibitor of a proinflammatory response. In some embodiments, provided kit may not comprise an immunomodulatory cytokine.

In certain embodiments, a kit described herein further includes instructions for practicing methods described herein. A kit described herein may also include information as required by a regulatory agency such as the U.S. Food and Drug Administration (FDA). In certain embodiments, information included in kits provided herein is prescribing information, e.g., for treatment for cancer. Instructions may be present in kits in a variety of forms, one or more of which may be present in the kits. One form in which these instructions may be present is as printed information on a suitable medium or substrate, e.g., a piece or pieces of paper on which the information is printed, in the packaging of kits, in a package insert, etc. Yet another means may be a computer readable medium, e.g., diskette, CD, USB drive, etc., on which instructional information has been recorded. Yet another means that may be present is a website address which may be used via the internet to access instructional information. Any convenient means may be present in the kits.

Other features of the invention will become apparent in the course of the following description of exemplary embodiments, which are given for illustration of the invention and are not intended to be limiting thereof.

### EXEMPLIFICATION

In order that the embodiments described herein may be more fully understood, the following examples are set forth. It should be understood that these examples are for illustrative purposes only and are not to be construed as limiting the present disclosure in any manner.

### Example 1. Exemplary Materials and Methods for Preparation and Characterization of Exemplary Polymer Combination Preparations Described Herein and Reference Polymer Biomaterials

The present example relates to the preparation and characterization of exemplary polymer combinations as described herein. In some embodiments, a generality may be observed wherein as the concentration of one biomaterial increases (e.g., poloxamer), the concentration of the at least one additional biomaterial (e.g., hyaluronic acid and/or chitosan/modified chitosan) required to make a suitable polymer network trends towards a decreasing value. In some embodiments, this generality applies in the opposite direction (e.g., suitable polymer networks formed using lower poloxamer concentrations may use higher concentrations of the at least one additional biomaterial).

Exemplary polymer combination preparations comprising poloxamer and hyaluronic acid are shown below:

Preparation comprising 13.5% (w/w) Poloxamer 407 and 0.65% (w/w) 1.5 MDa hyaluronic acid in 0.1 M NaHCO₃, 0.9% saline pH 8.1 or 25 mM phosphate buffer pH 7.4 or pH 8.

Preparation comprising 10-12.5% (w/w) Poloxamer 407 and 0.65-1% (w/w) 1.5 MDa hyaluronic acid in 0.1 M NaHCO₃, 0.9% saline pH 8.1 or 25 mM phosphate buffer pH 7.4 or pH 8.

Preparation comprising 9-10% (w/w) Poloxamer 407 and 1-1.2% (e.g., 1.1%) (w/w) 1.5 MDa hyaluronic acid in 25 mM phosphate buffer pH 7.4 or pH 8.

Preparation comprising 8-9% (w/w) Poloxamer 407 and 1.65-1.75% (w/w) 1.32 MDa hyaluronic acid in 25 mM phosphate buffer pH 7.4 or pH 8.

Preparation comprising 10% (w/w) Poloxamer 407 and 1-1.5% (e.g.,1.3%) (w/w) 773 kDa hyaluronic acid in 10 mM PBS pH 7.4 or 25 mM phosphate buffer pH 7.4 or pH 8.

Preparation comprising 9-10% (w/w) Poloxamer 407 and 1.2-2.5% (w/w) 730 kDa hyaluronic acid in 10 mM PBS pH 7.4 or 25 mM phosphate buffer pH 7.4 or pH 8.

Preparation comprising 9-10% (w/w) Poloxamer 407 and 1.2-2.5% (w/w) 730 kDa hyaluronic acid in 10 mM PBS pH 8 or 25 mM phosphate buffer pH 8.

Preparation comprising 9-11.5% (w/w) Poloxamer 407 and 2-2.75% (w/w) 730 kDa hyaluronic acid in 10 mM PBS pH 7.4 or 25 mM phosphate buffer pH 7.4 or pH 8.

Preparation comprising 12.3% (w/w) Poloxamer 407 and 1.625% (w/w) 730 kDa hyaluronic acid in 25 mM phosphate buffer pH 7.4 or pH 8.

Preparation comprising 8% (w/w) Poloxamer 407 and 1.75%-2.25% (w/w) 337 kDa hyaluronic acid in 25 mM phosphate buffer pH 7.4 or pH 8.

Preparation comprising 10% (w/w) Poloxamer 407 and 2-6% (w/w) 309 kDa hyaluronic acid in 25 mM phosphate buffer pH 7.4 or pH 8.

Preparation comprising 8-12.5% (w/w) Poloxamer 407 and 1-4% (w/w) 119 or 120 kDa hyaluronic acid in 25 mM phosphate buffer pH 7.4 or pH 8.

Preparation comprising 10% (w/w) Poloxamer 407 and 2-6% (w/w) 119 or 120 kDa hyaluronic acid in 25 mM phosphate buffer pH 7.4 or pH 8.

Preparation comprising 8-12.5% (w/w) Poloxamer 407 and 1-4% (w/w) 187 kDa hyaluronic acid in 25 mM phosphate buffer pH 7.4 or pH 8.

Preparation comprising 10% (w/w) Poloxamer 407 and 2-6% (w/w) 187 kDa hyaluronic acid in 25 mM phosphate buffer pH 7.4 or pH 8.

Preparation comprising 8-10% (w/w) Poloxamer 338 and 1-1.5% (w/w) 1.32 MDa hyaluronic acid in 25 mM phosphate buffer pH 7.4 or pH 8.

Preparation comprising 8-10% (w/w) Poloxamer 338 and 1.4-2% (w/w) 730 kDa hyaluronic acid in 25 mM phosphate buffer pH 7.4 or pH 8.

Preparation comprising 8-10% (w/w) Poloxamer 338 and 1.75-2.5% (w/w) 119 kDa hyaluronic acid in 25 mM phosphate buffer pH 7.4 or pH 8.

Exemplary polymer combination preparations comprising poloxamer and chitosan or modified chitosan are shown below:

Preparation comprising 13.5% (w/w) Poloxamer 407 and 0.65-1.3% (w/w) carboxymethyl chitosan in 10 mM PBS, 33 mM NaHCO₃, 0.45% saline pH 8.1, or 25 mM phosphate buffer pH7.4.

Preparation comprising 8-12.5% (w/w) Poloxamer 407 and 2.5-5% (w/w) carboxymethyl chitosan in 10 mM PBS, 33 mM NaHCO₃, 0.45% saline pH 8.1, or 25 mM phosphate buffer pH7.4.

Exemplary polymer combination preparations comprising poloxamer, hyaluronic acid, and chitosan or modified chitosan are shown below:

Preparation comprising 8-12.5% (w/w) Poloxamer 407, 2-6% (w/w) 119 kDa hyaluronic acid, and 0.2-5% (w/w) carboxymethyl chitosan in 25 mM phosphate buffer pH7.4.

Preparation comprising 8-12.5% (w/w) Poloxamer 407, 2-6% (w/w) 187 kDa hyaluronic acid, and 0.2-5% (w/w) carboxymethyl chitosan in 25 mM phosphate buffer pH7.4.

Preparation comprising 8-12.5% (w/w) Poloxamer 407, 1-3% (w/w) 773 kDa hyaluronic acid, and 0.1-1% (w/w) carboxymethyl chitosan in 25 mM phosphate buffer pH7.4.

Preparation comprising 8-12.5% (w/w) Poloxamer 407, 1.0-3% (w/w) 730 kDa hyaluronic acid, and 0.1-1% (w/w) carboxymethyl chitosan in 25 mM phosphate buffer pH7.4.

Preparation comprising 6-10% (w/w) Poloxamer 407, 1.25-5% (w/w) 309 kDa hyaluronic acid, and 0.2-1.5% (w/w) carboxymethyl chitosan in 25 mM phosphate buffer pH7.4.

Preparation comprising 6-10% (w/w) Poloxamer 407, 1.25-5% (w/w) 119 kDa hyaluronic acid, and 0.5-2.5% (w/w) carboxymethyl chitosan in 25 mM phosphate buffer pH7.4.

Preparation comprising 8-12.5% (w/w) Poloxamer 407, 1.25-5% (w/w) 119 kDa hyaluronic acid, and 0.2-2% (w/w) carboxymethyl chitosan in 25 mM phosphate buffer pH7.4. Rheological analysis of exemplary polymer combination preparations:

Rheological analysis of hydrogels formed from polymer combination preparations was performed using a TA instruments Discovery HR2 rheometer using a 20 mm parallel plate, a 1,500 µm gap, and a frequency sweep of 0.1 Hz to 10 Hz, 0.4% strain at 37° C., soak time of 120 s and run time of 60 s. Maximum storage modulus (Pa) and minimum phase angle δ° were measured.

### Cell Line and Cell Culture:

4T1-Luc2 breast cancer cells were cultured in RPMI-1640 medium, with 10% fetal bovine serum (FBS) and 1% penicillin/streptomycin. All cells were cultured in a 37° C. in a humidified incubator, with 5% CO₂.

### Mouse Tumor Models:

All animal experiments were performed using 6-8 weeks old female BALB/c mice (Jackson Laboratories, #000651). For animal survival studies, 10⁵ 4T1-Luc2 cells were inoculated orthotopically into the fourth mammary fat pad of a mouse. Mice were size-matched and randomly assigned to treatment groups, and surgery was performed on day 10 or day 12 after tumor inoculation. Tumor sizes were measured with calipers. For primary tumor resection, mice were anesthetized with 2% isoflurane, the tumor was resected, and a hydrogel was placed in the surgical site at the time of surgery.

### Exemplary Methods for Hydrogel Preparation:

### (I) Chemically Crosslinked Hyaluronic Acid (HA) Hydrogels:

HyStem HA hydrogels were prepared using the HyStem hydrogel kit (ESI Bio, GS1004). First, 120 µL of Glycosil was added into a Teflon mold (9 mm diameter). Next, 10 µL of an immunomodulatory payload was optionally added and stirred to create a homogeneous mixture. Finally, 30 µL of Extralink was added, and the hydrogel was left to crosslink for one hour.

### (II) Poloxamer-HA Hydrogels:

Poloxamer-HA hydrogels were prepared by combining appropriate amount of poloxamer (e.g., a solid particle preparation or a liquid preparation) and a solid particle (e.g., powder) preparation of HA in a 4 mL vial (optionally along with 5 µL of an immunomodulatory payload (e.g. R848 (Sigma #SML0196) prepared at 40 mg/mL in DMSO)) to prepare a solution mixture and mixing the solution mixture at 300 rpm for 15 min and then at 100 rpm for overnight. To induce gel formation, the solution mixture was placed in a water bath at 37° C. After 10-15 minutes at 37° C., the sample was observed for gel formation or phase separation (no gel formation). The resulting gels were then subjected to rheological analysis, e.g., as described herein.

In some embodiments, the solution mixture after overnight mixing was then cooled in ice for at least 10 min before transferring 200 µL to a 1 mL syringe (BD-309602) for in vivo administration experiment.

### (III) Poloxamer-CMCH Hydrogels:

Poloxamer-CMCH hydrogels were prepared by weighing appropriate amount of poloxamer and CMCH in an appropriate buffer in a 20 mL vial to prepare a solution mixture and mixing the solution mixture at 300 rpm for 15 min and then at 100 rpm overnight. To induce gel formation, the solution mixture was placed in a water bath at 37° C. After 10-15 minutes at 37° C., the sample was observed for gel formation or phase separation (no gel formation). The resulting gels were then subjected to rheological analysis, e.g., as described herein.

In some embodiments, the solution mixture after overnight mixing was then cooled in ice for at least 10 min before transferring 200 µL to a 1 mL syringe (BD-309602) for in vivo administration experiment.

### In Vitro Release Study:

In some embodiments, to determine the release kinetics of each payload from a test polymer combination preparation hydrogel, 0.15 mL of a Hystem hydrogel or a polymer combination preparation (e.g., as described herein) was loaded with a payload (e.g., a lipophilic or hydrophilic agent) and plated into a 96 well-plate. The formulations were heated at 37° C. for 30 minutes. Then, 0.15 mL of pre-warmed 37° C. release buffer (e.g., water for Hystem or pH 7.4 phosphate buffered saline (PBS) for the polymer combination preparations) was added to each well. At the specified time points, 0.1 mL of sample was removed from each well, the absorbance was measured, and concentration was determined using a calibration curve.

### Example 2. Gelation Properties of Exemplary Polymer Combination Preparations

The present Example 2 describes gelation properties of certain test polymer combination preparations comprising Poloxamer 407 and a second polymer component, which may be or comprise a carbohydrate polymer (e.g., hyaluronic acid and/or chitosan or a variant thereof).

In many embodiments, polymer combination preparations as described and/or utilized herein are temperature-responsive such that it transition from a precursor state (e.g., a polymer solution or colloid) to a polymer network state in response to a temperature change. In some embodiments, a polymer network state is a more viscous liquid or colloid than the precursor state. In some embodiments, a polymer network state is a hydrogel.

In some embodiments, temperature-responsive polymer combination preparations as described and/or utilized herein transition from a precursor state to a polymer network state at a gelation temperature (e.g., a temperature that is or above the critical gelation temperature of the polymer combination preparation) in the absence of any chemical crosslinkers. In some embodiments, a gelation temperature may be a temperature of 35-39° C. (e.g., at a temperature of 37° C.). In some embodiments, temperature-responsive polymer combination preparations as described and/or utilized herein transition from a precursor state to a polymer network state at the body temperature of a subject (e.g., a human subject) in the absence of any chemical crosslinkers. In some embodiments, temperature-responsive polymer combination preparations as described and/or utilized herein exhibit a sol-gel transition temperature of approximately 28-35° C. or of approximately 20-28° C.

In some embodiments, polymer combination preparations, and/or individual components thereof were prepared in a suitable buffer. In certain embodiments, polymer combination preparations, and/or individual components thereof were prepared in an aqueous buffer system. Examples of suitable aqueous buffer systems at an appropriate pH include, e.g., but are not limited to phosphate buffer and/or bicarbonate buffer at an appropriate pH. In some embodiments, polymer combination preparations, and/or individual components thereof were prepared in phosphate-buffered saline (PBS), sodium phosphate saline (SPS), potassium dihydrogen phosphate buffer, dipotassium hydrogen phosphate buffer, sodium bicarbonate buffer, sodium citrate buffer, sodium acetate buffer, TRIS buffer, and/or HEPES buffer, each at an appropriate pH. In some embodiments, polymer combination preparations, and/or individual components thereof were prepared in an aqueous buffer system at a concentration range of from 1 mM to 500 mM, or from 5 mM to 250 mM, or from 10 mM to 150 mM. In certain embodiments, a suitable aqueous buffer (e.g., a phosphate buffer) was prepared at a concentration of 10 mM -50 mM. In certain embodiments, a suitable aqueous buffer (e.g., a bicarbonate buffer) was prepared at a concentration of 100-200 mM.

In some embodiments, polymer combination preparations, and/or individual components thereof were prepared in a suitable buffer (e.g., ones described herein) with pH around neutral pH. For example, in certain embodiments, polymer combination preparations, and/or individual components thereof can be prepared in a suitable buffer with pH 6-9. In some embodiments, polymer combination preparations, and/or individual components thereof can be prepared in a suitable buffer with pH 7-8. In some embodiments, polymer combination preparations, and/or individual components thereof can be prepared in a suitable buffer with pH 7.2-7.6. In some embodiments, polymer combination preparations, and/or individual components thereof can be prepared in a suitable buffer with pH 7.4. In some embodiments, polymer combination preparations, and/or individual components thereof can be prepared in a suitable buffer with pH 8.0.

To assess gelation properties of various polymer combination preparations, a polymer preparation comprises a poloxamer at a concentration of 12% (w/w) or lower and a second polymer component that is not a poloxamer, was exposed to a target temperature for inducing gelation process (e.g., the body temperature of a subject such as a temperature of 37° C.) for a period of time (e.g., about 15-20 minutes) and then the physical state (e.g., solution vs. gel) of the polymer preparation was observed. Qualitative observations were made to determine initial gel formation characteristics. Polymer combination preparations were considered to have formed a “good gel” when the sample becomes translucent or opaque and does not flow when angled or inverted. The sample maintains the shape of the vessel/vial until temperature drops below the CGT. Relatively “weak gels” were qualitatively determined to have more flow when angled or inverted when compared to “good gels” and less flow when compared to solutions below the respective CGT. For polymer preparations that form hydrogels after exposure to the target gelation temperature, rheological analysis was performed, e.g., to determine storage modulus and/or phase angle of the resulting hydrogels.

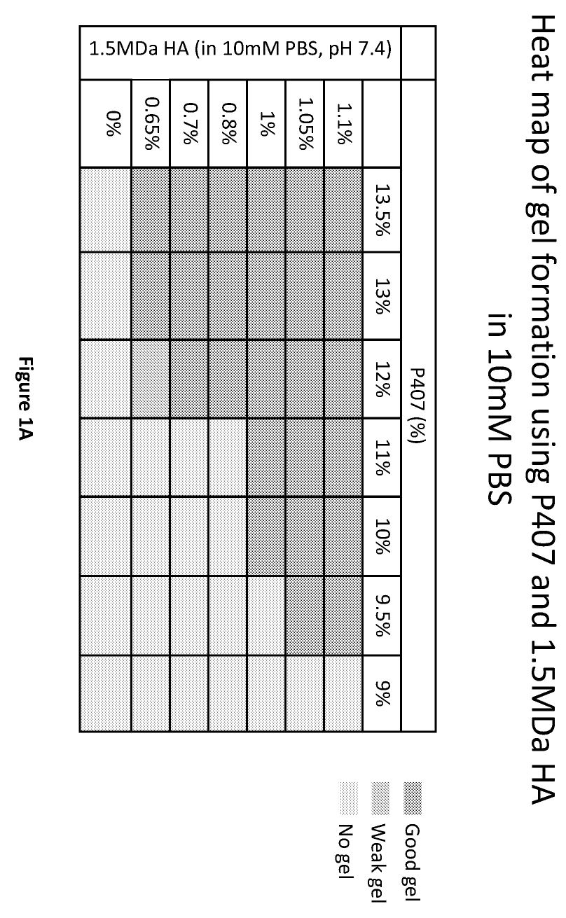

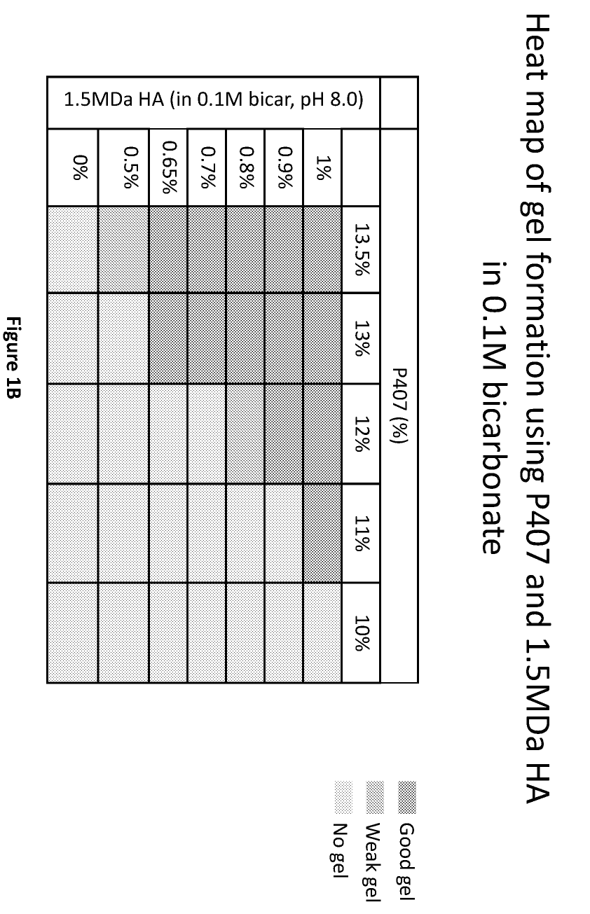

As shown in FIGS. 1A-3, various polymer combination preparations comprising Poloxamer 407 (P407) at 9%-13.5% (w/w) and a carbohydrate polymer (e.g., hyaluronic acid or chitosan or modified chitosan) at 0%-2% (w/w) in a phosphate buffer or a bicarbonate buffer (e.g., pH 7-8) were assessed for their gelation properties.

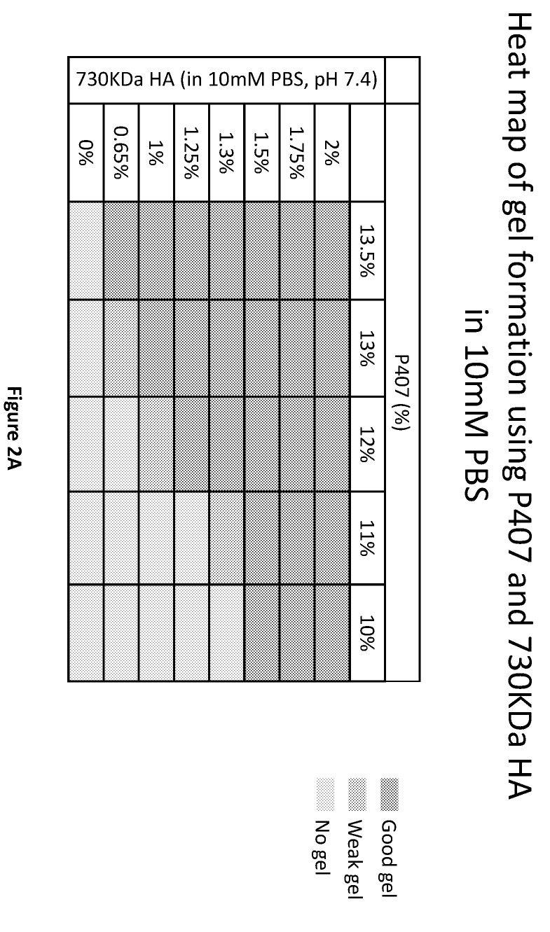

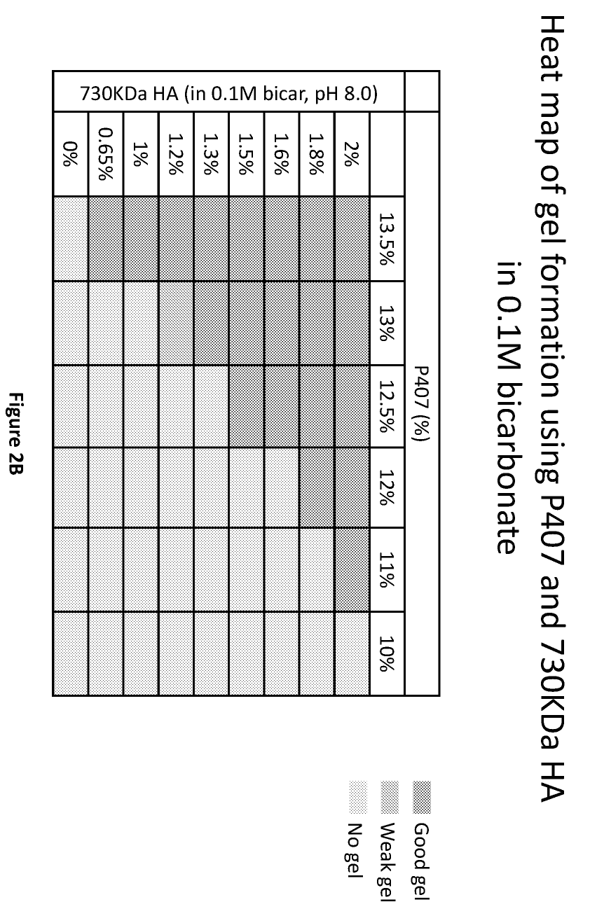

In some embodiments, a carbohydrate polymer included in certain polymer combination preparations is or comprises hyaluronic acid, e.g., having an average molecular weight of 500 kDa- 1.5 MDa. In some embodiments, a carbohydrate polymer included in certain polymer combination preparations is or comprises hyaluronic acid having an average molecular weight of 750 kDa. In some embodiments, a carbohydrate polymer included in certain polymer combination preparations is or comprises hyaluronic acid having an average molecular weight of 1.5 MDa. FIG. 1A shows gel formation from certain polymer combination preparations comprising P407 at a concentration of 9.5%-13.5% (w/w) and 1.5 MDa hyaluronic acid at a concentration of 0.65%-1.1% (w/w) in 10 mM PBS, pH 7.4. FIG. 1B shows gel formation from certain polymer combination preparations comprising P407 at a concentration of 11%-13.5% (w/w) and 1.5 MDa hyaluronic acid at a concentration of 0.5%-1% (w/w) in 0.1 M bicarbonate buffer, pH 8. FIG. 2A shows gel formation from certain polymer combination preparations comprising P407 at a concentration of 10%-13.5% (w/w) and 750 kDa hyaluronic acid at a concentration of 0.65%-2% (w/w) in 10 mM PBS, pH 7.4. FIG. 2B shows gel formation from certain polymer combination preparations comprising P407 at a concentration of 11%-13.5% (w/w) and 730 kDa hyaluronic acid at a concentration of 0.65%-2% (w/w) in 0.1 M bicarbonate buffer, pH 8.

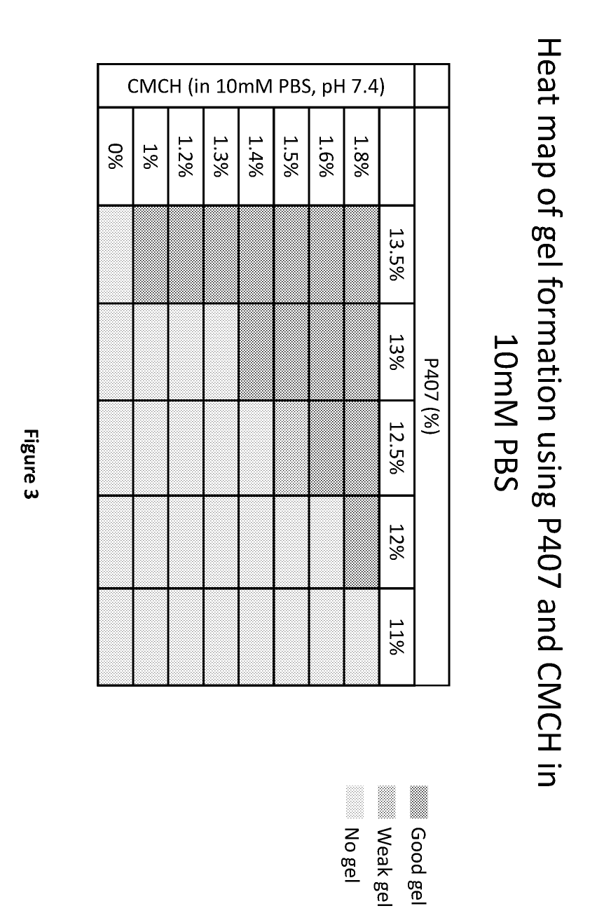

In some embodiments, a carbohydrate polymer included in certain polymer combination preparations is or comprises a modified chitosan (e.g., carboxymethyl chitosan; CMCH). FIG. 3 shows gel formation from certain polymer combination preparations comprising P407 at a concentration of 11%-13.5% (w/w) and CMCH at a concentration of 1%-1.8% (w/w) in 10 mM PBS, pH7.4.

In some embodiments, a carbohydrate polymer included in certain polymer combination preparations is or comprises hyaluronic acid, e.g., having an average molecular weight of 100-900 kDa. In some embodiments, a carbohydrate polymer included in certain polymer combination preparations is or comprises hyaluronic acid having an average molecular weight of about 119 kDa, 187 kDa, 309 kDa, 730 kDa, 773 kDa, 886 kDa or any combination thereof. In some embodiments, such polymer combination preparations as described herein may optionally include modified chitosan.

In some embodiments, a biomaterial polymer combination (e.g., that is a gel at 37° C.) described herein comprises 10% (w/w) poloxamer 407, and 1-2.5% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-4% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 9% (w/w) poloxamer 407, and 3-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 10% (w/w) poloxamer 407, and 2% (w/w) Hyaluronic Acid with a molecular weight of approximately 309 kDa, and optionally 0.1-1% (w/w) modified chitosan.

In some embodiments, a biomaterial polymer combination (e.g., that is a gel at 37° C.) described herein comprises 10% (w/w) poloxamer 407, and 3-4% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 10% (w/w) poloxamer 407, and 3-7% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 9% (w/w) poloxamer 407, and 3-7% (w/w) Hyaluronic Acid with a molecular weight of approximately 100-200 kDa, and optionally 0.1-2.5% (w/w) modified chitosan.

In some embodiments, a biomaterial polymer combination (e.g., that is a gel at 37° C.) described herein comprises 8% (w/w) poloxamer 407, and 2.5-5% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 8% (w/w) poloxamer 407, and 1.5-2.5% (w/w) Hyaluronic Acid with a molecular weight of approximately 309 kDa, and 1-1.5% (w/w) modified chitosan.

In some embodiments, a biomaterial polymer combination (e.g., that is a gel at 37° C.) described herein comprises 8% (w/w) poloxamer 407, and 1.5% (w/w) Hyaluronic Acid with a molecular weight of approximately 773 kDa, and 0.5-1.0% (w/w) modified chitosan.

In some embodiments, a biomaterial polymer combination (e.g., that is a gel at 37° C.) described herein comprises 11-12% (w/w) poloxamer 407, and 3-5% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 1.5-3% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 5-7% (w/w) Hyaluronic Acid with a molecular weight of approximately 100-200 kDa, and optionally 0.1-1% (w/w) modified chitosan.

In some embodiments, a biomaterial polymer combination (e.g., that is a gel at 37° C.) described herein comprises 6-8% (w/w) poloxamer 407, and 2-3% (w/w) Hyaluronic Acid with a molecular weight of approximately 700-800 kDa, and optionally 0.1-1% (w/w) modified chitosan.

In some embodiments, a biomaterial polymer combination (e.g., that is a gel at 37° C.) described herein comprises 9-11% (w/w) poloxamer 407, and 1-2% (w/w) Hyaluronic Acid with a molecular weight of approximately 700-800 kDa, and optionally 0.1-1% (w/w) modified chitosan.

In some embodiments, a biomaterial polymer included in certain polymer combination preparations is or comprises combinations as represented in Table 1, Table 2, Table 3, and Table 4.

TABLE 1

Gelation Properties of Certain Biomaterial Combinations

| MW HA kDa | wt% HA | wt% P407 | wt% CMCH | Gel at 37° C. |
|---|---|---|---|---|
| - | - | 8 | 0.4-2.5 | No |
| - | - | 9 | 0.4-2.5 | No |
| 119 | 2.5-5 | 8 | - | Yes |
| 119 | 3 | 8 | 0.4-0.5 | No |
| 119 | 4-5 | 8 | 0.4-0.5 | Yes |
| 119 | 3-7 | 9 | - | Yes |
| 119 | 3 | 9 | 0.4-0.5 | No |
| 119 | 3-4 | 10 | - | Yes |
| 119 | 3-4 | 10 | 0.5-2.5 | Yes |
| 119 | 3-6 | 11 | - | Yes |
| 119 | 3-4 | 11.5 | - | Yes |
| 187 | 3-4 | 8 | - | Yes |
| 187 | 3-4.1 | 10 | - | Yes |
| 309 | 1.5-2.5 | 8 | - | Yes |
| 309 | 1.5-2.5 | 8 | 1-1.5 | Yes |
| 309 | 2-3 | 10 | - | Yes |
| 730 | 2.7 | 6 | - | Yes |
| 730 | 2.5 | 7 | - | Yes |
| 730 | 2.3 | 8 | - | Yes |
| 730 | 2.2-2.5 | 9 | - | Yes |
| 730 | 1.2-3 | 10 | - | Yes |
| 730 | 1.8-2 | 11 | - | Yes |
| 773 | 2.7 | 6 | - | Yes |
| 773 | 2.5 | 7 | - | Yes |
| 773 | 1.5-2.3 | 8 | - | Yes |
| 773 | 1.2-2 | 10 | - | Yes |
| 773 | 1.3 | 10 | 0.5 | Yes |
| 886 | 1.8 | 9 | - | Yes |
| 886 | 1.5 | 11 | - | Yes |

Formulations comprising polymer combinations as described in Table 2, Table 3, and Table 4 were tested for gelation characteristics and were found to form gels at 37° C. In some embodiments, a polymer combination preparation described herein was considered as forming a gel when the polymer combination preparation changed from a transparent solution to an opaque composition, when the composition was observed to have no flow, and/or when a magnetic stir bar present in the polymer combination preparation did not move in the presence of a magnetic field.

TABLE 2

Compositions comprising low MW HA, demonstrated to form a gel at 37° C.

| wt% P407 | wt% HA | Mw HA kDa | wt% CMCH | wt% P407 | wt% HA | Mw HA kDa | wt% CMCH |
|---|---|---|---|---|---|---|---|
| 10 | 5 | 73 | - | 10 | 3 | 187 | 0.2 |
| 10 | 7.5 | 73 | - | 10 | 3 | 187 | 0.3 |
| 6 | 9 | 119 | - | 10 | 3 | 187 | 2.5 |
| 8 | 2.5 | 119 | - | 10 | 4 | 187 | 0.2 |
| 8 | 3 | 119 | - | 10 | 4 | 187 | 0.3 |
| 8 | 3.7 | 119 | - | 6 | 2.8 | 309 | - |
| 9 | 6 | 119 | - | 8 | 2.25 | 309 | - |
| 9 | 6.5 | 119 | - | 8 | 2.6 | 309 | - |
| 9 | 7 | 119 | - | 8 | 2.7 | 309 | - |
| 10 | 1.95 | 119 | - | 8 | 2.8 | 309 | - |
| 10 | 2 | 119 | - | 9 | 2.5 | 309 | - |
| 10 | 3 | 119 | - | 9 | 2.6 | 309 | - |
| 10 | 4 | 119 | - | 9 | 2.7 | 309 | - |
| 10 | 4.1 | 119 | - | 9 | 2.8 | 309 | - |
| 11 | 1.75 | 119 | - | 10 | 1.5 | 309 | - |
| 11 | 5 | 119 | - | 10 | 2 | 309 | - |
| 11 | 5.5 | 119 | - | 10 | 2.05 | 309 | - |
| 11 | 6 | 119 | - | 10 | 2.2 | 309 | - |
| 11.5 | 3 | 119 | - | 10 | 2.3 | 309 | - |
| 11.5 | 4 | 119 | - | 10 | 2.4 | 309 | - |
| 12 | 1.15 | 119 | - | 10 | 2.5 | 309 | - |
| 8 | 2.5 | 119 | 1.5 | 10 | 2.56 | 309 | - |
| 8 | 4 | 119 | 0.3 | 10 | 2.6 | 309 | - |
| 8 | 4 | 119 | 0.4 | 10 | 2.7 | 309 | - |
| 8 | 5 | 119 | 0.3 | 10 | 2.8 | 309 | - |
| 8 | 5 | 119 | 0.4 | 10 | 3 | 309 | - |
| 10 | 3 | 119 | 0.15 | 10.4 | 2.4 | 309 | - |
| 10 | 3 | 119 | 0.2 | 11 | 1 | 309 | - |
| 10 | 3 | 119 | 0.3 | 11 | 2.4 | 309 | - |
| 10 | 3 | 119 | 0.4 | 11 | 2.5 | 309 | - |
| 10 | 3 | 119 | 0.5 | 11 | 2.6 | 309 | - |
| 10 | 3 | 119 | 1 | 11 | 2.8 | 309 | - |
| 10 | 3 | 119 | 1.5 | 11 | 3.3 | 309 | - |
| 10 | 3 | 119 | 2 | 11 | 3.5 | 309 | - |
| 10 | 3 | 119 | 2.5 | 11.05 | 2.4 | 309 | - |
| 10 | 4 | 119 | 0.3 | 11.2 | 2.2 | 309 | - |
| 10 | 4 | 119 | 0.4 | 11.25 | 2.2 | 309 | - |
| 10 | 4 | 119 | 0.5 | 11.25 | 2.3 | 309 | - |
| 10 | 4 | 119 | 1 | 11.25 | 2.4 | 309 | - |
| 10 | 4 | 119 | 1.5 | 11.25 | 2.5 | 309 | - |
| 10 | 4 | 119 | 5 | 11.5 | 2 | 309 | - |
| 9 | 6.5 | 119/187 | - | 12 | 0.7 | 309 | - |
| 11 | 5.5 | 119/187 | - | 8 | 2 | 337 | - |
| 11.25 | 2.8 | 144 | - | 8 | 2.5 | 337 | - |
| 11.5 | 2.4 | 144 | - | 10 | 1.75 | 337 | - |
| 11.5 | 2.5 | 114 | - | 11 | 2.4 | 309 | - |
| 11.5 | 2.6 | 144 | - | 11 | 2.5 | 309 | - |
| 11.5 | 2.7 | 144 | - | 11 | 2.6 | 309 | - |
| 11.5 | 2.8 | 144 | - | 11 | 2.8 | 309 | - |
| 8 | 3 | 187 | - | 11 | 3.3 | 309 | - |
| 8 | 4 | 187 | - | 11 | 3.5 | 309 | - |
| 9 | 5.5 | 187 | - | 11.05 | 2.4 | 309 | - |
| 10 | 3 | 187 | - | 11.2 | 2.2 | 309 | - |
| 10 | 3.08 | 187 | - | 11.25 | 2.2 | 309 | - |
| 10 | 4 | 187 | - | 11.25 | 2.3 | 309 | - |
| 10 | 4.1 | 187 | - | 11.25 | 2.4 | 309 | - |
| 11 | 4.5 | 187 | - | 11.25 | 2.5 | 309 | - |
| 11.25 | 2.8 | 187 | - | 11.5 | 2 | 309 | - |
| 11.25 | 3 | 187 | - | 12 | 0.7 | 309 | - |

TABLE 3

Compositions comprising high MW HA - demonstrated to form a gel at 37° C.

| wt% P407 | wt% HA | Mw HA kDa | wt% P407 | wt% HA | Mw HA kDa |
|---|---|---|---|---|---|
| 8 | 1.6 | 730 | 11 | 1.2 | 766 |
| 8 | 2 | 730 | 11 | 1.5 | 766 |
| 8 | 2.25 | 730 | 11 | 1.6 | 766 |
| 8.5 | 1.6 | 730 | 11 | 1.7 | 766 |
| 9 | 1.6 | 730 | 11 | 1.8 | 766 |
| 9 | 2.2 | 730 | 11.25 | 1.8 | 766 |
| 9.5 | 1.6 | 730 | 12 | 0.7 | 766 |
| 10 | 1.2 | 730 | 6 | 2.4 | 773 |
| 10 | 1.3 | 730 | 6 | 2.7 | 773 |
| 10 | 1.4 | 730 | 7 | 2.5 | 773 |
| 10 | 1.5 | 730 | 8 | 2.3 | 773 |
| 10 | 1.6 | 730 | 9 | 2.2 | 773 |
| 10 | 1.8 | 730 | 10 | 1.2 | 773 |
| 10 | 2 | 730 | 10 | 1.3 | 773 |
| 10 | 2.25 | 730 | 10 | 1.4 | 773 |
| 10 | 2.5 | 730 | 10 | 2 | 773 |
| 11.5 | 1.6 | 730 | 10 | 2.4 | 773 |
| 12.3 | 1.625 | 730 | 11 | 1.8 | 773 |
| 12.5 | 1.26 | 730 | 9 | 1.8 | 866 |
| 12.5 | 1.6 | 730 | 11 | 1.5 | 866 |
| 6 | 2.7 | 731 | 6 | 1.85 | 1320 |
| 7 | 2.5 | 731 | 8 | 1.5 | 1320 |
| 8 | 2.3 | 731 | 10 | 1.25 | 1320 |
| 9 | 2.2 | 731 | 11 | 0.9 | 1320 |
| 9 | 2.5 | 731 | 12 | 0.7 | 1320 |
| 10 | 2 | 731 | 8 | 1.1 | 1500 |
| 10 | 2.25 | 731 | 8 | 1.5 | 1500 |
| 11 | 1.8 | 731 | 9.9 | 1.0 | 1500 |
| 11 | 2 | 731 | 9.9 | 1.5 | 1500 |
| 6 | 1.95 | 766 | 9.9 | 1.1 | 1500 |
| 8 | 1.65 | 766 | 10 | 1.0 | 1500 |
| 9 | 2.2 | 766 | 10 | 1.1 | 1500 |
| 10 | 1.3 | 766 | 10 | 1.25 | 1500 |
| 10 | 2 | 766 | 10 | 1.5 | 1500 |

TABLE 4

Compositions comprising poloxamer 338- demonstrated to form a gel at 37° C.

| wt% P338 | wt% HA | Mw HA kDa |
|---|---|---|
| 8 | 3 | 119 |
| 8 | 2 | 337 |
| 8 | 1.6 | 730 |
| 8 | 1.5 | 1500 |
| 10 | 2.5 | 119 |
| 10 | 1.75 | 337 |
| 10 | 1.4 | 730 |
| 10 | 1.1 | 1500 |

### Example 3. Rheological Properties of Exemplary Polymer Combination Preparations

The present Example 3 describes rheological properties of certain test polymer combination preparations as described in Example 1 and/or Example 2 above, as compared to those of reference chemically-crosslinked polymer biomaterials. Specifically, Example 3 describes storage modulus of certain test polymer combination preparations as described in Example 1 and/or Example 2 above, as compared to that of chemically-crosslinked hyaluronic acid biomaterials. As will be understood by a skilled artisan, methods for measuring storage modulus of biomaterials are known in the art. For example, in some embodiments, storage modulus of test and control biomaterials were measured using a rheometer with a parallel plate (e.g., a TA instruments Discovery HR2 rheometer using a 20 mm parallel plate, a 1,500 µm gap) at a frequency sweep of 0.1 Hz to 10 Hz, 0.4% strain at 37° C., soak time of 120 s and run time of 60 s. Results of storage modulus of certain test biomaterials are shown in Table 5 below:

TABLE 5

Certain Storage Modulus of Certain Biomaterial Combinations

| Polymer biomaterial | Storage modulus (G′, Pa) |
|---|---|
| 18% P407 | 15,750 |
| 13.5% P407 + 0.65% HA (10 mM PBS) | 8,200 |
| 13.5% P407 + 0.65% HA (0.1 M bicarbonate) | 7,800 |
| 13.5% P407 + 1.3% CMCH (10 mM PBS) | 900 |
| 10% P407 + 1% HA (10 mM PBS) | 200 |
| HyStem 12.5% Extralink | 1,000 |

In some embodiments, the storage modulus of hydrogels formed from exemplary polymer combination preparations (e.g. ones described herein) was not significantly different from the storage modulus of control 18% (w/w) P407 hydrogels at 37° C. In some embodiments, the storage modulus of hydrogels formed from exemplary polymer combination preparations (e.g. ones described herein) was about half of that of control 18% (w/w) P407 hydrogels. In some embodiments, the storage modulus of hydrogels formed from exemplary polymer combination preparations (e.g. ones described herein) was less than about 1/th of control 18% (w/w) P407 hydrogels at 37° C. In some embodiments, the storage modulus of hydrogels formed from exemplary polymer combination preparations (e.g. ones described herein) was about less than 1/100th that of control 18% (w/w) P407 hydrogels at 37° C. In specific embodiments, the storage modulus of hydrogels formed from exemplary polymer combination preparations (e.g. ones described herein) was about 8-10 kPa, about 7-9 kPa, about 6-8 kPa, about 5-7 kPa, about 4-6 kPa, about 3-5 kPa, about 2-4 kPa, about 1-3 kPa, about 500 Pa-2 kPa, about 1 kPa, or less than 1 kPa.

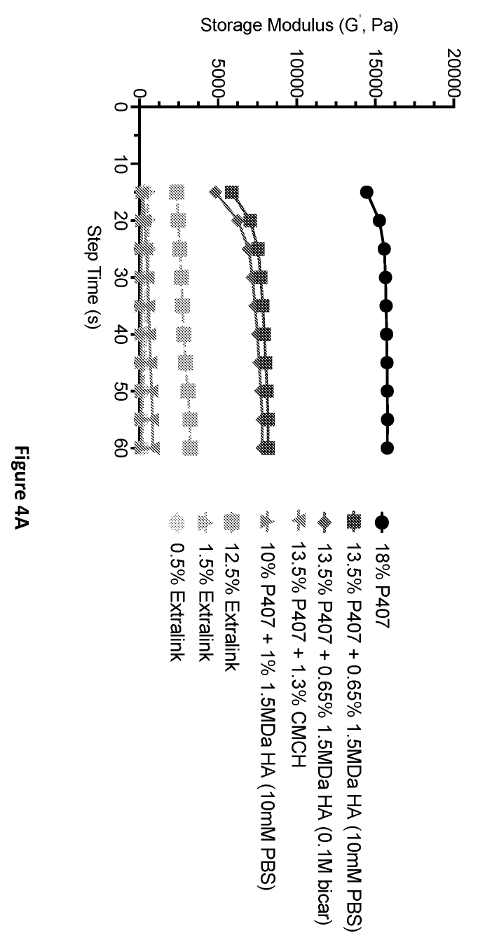

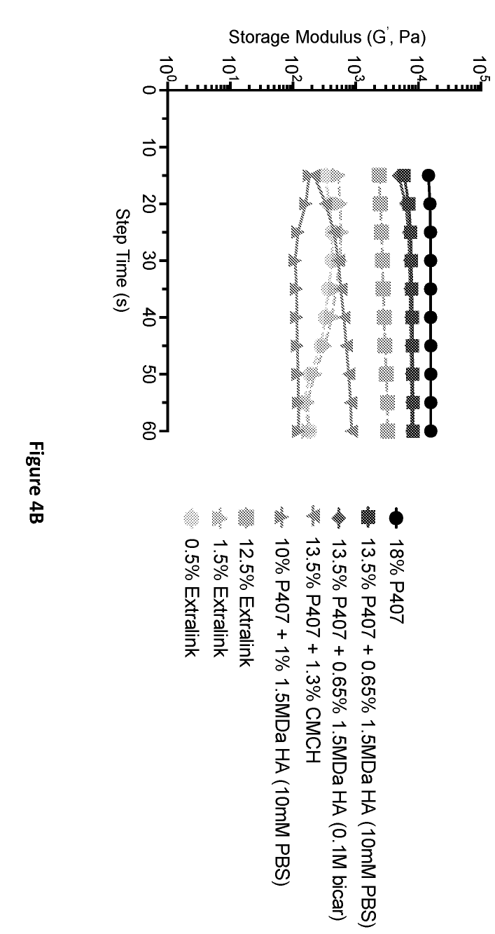

As shown in FIGS. 4A-4B, hydrogels formed from polymer combination preparations of P407 at a concentration of 13.5% or lower with hyaluronic acid or carboxymethyl chitosan have a lower storage modulus, e.g., by at least 30% or more, than that of a hydrogel formed from P407 at a concentration of 18% (w/w). FIGS. 4A-4B show hydrogels formed from polymer combination preparations of 10% P407 and 1% HA (1.5 MDa) or of 13.5% P407 and 1.3% carboxymethyl chitosan have a lower storage modulus, e.g., by at least 10% or more, than that of a chemically-crosslinked hyaluronic acid hydrogel with 12.5% Extralink thiol crosslinker (“HyStem”).

The storage stability of certain polymer combination preparations (e.g., ones described herein) were also assessed. For example, to assess the storage stability of polymer biomaterials, their storage moduli were measured over a period of time.

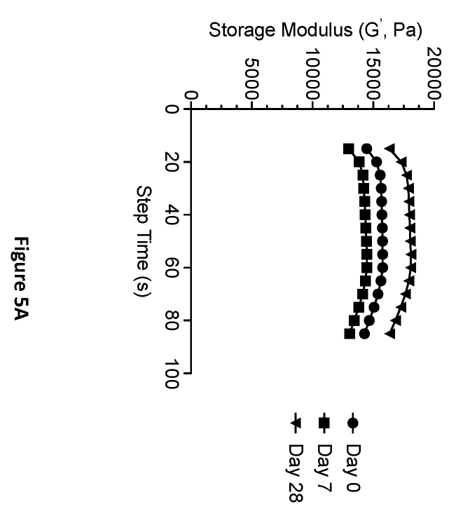

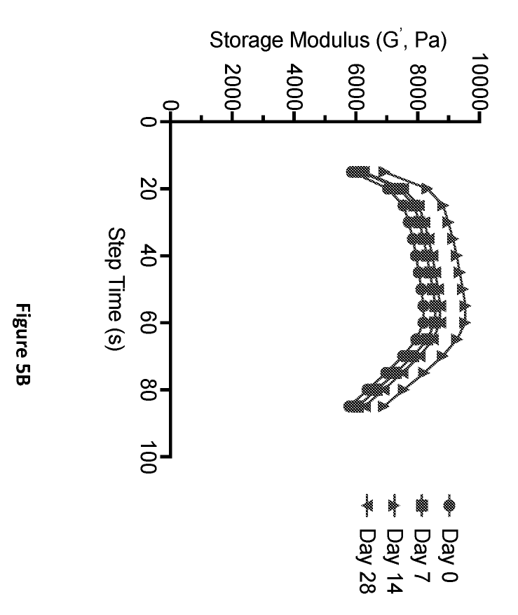

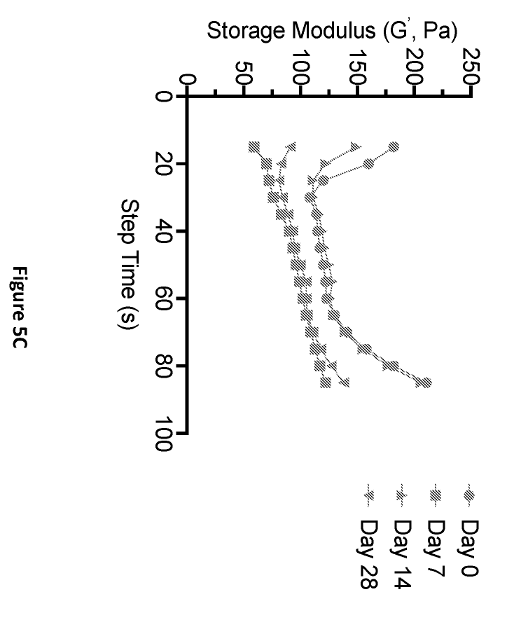

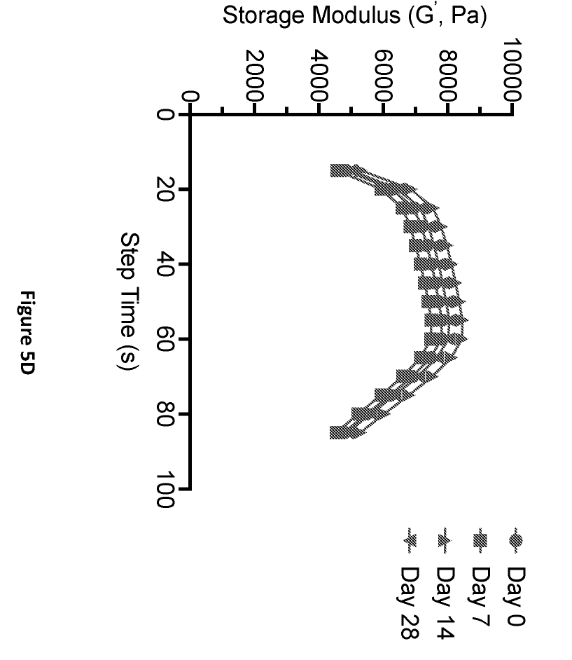

As shown in FIGS. 5A-5D, the storage stability of hydrogels formed from certain polymer combination preparations (e.g., ones described herein) are comparable to that of a reference biomaterial, e.g., a hydrogel formed from 18% (w/w) P407, as demonstrated by no significant change in their storage modulus over a period of about 1 month. In some embodiments, the storage modulus of hydrogels formed from exemplary polymer combination preparations (e.g. ones described herein) was largely stable over time. In some embodiments, the storage modulus of hydrogels formed from exemplary polymer combination preparations (e.g. ones described herein) was stable for one week, two weeks, three weeks, four weeks, or greater than four weeks.

### Example 4. Characterization of Exemplary Polymer Combination Preparations For Incorporation and Release of Active Agents

In some embodiments, exemplary polymer combination preparations can be useful to provide release of one or more payloads incorporated therein over a period of time. The present Example 4 describes characterization of certain test polymer combination preparations comprising Poloxamer 407 and a second polymer component, which may be or comprise a carbohydrate polymer (e.g., hyaluronic acid and/or chitosan or a variant thereof) with respect to release of a payload incorporated therein over a period of time. In some embodiments, an incorporated payload may be or comprise a hydrophilic agent. In some such embodiments, at least 20% (including, e.g., at least 30%, at least 40%, at least 50%, at least 60%, at least 70%, at least 80%, or more) of a hydrophilic agent incorporated agent may be released over a period of 6 hours, 12 hours, 18 hours, 24 hours, 48 hours, 72 hours, or longer. In some embodiments, an incorporated payload may be or comprise a lipophilic agent. In some such embodiments, at least 5% (including, e.g., at least 10%, at least 20%, at least 30%, at least 40%, at least 50% or more) of a lipophilic agent incorporated agent may be released over a period of 6 hours, 12 hours, 18 hours, 24 hours, 48 hours, 72 hours, or longer.

In some embodiments, hydrogels formed from exemplary polymer combination preparations (e.g. ones described herein) with an incorporated payload exhibit a payload release rate that is not significantly different from the payload release rate of control 18% (w/w) P407 hydrogels. In some embodiments, hydrogels formed from exemplary polymer combination preparations (e.g. ones described herein) with an incorporated payload exhibit a payload release rate that is slower than (e.g., by at least 10% or more) that of control 18% (w/w) P407 hydrogels. In some embodiments, hydrogels formed from exemplary polymer combination preparations (e.g. ones described herein) with an incorporated payload exhibit a payload release rate that is faster than (e.g., by at least 10% or more) than that of control 18% (w/w) P407 hydrogels. In some embodiments, hydrogels formed from exemplary polymer combination preparations (e.g. ones described herein) with an incorporated payload exhibit a payload release rate that is faster than (e.g., by at least 30% or more) than that of chemically-crosslinked hyaluronic acid hydrogels (with Extralink thiol crosslinkers).

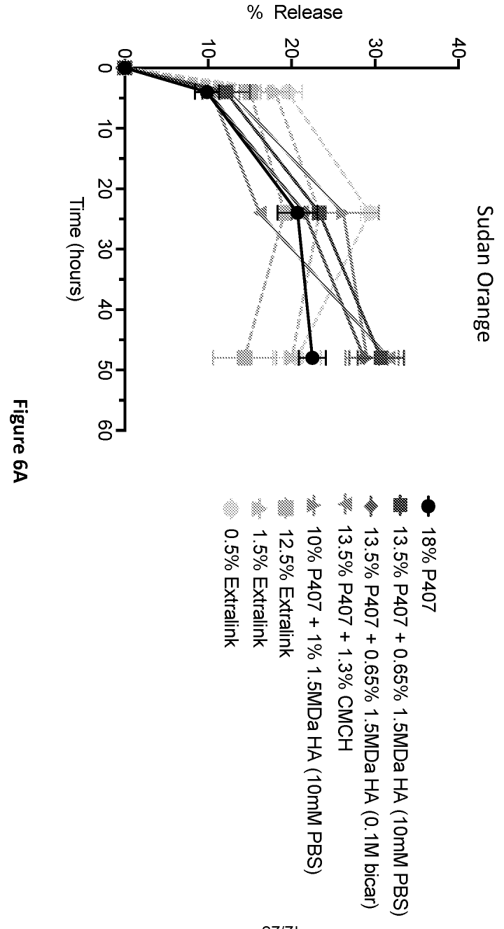

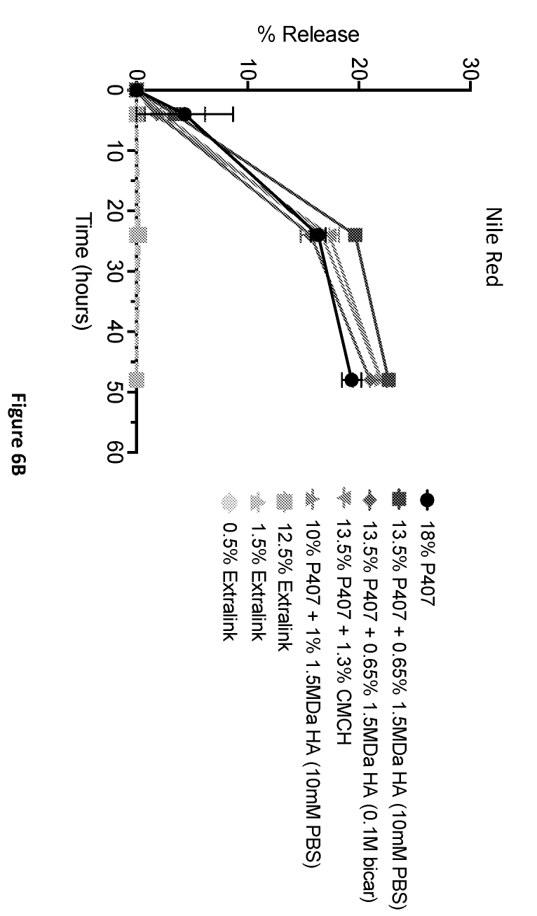

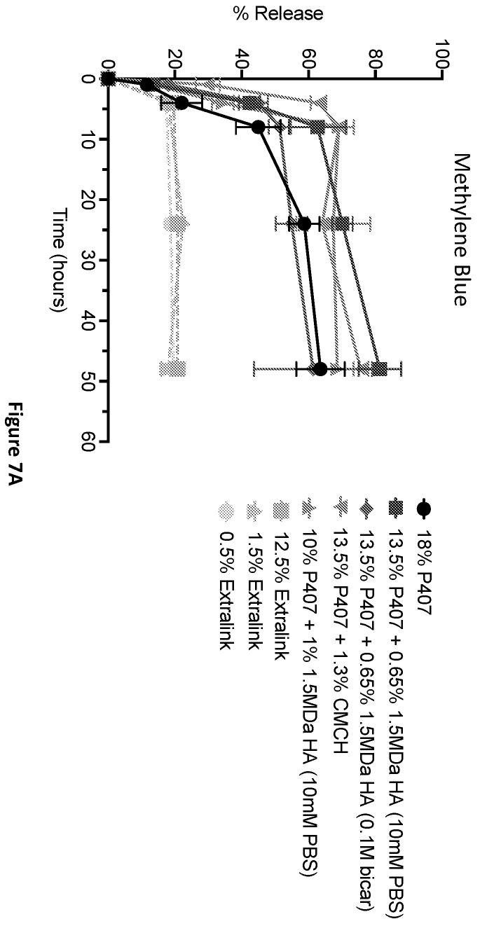

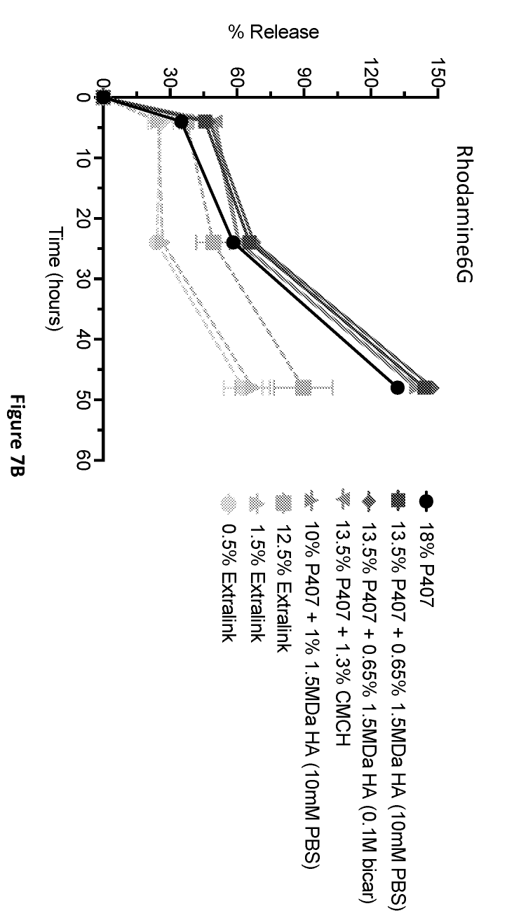

As described in Example 1, the release kinetics of an incorporated payload from a test polymer combination preparation hydrogel were assessed. FIGS. 6A-6B show in vitro release of exemplary lipophilic agents from exemplary temperature-responsive polymer combination preparations in a hydrogel state at a temperature of 37° C., while FIGS. 7A-7B show in vitro release of exemplary hydrophilic agents from exemplary temperature-responsive polymer combination preparations in a hydrogel state at a temperature of 37° C. These figures show that the chemically-crosslinked hyaluronic acid hydrogels (with Extralink thiol crosslinkers) typically released a hydrophilic agent more slowly that hydrogels formed from certain polymer combination preparations comprising poloxamer at a concentration lower than 18% and a carbohydrate polymer, even in some embodiments where such hydrogels formed from certain polymer combination preparations (e.g., comprising 10% w/w P407 and 1% w/w 1.5 MDa hyaluronic acid) may have a lower storage modulus than that of the chemically-crosslinked hyaluronic acid hydrogels. Without wishing to be bound by theory, release rate of such hydrogels (e.g., ones described herein) may be modulated by other properties other than storage modulus.

### Example 5. In Vivo Assessment of Exemplary Polymer Composition Preparations for Treatment of Tumor Resection Subjects

The present Example 5 demonstrates in vivo efficacy of certain polymer combination preparations comprising Poloxamer 407 and a second polymer component, which may be or comprise a carbohydrate polymer (e.g., hyaluronic acid and/or chitosan or a variant thereof) administered in tumor resection subjects (e.g., at a tumor resection site). In some embodiments, such polymer combination preparations may be administered alone in the absence of an immunomodulatory payload. In some embodiments, such polymer combination preparations may be incorporated with an immunomodulatory payload (e.g., a TLR7/8 agonist).

In some embodiments, a provided composition comprising a polymer combination preparation and an immunomodulatory payload is considered and/or determined to be useful for treatment of cancer (including, e.g., prevention or reduction in the likelihood of tumor relapse or metastasis) in accordance with the present disclosure when such a composition, after administration at a tumor resection site, reduces incidence of tumor recurrence and/or metastasis after the tumor resection (e.g., at least 1 month after tumor resection when the test subject is a mouse subject, or at least 3 months after tumor resection when the test subject is a human subject), for example, by at least 10% or more (comprising, e.g., at least 20%, at least 30%, at least 40%, at least 50%, at least 60%, at least 70%, at least 80%, at least 90%, or more), as compared to that which is observed when such a composition is not administered, or is administered without incorporation of an immunomodulatory payload.

In some embodiments, female BALB/cJs mice were inoculated orthotopically with 100,000 breast cancer cells (e.g., 4T1-Luc2 cells). Ten days later, tumors were surgically resected, and either (i) a composition described herein (e.g., comprising polymer combination preparation and an immunomodulatory payload such as, e.g., a TLR7/8 agonist (e.g., resiquimod), an NSAID (e.g., ketorolac), a pro-resolving mediator (e.g., Resolvin D2), an adenosine receptor antagonist (e.g., AB928), a Burton’s tyrosine kinase (BTK) inhibitor (e.g., Zanubrutinib), a CXCR4 signaling pathway inhibitor (e.g., Plerixafor), or (ii) a control composition (e.g., comprising polymer combination preparation without the immunomodulatory payload) was placed at a tumor resection site.

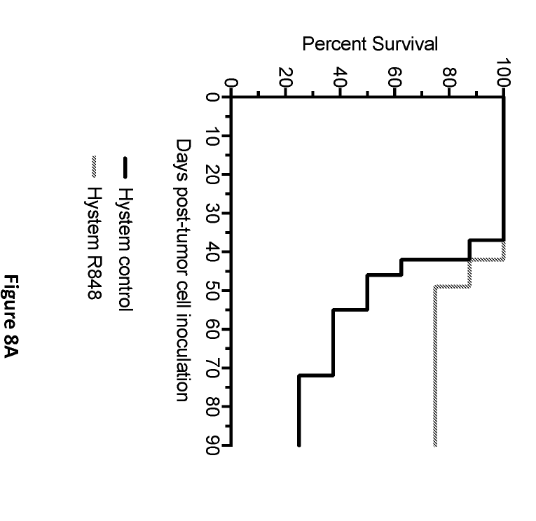

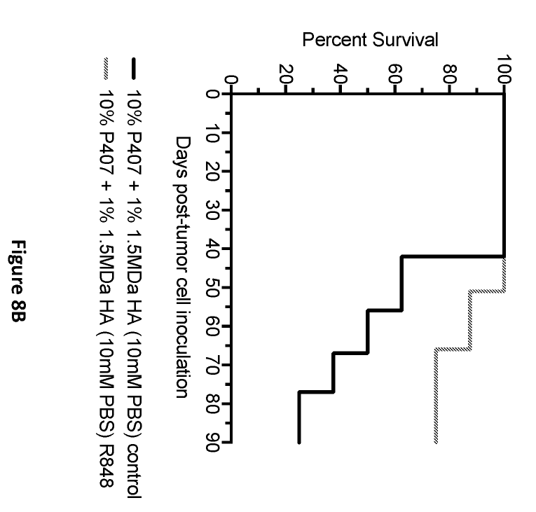

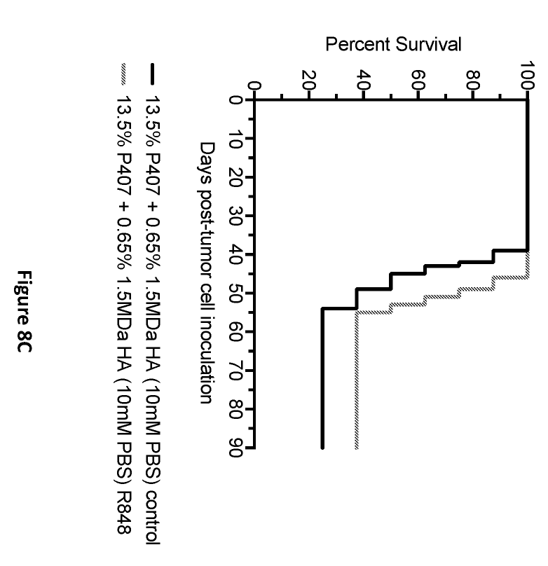

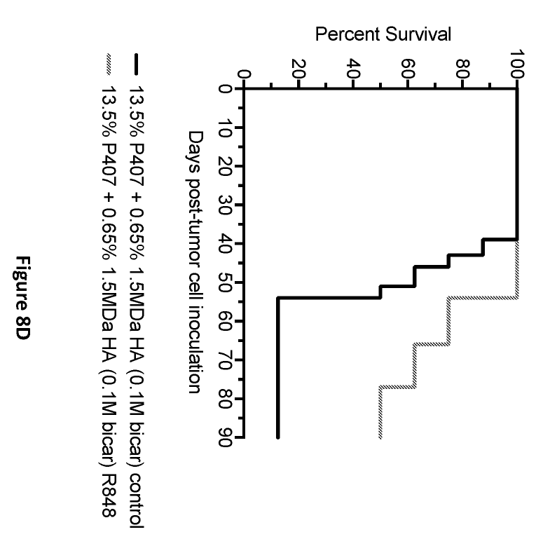

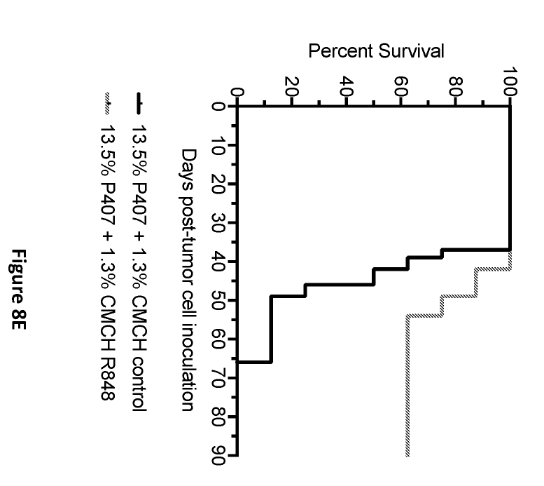

As shown in FIGS. 8A-8E, the group of tumor resection mice receiving a crosslinked hydrogel combination of 8-13.5% (w/w) or 6-11% (w/w) Poloxamer (e.g., 10% poloxamer e.g., poloxamer P407) and 0.6-1.5% (w/w) HA (e.g., 1% HA 1.5 MDa), incorporated with a TLR7/8 agonist (e.g., Resiquimod, R848), at a tumor resection site survived over a longer period of time, e.g., by at least 50% or more, as compared to the control group receiving the crosslinked hydrogel combination without the TLR7/8 agonist. In addition, the group of tumor resection mice receiving such a crosslinked hydrogel combination incorporated with TLR7/8 agonist (e.g., Resiquimod, R848) exhibited a much higher survival rate than the control group receiving the crosslinked hydrogel combination without the TLR7/8 agonist. Further, the efficacy of such a crosslinked hydrogel combination of 8-13.5% (w/w) Poloxamer (e.g., 10% poloxamer e.g., poloxamer P407) and 0.6-1.5% (w/w) HA (e.g., 1% HA 1.5 MDa), incorporated with a TLR7/8 agonist (e.g., Resiquimod, R848), was comparable to or better than that was observed with a chemically-crosslinked hyaluronic acid hydrogel (“HyStem”) incorporated with a TLR7/8 agonist (e.g., Resiquimod, R848).

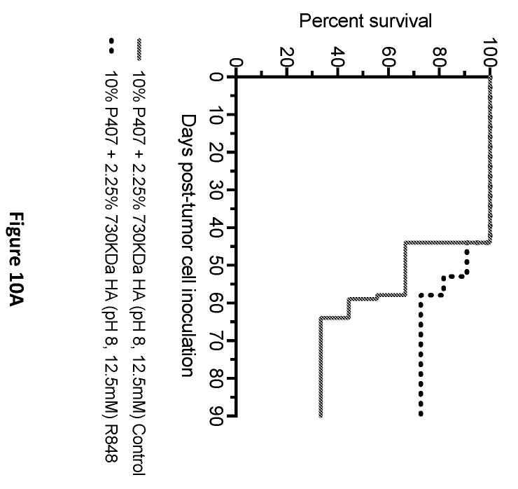

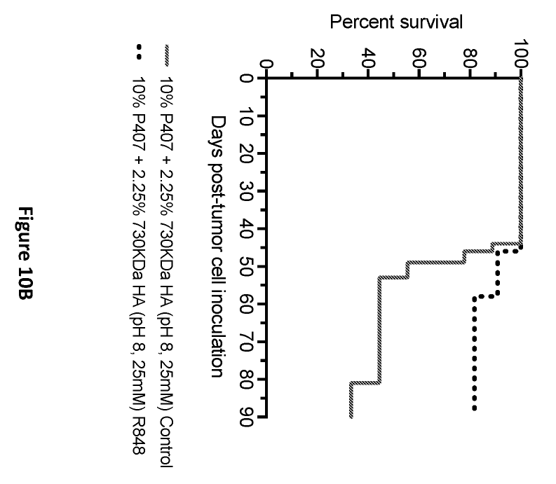

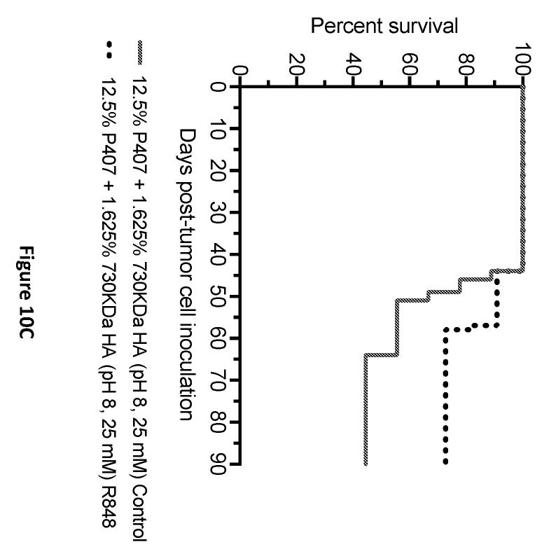

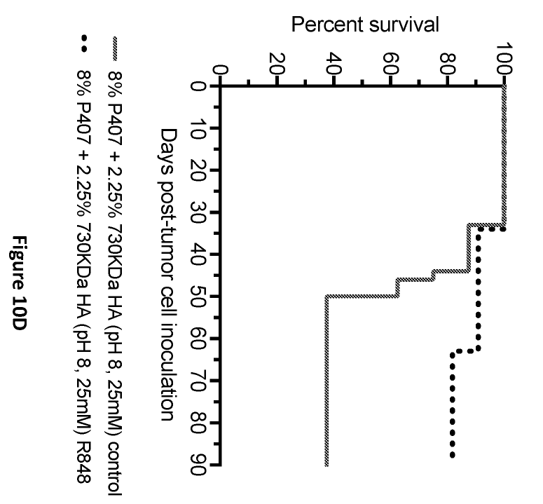

As shown in FIGS. 10A-10D the group of tumor resection mice receiving an immunomodulatory polymer combination of 8-12.5% (w/w) or 6-11% (w/w) poloxamer (e.g., 8%, 10% or 12.5% poloxamer, e.g., poloxamer P407) and 1.2-2.75% (w/w) HA (e.g., 1.625% or 2.25% HA 730 KDa), incorporated with a TLR7/8 agonist (e.g., Resiquimod, R848), at a tumor resection site survived over a longer period of time, as compared to the control group receiving an immunomodulatory polymer combination without the TLR7/8 agonist. In addition, the group of tumor resection mice receiving such an immunomodulatory polymer combination incorporated with TLR7/8 agonist (e.g., Resiquimod, R848) exhibited a higher survival rate than the control group receiving an immunomodulatory polymer combination without the TLR7/8 agonist.

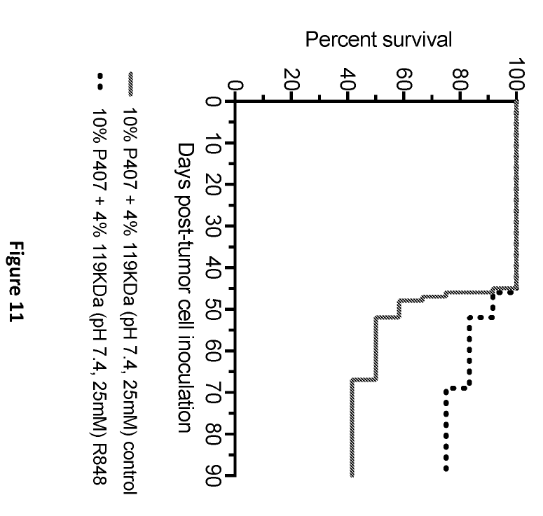

As shown in FIG. 11, the group of tumor resection mice receiving an immunomodulatory polymer combination of 8-12.5% (w/w) or 6-11% (w/w) poloxamer (e.g., 10% poloxamer e.g., P407) and 1-5% (w/w) HA (e.g., 4% HA 119 KDa), incorporated with a TLR7/8 agonist (e.g., Resiquimod, R848), at a tumor resection site survived over a longer period of time as compared to the control group receiving an immunomodulatory polymer combination without the TLR7/8 agonist. In addition, the group of tumor resection mice receiving such an immunomodulatory polymer combination incorporated with TLR7/8 agonist (e.g., Resiquimod, R848) exhibited a higher survival rate than the control group receiving an immunomodulatory polymer combination without the TLR7/8 agonist.

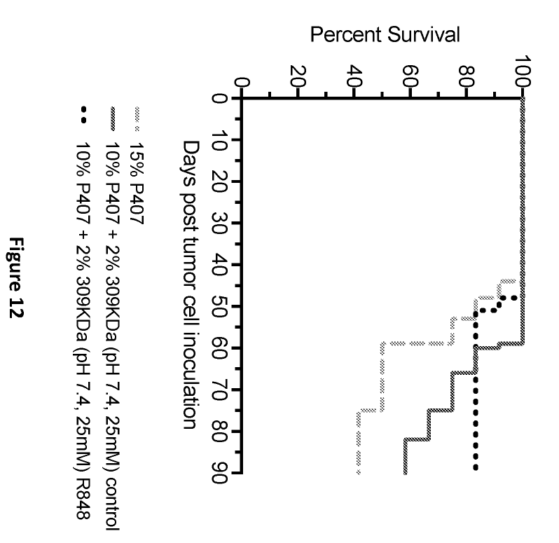

As shown in FIG. 12, the group of tumor resection mice receiving an immunomodulatory polymer combination of 8-12.5% (w/w) or 6-11% (w/w) poloxamer (e.g., 10% poloxamer e.g., P407) and 1-5% (w/w) HA (e.g., 2% HA 309 KDa), incorporated with or without a TLR7/8 agonist (e.g., Resiquimod, R848), at a tumor resection site survived over a longer period of time as compared to the control group receiving a 15% poloxamer only treatment without the TLR7/8 agonist.

These results demonstrated that in some embodiments, polymer combination preparations described herein are immunomodulatory and can be employed in the absence of an immunomodulatory payload, e.g., to treat subjects in need thereof, e.g., tumor resection subjects. The results also demonstrated that in some embodiments, polymer combination preparations described herein (e.g., immunomodulatory or not) can be used in combination with an immunomodulatory agent to treat subjects in need thereof, e.g., tumor resection subjects.

### Example 6. Identification and/or Characterization of Exemplary Immunomodulatory Polymer Combination Preparations

The present Example 6 describes identification and/or characterization of an immunomodulatory polymer combination preparation for antitumor efficacy, in particular by assessing its ability to extend survival of one or more subjects who have undergone a tumor resection. Accordingly, the present Example also describes identification and/or characterization of an immunomodulatory polymer combination preparation that may be useful for cancer treatment (e.g., as described herein). In some embodiments, such an immunomodulatory polymer combination preparation may induce innate immunity agonism. In some embodiments, such an immunomodulatory polymer combination preparation may resolve or reduce inflammation (e.g., immunosuppressive inflammation, such as in some embodiments immunosuppressive inflammation associated with wound healing).

In some embodiments, administration of an immunomodulatory polymer combination preparation to a target site following a tumor resection increases survival of a subject who has undergone a tumor resection, as compared to that observed when such an immunomodulatory polymer combination preparation is not administered.

In some embodiments, an animal model of cancer can be used to identify and/or characterize an immunomodulatory polymer combination preparation. For example, a tumor resection is performed on a tumor-bearing mouse, and a composition described herein, e.g., comprising an immunomodulatory polymer combination preparation in the absence of an immunomodulatory payload, is administered to the tumor resection site. Survival of treated subjects are then monitored. In some embodiments, an immunomodulatory polymer combination preparation is considered and/or determined to be useful in accordance with the present disclosure when it is characterized, in that when tested in vivo as described in the present Example, it extends survival of a treated subject, e.g., by at least 1 week, at least 2 weeks, at least 3 weeks, at least 4 weeks, at least 2 months, at least 3 months, at least 4 months, at least 5 months, at least 6 months, or longer, as compared to that observed in a control reference (e.g., a control in which an immunomodulatory polymer combination preparation is not administered). For example, in some embodiments, a control reference may be administration of a non-immunomodulatory polymeric biomaterial (e.g., a biomaterial of a poloxamer alone) in the absence of an immunomodulatory component. In some embodiments, a control reference may be administration of no polymeric biomaterial. In some embodiments, a control reference may be administration of a chemically-crosslinked biomaterial (e.g., a chemically-crosslinked HA, e.g., a thiolated HA).

In some embodiments, female BALB/cJ mice are inoculated orthotopically with 100,000 breast cancer cells (e.g., 4T1-Luc2 cells). Ten days later, tumors are surgically resected, and either (i) a composition described herein, e.g., comprising an immunomodulatory polymer combination preparation in the absence of an immunomodulatory payload (e.g., so that the immunomodulatory component of the composition consists essentially of or consists of the biomaterial), or (ii) a negative control composition (e.g., a buffered solution without such an immunomodulatory polymer combination preparation or a non-immunomodulatory polymeric biomaterial) is administered into the resection cavity. Animal survival can be monitored to inspect for induction of antitumor immunity. In some embodiments, to confirm that an administered immunomodulatory polymer combination preparation functions mechanistically, for example, by inducing innate immune signaling, animal survival may be monitored following neutralization of innate immune signaling (e.g., by administration of anti-IFNAR1). In some embodiments, to confirm that an administered immunomodulatory polymer combination preparation functions mechanistically by resolving or reducing inflammation (e.g. immunosuppressive inflammation associated with wound healing), animal survival may be monitored following neutralization of resolution of inflammation or anti-inflammatory effects.

To assess whether an administered immunomodulatory polymer combination preparation induces an adaptive antitumor immune response, animal survival may be monitored following depletion of particular leukocyte subsets (e.g., NK cells, CD4⁺ T cells, or CD8⁺ T cells).

In one aspect, the present Example 6 demonstrates administration of an immunomodulatory polymer combination preparation comprising (i) a chitosan or a variant thereof (e.g., carboxymethyl chitosan) as an innate immunity modulatory component and (ii) a temperature-responsive poloxamer (e.g., P407) that facilitates formation of a hydrogel when exposed to body temperature of a tumor resection subject (e.g., upon administration to a subject in need thereof) to a target site in a tumor resection subject improved survival of the tumor resection subject, as compared to that observed when such an immunomodulatory polymer combination preparation was not administered.

### Exemplary Liquid Preparations:

In some embodiments, a liquid preparation of an immunomodulatory polymer combination preparation (e.g., an innate immunity modulatory polymer combination preparation) was prepared as follows. For example, in one instance, a 2.5 weight percent (wt%) carboxymethyl chitosan (CMCH) (e.g., obtained from Heppe Medical Chitosan, Part Number 43002, Lot Number 312-210519-02) and Poloxamer 407 (P407) at a concentration of 12.5% or lower was prepared in a buffered system that is appropriate for injection administration. In another instance, a 5 wt% CMCH (e.g., obtained from Heppe Medical Chitosan, Part Number 43002, Lot Number 312-210519-02) and P407 at a concentration of 12.5% or lower was prepared in a buffered system that is appropriate for injection administration. For example, in some embodiments, such a buffered system has a physiological pH. The liquid preparation was loaded into a 1 mL syringe for administration. In some embodiments, 3-7 wt% low-molecular molecular weight (e.g., 100-120 kDa) hyaluronic acid and P407 at a concentration of 12.5% or lower was prepared in a buffered system that is appropriate for injection administration.

### Exemplary Mouse Tumor Models:

In some embodiments, animal experiments were performed using 6-8 weeks old female BALB/c mice (Jackson Laboratories, #000651). For animal survival studies, 10⁵ 4T1-Luc2 cells were inoculated orthotopically into the fourth mammary fat pad of a mouse. Tumor sizes were measured with calipers. Following size-matching, mice were randomly assigned to treatment groups, and surgery was performed on day 10 after tumor inoculation. For primary tumor resection, mice were anesthetized with 2% isoflurane, the tumor was resected, and an immunomodulatory polymer combination preparation (e.g., an innate immunity modulatory polymer combination preparation such as e.g., as described herein) that gels at body temperature was administered to a tumor resection site at the time of surgery.

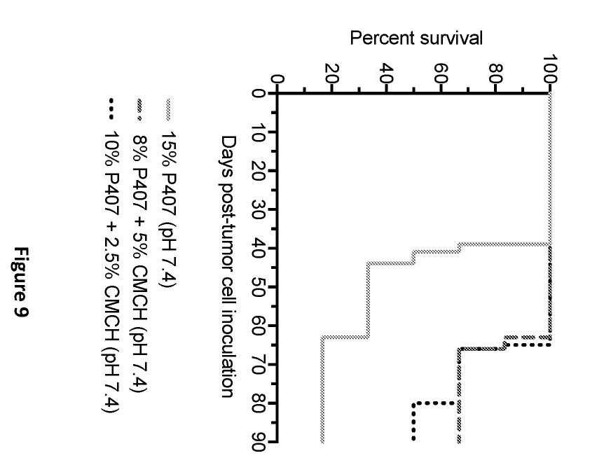

FIG. 9 shows survival data of tumor resection animals receiving a liquid preparation of an immunomodulatory polymer combination preparation (e.g., a liquid preparation of a combination of carboxymethyl chitosan at different concentrations and P407), as compared to tumor resection animals receiving a liquid preparation of a P407 alone. As demonstrated in FIG. 9, the group of tumor resection animals having in situ formation of a hydrogel combination of a poloxamer (e.g., P407) with carboxymethyl chitosan (CMCH) at a tumor resection site survived over a longer period of time, e.g., by at least 50% or more, as compared to the control group that did not receive such an immunomodulatory polymer combination preparation. In addition, the group of tumor resection animals receiving such a combination of a poloxamer (e.g., P407) with carboxymethyl chitosan (CMCH) exhibited a much higher survival rate than the control group without such an immunomodulatory polymer combination preparation. In some embodiments, such immunomodulatory polymer combination preparation may be useful for inducing innate immunity agonism.

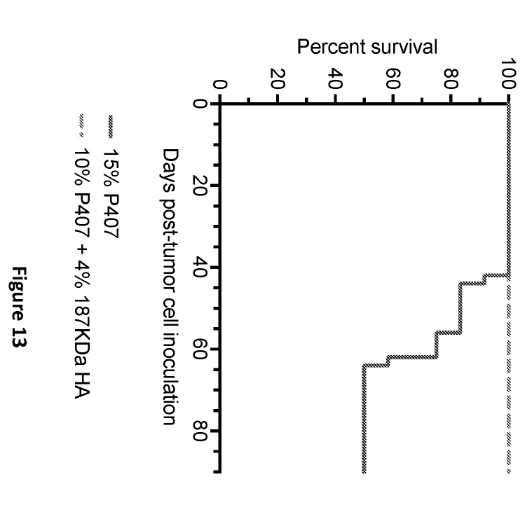

FIG. 13 shows survival data of tumor resection animals receiving a liquid preparation of an immunomodulatory polymer combination preparation (e.g., a liquid preparation of a combination of low molecular weight HA (e.g., 100-200 kDa) and P407), as compared to tumor resection animals receiving a liquid preparation of a P407 alone. As demonstrated in FIG. 13, the group of tumor resection animals having in situ formation of a hydrogel combination of a poloxamer (e.g., P407) with low molecular weight HA (e.g., 100-200 kDa) at a tumor resection site survived over a longer period of time as compared to the control group that did not receive such an immunomodulatory polymer combination preparation. In addition, the group of tumor resection animals receiving such a combination of a poloxamer (e.g., P407) with low molecular weight HA (e.g., 100-200 kDa) exhibited a much higher survival rate than the control group without such an immunomodulatory polymer combination preparation. In some embodiments, such immunomodulatory polymer combination preparation may be useful for inducing innate immunity agonism.

In another aspect, the present Example 6 describes that administration of an immunomodulatory polymer combination preparation (in the absence of a therapeutic payload) comprising (i) a high molecular weight HA (e.g., ones described herein such as in some embodiments HA weighing more than 500 kDa, e.g., ones described herein such as in some embodiments approximately 650 kDa HA, in some embodiments approximately 730 kDa HA, in some embodiments approximately 773 kDa, or in some embodiments approximately 1.5 MDa Ha) as an immunomodulatory component that can be useful for resolving or reducing inflammation (e.g., immunosuppressive inflammation) and (ii) a temperature-responsive poloxamer (e.g., P407) that facilitates formation of a hydrogel when exposed to body temperature of a tumor resection subject (e.g., upon administration to a subject in need thereof), to a target site in a tumor resection subject, can improve survival of the tumor resection subject, as compared to that observed when such an immunomodulatory polymer combination preparation is not administered. In some embodiments, such an immunomodulatory polymer combination can confer meaningful survival benefits among mice that have established spontaneous distal metastases. In some embodiments, such an immunomodulatory polymer combination preparation (in the absence of a therapeutic payload) may comprise 8%-12.5% P407 and 1%-5% HA 500 kDa. In some embodiments, such an immunomodulatory polymer combination preparation (in the absence of a therapeutic payload) may comprise 8%-12.5% P407 and 1%-3% HA 650 kDa. In some embodiments, such an immunomodulatory polymer combination preparation (in the absence of a therapeutic payload) may comprise 8%-12.5% P407 and 1%-3% HA 730 kDa. In some embodiments, such an immunomodulatory polymer combination preparation (in the absence of a therapeutic payload) may comprise 8%-12.5% P407 and 1%-3% HA 773 kDa. In some embodiments, such an immunomodulatory polymer combination preparation (in the absence of a therapeutic payload) may comprise 8%-12.5% P407 and 0.5%-2% HA 1.5 MDa.

In another aspect, the present Example 6 describes that administration of an immunomodulatory polymer combination preparation (in the absence of a therapeutic payload) comprising (i) a low molecular weight HA (e.g., ones described herein such as in some embodiments HA weighing less than 500 kDa, e.g., in some embodiments represented by HA comprising 100-400 kDa HA, e.g., 119 kDa HA, 187 kDa HA, 309 kDa HA, or 337 kDa HA) as an immunomodulatory component that can be useful for inducing or promoting inflammation (e.g., immunostimulatory inflammation) and (ii) a temperature-responsive poloxamer (e.g., P407) that facilitates formation of a hydrogel when exposed to body temperature of a tumor resection subject (e.g., upon administration to a subject in need thereof), to a target site in a tumor resection subject, can improve survival of the tumor resection subject, as compared to that observed when such an immunomodulatory polymer combination preparation is not administered.

In some embodiments, such an immunomodulatory polymer combination can confer meaningful survival benefits among mice that have established spontaneous distal metastases. In some embodiments, such an immunomodulatory polymer combination preparation (in the absence of an immunomodulatory payload) may comprise 8%-12.5% P407 or 6-10% P407 and 1%-5% HA 119 kDa or 5-9% HA 119 kDa. In some embodiments, such an immunomodulatory polymer combination preparation (in the absence of a therapeutic payload) may comprise 8%-12.5% P407 and 1%-5% HA 187 kDa. In some embodiments, such an immunomodulatory polymer combination preparation (in the absence of a therapeutic payload) may comprise 8%-12.5% P407 and 1%-5% HA 309 kDa. In some embodiments, such an immunomodulatory polymer combination preparation (in the absence of a therapeutic payload) may comprise 8%-12.5% P407 and 1%-5% HA 337 kDa.

### Example 7. Assessment of Gelation Properties of Additional Exemplary Polymer Combination Preparations

The present Example 7 describes assessment of gelation properties of certain test polymer combination preparations comprising Poloxamer 407 and a second polymer component, which may be or comprise a carbohydrate polymer (e.g., high molecular weight hyaluronic acid or a variant thereof).

The following Table 6 shows certain exemplary polymer combination preparations, and/or individual components thereof that are prepared in a suitable buffer (e.g., ones described herein) with pH around neutral pH.

TABLE 6

Certain exemplary polymer combination preparations

| wt% P407 | wt% HA | Mw HA kDa |
|---|---|---|
| 5 | 2 | 1320 |
| 5 | 2.1 | 1320 |
| 4 | 2.2 | 1320 |
| 4 | 2.3 | 1320 |
| 5 | 2.4 | 766 |
| 5 | 2.5 | 766 |
| 4 | 2.6 | 766 |
| 4 | 2.7 | 766 |

For example, in certain embodiments, polymer combination preparations, and/or individual components thereof can be prepared in a suitable buffer with pH 6-9. In some embodiments, polymer combination preparations, and/or individual components thereof can be prepared in a suitable buffer with pH 7-8. In some embodiments, polymer combination preparations, and/or individual components thereof can be prepared in a suitable buffer with pH 7.2-7.6. In some embodiments, polymer combination preparations, and/or individual components thereof can be prepared in a suitable buffer with pH 7.4. In some embodiments, polymer combination preparations, and/or individual components thereof can be prepared in a suitable buffer with pH 8.0.

In some embodiments, polymer combination preparations, and/or individual components thereof are prepared in a suitable buffer. In certain embodiments, polymer combination preparations, and/or individual components thereof are prepared in an aqueous buffer system. Examples of suitable aqueous buffer systems at an appropriate pH include, e.g., but are not limited to phosphate buffer at an appropriate pH. In some embodiments, polymer combination preparations, and/or individual components thereof are prepared in phosphate-buffered saline (PBS), sodium phosphate saline (SPS), potassium dihydrogen phosphate buffer, dipotassium hydrogen phosphate buffer, each at an appropriate pH. In some embodiments, polymer combination preparations, and/or individual components thereof are prepared in sodium phosphate 0.9% saline. In some embodiments, polymer combination preparations, and/or individual components thereof are prepared in an aqueous buffer system at a concentration within a range of 10 mM -50 mM or 20 mM-40 mM.

To assess gelation properties of various polymer combination preparations, each polymer preparation as described in Table 6 above is exposed to a target temperature for inducing gelation process (e.g., the body temperature of a subject such as a temperature of 37° C.) for a period of time (e.g., about 15-20 minutes) and then the physical state (e.g., solution vs. gel) of the polymer preparation is observed. Qualitative observations are made to determine gel formation characteristics. A polymer combination preparation is considered as forming a gel when the polymer combination preparation changes from a transparent solution to an opaque composition, when the preparation is observed to have no flow, and/or when a magnetic stir bar present in the polymer combination preparation does not move in the presence of a magnetic field.

For polymer preparations that form gels after exposure to a target gelation temperature, rheological analysis is performed, e.g., to determine storage modulus and/or phase angle of the resulting hydrogels.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

What is claimed is:

1. A preparation comprising:
a polymer combination preparation comprising at least first and second polymer components, the first polymer component is or comprises a poloxamer and the second polymer component is not a poloxamer, the polymer combination preparation being characterized in that it transitions from a precursor state to a polymer network state in response to a gelation trigger,
wherein the polymer network state has a viscosity materially above that of the precursor state,
wherein the gelation trigger is or comprises temperature at or above critical gelation temperature (CGT) for the polymer combination preparation, ratio of polymer components at or above critical gelation weight ratio for the at least first and second polymer components, molecular weights of the at least first and/or second polymer components, or combinations thereof;
wherein the polymer network state comprises crosslinks not present in the precursor state;
wherein the crosslinks are or comprise intra-molecular crosslinks, inter-molecular crosslinks, or both; and
wherein the first polymer component is present in the polymer combination preparation is present at a concentration of 12.5% (w/w) or below.

2. The preparation of claim 1, wherein the crosslinks do not comprise covalent crosslinks.

3. The preparation of claim 2, wherein the CGT for the polymer combination preparation is 30-39° C. or 20-30° C.

4. The preparation of any one of claims 1-3, wherein the polymer combination preparation comprises a total polymer content of at least 5% (w/w), or at least 10% (w/w).

5. The preparation of any one of claims 1-4, wherein the critical gelation weight ratio of the first polymer component to the second polymer component is 1:1 to 14:1 or 1:1 to 10:1.

6. The preparation of any one of claims 1-5, wherein the polymer network state is a viscous solution or colloid.

7. The preparation of any one of claims 1-5, wherein the polymer network state is a hydrogel.

8. The preparation of claim 7, wherein the polymer network state is characterized in that, when tested in vitro at 37° C., the polymer combination preparation releases a lipophilic agent incorporated therein at a comparable rate as with a hydrogel formed from a P407 solution with a concentration of 18% (w/w).

9. The preparation of any one of claims 1-8, wherein the polymer network state is characterized in that, when tested in vitro at 37° C., more than 60% of a lipophilic agent incorporated in the polymer combination preparation can be retained therein for at least 24 hours.

10. The preparation of any one of claims 1-8, wherein the polymer network state is characterized in that, when tested in vitro at 37° C., no more than 40% of a lipophilic agent incorporated in the polymer combination preparation can be released therefrom within 24 hours.

11. The preparation of any one of claims 1-10, wherein the polymer network state is characterized in that, when tested in vitro at 37° C., the polymer combination preparation releases a hydrophilic agent incorporated therein at a comparable rate as, or at a faster rate than, that of a hydrogel formed from a P407 solution with a concentration of 18% (w/w).

12. The preparation of any one of claims 1-11, wherein the polymer network state is characterized in that, when tested in vitro at 37° C., the polymer combination preparation releases a hydrophilic agent incorporated therein at a faster rate (e.g., by at least 20% within 48 hours) as compared with a chemically crosslinked hyaluronic acid hydrogel.

13. The preparation of claim 12, wherein the chemically crosslinked hyaluronic acid hydrogel is a hydrogel formed by mixing thiol-modified hyaluronic acid (Glycosil®) with a crosslinking agent, thiol-reactive PEGDA crosslinker (Extralink®) under conditions for gelation to occur.

14. The preparation of any one of claims 1-13, wherein the polymer network state is characterized in that when tested in vitro at 37° C., at least 40% of a hydrophilic agent incorporated in the polymer combination preparation is released therefrom within 12 hours.

15. The preparation of any one of claims 1-14, wherein the second polymer component is or comprises a carbohydrate polymer.

16. The preparation of claim 15, wherein the carbohydrate polymer in the polymer combination preparation is present at concentration of below about 10% (w/w) or below about 5% (w/w).

17. The preparation of claim 15 or 16, wherein the carbohydrate polymer is or comprises hyaluronic acid.

18. The preparation of claim 17, wherein the hyaluronic acid has a molecular weight of about 50 kDa to about 2 MDa.

19. The preparation of claim 18, wherein the hyaluronic acid has a low molecular weight of about 100-500 kDa or about 100-400 kDa, or about 125-375 kDa, or about 100-200 kDa.

20. The preparation of claim 18, wherein the hyaluronic acid has a high molecular weight of about 500-1,500 kDa or about 600-800 kDa.

21. The preparation of claim 18, wherein the carbohydrate polymer is or comprises chitosan or a modified chitosan.

22. The preparation of claim 21, wherein the modified chitosan is or comprises carboxymethyl chitosan.

23. The preparation of any one of claims 1-22, wherein the polymer network state is characterized by one or more material properties selected from:
a. storage modulus within a range of 100 Pa to about 10,000 Pa as measured at 37° C. and pH 5-8;
b. storage modulus that is at least 40% lower than that of a hydrogel formed from a solution having a poloxamer at a solution concentration of 18% (w/w); and
c. storage modulus, as measured at 37° C., that maintains substantially the same after its precursor state has been stored at 2-8° C. for a period of 1 month (or no more than 20% of the polymer combination preparation is degraded over a period of 1 month, when measured at 37° C.).

24. The preparation of any one of claims 1-23, wherein the polymer combination preparation has pH 4.5-8.5.

25. The preparation of any one of claims 1-24, wherein the polymer combination preparation has pH 7-8 (e.g., pH 7.4).

26. The preparation of any one of claims 1-25, wherein the polymer combination preparation has a higher buffering capacity than a 10 mM phosphate buffer.

27. The preparation of any one of claims 1-26, wherein the poloxamer is or comprises Poloxamer 407.

28. The preparation of any one of claims 1-27, further comprising a therapeutic agent.

29. The preparation of claim 28, wherein the therapeutic agent is or comprises an analgesic, antibiotic, anticoagulant, antiemetic, coagulant, or agent that promotes wound healing.

30. The preparation of any one of claims 1-28, wherein the therapeutic agent is or comprises an immunomodulatory payload.

31. The preparation of claim 30, wherein the immunomodulatory payload is or comprises a modulator of innate immunity.

32. The preparation of claim 30 or 31, wherein the immunomodulatory payload is or comprises a modulator of myeloid cell function.

33. The preparation of any one of claims 30-32, wherein the immunomodulatory payload is or comprises a modulator of adaptive immunity.

34. The preparation of any one of claims 30-32, wherein the immunomodulatory payload is or comprises a modulator of inflammation.

35. The preparation of claim 34, wherein the immunomodulatory payload is or comprises a TLR7/8 agonist.

36. The preparation of claim 34, wherein the immunomodulatory payload is or comprises resiquimod.

37. The preparation of claim 34, wherein the immunomodulatory payload is or comprises a COX2 inhibitor.

38. The preparation of claim 34, wherein the immunomodulatory payload is or comprises an NSAID, e.g., ketorolac.

39. The preparation of any one of claims 1-38, wherein when the second polymer component is or comprises a carbohydrate polymer and the polymer combination preparation is substantially free of an immunomodulatory payload, the polymer combination preparation is characterized in that a test animal group with spontaneous metastases having, at a tumor resection site, the polymer combination preparation in the polymer network state has a higher percent survival than a comparable test animal group having, at a tumor resection site, a poloxamer biomaterial in the absence of the second polymer component, as assessed at 2 months after the administration.

40. The preparation of any one of claims 1-39, wherein when the polymer combination preparation comprises an immunomodulatory payload, the polymer combination preparation is characterized in that a test animal group with spontaneous metastases having, at a tumor resection site, the polymer combination preparation in the polymer network state has a higher percent survival than a comparable test animal group having, at a tumor resection site, a polymer combination preparation without the immunomodulatory payload, as assessed at 2 months after the administration.

41. The preparation of claim 39 or 40, wherein the polymer combination preparation in the polymer network state is delivered to the tumor resection site by intraoperatively administering at the tumor resection site a preparation comprising the polymer combination preparation that is pre-formed in the polymer network state.

42. The preparation of claim 39 or 40, wherein the polymer combination preparation in the polymer network state is delivered to the tumor resection site by intraoperatively administering at the tumor resection site a preparation comprising the polymer combination preparation that is in the precursor state, which transitions to the polymer network state at the tumor resection site after the administration.

43. The preparation of any one of claims 1-42, wherein the first polymer component is present in the polymer combination preparation at a concentration of 11% (w/w) or below.

44. The preparation of any one of claims 1-42, wherein the first polymer component is present in the polymer combination preparation at a concentration of 10.5% (w/w) or below.

45. The preparation of any one of claims 1-42, wherein the first polymer component is present in the polymer combination preparation at a concentration of 10% (w/w) or below.

46. The preparation of any one of claims 1-45, wherein the first polymer component is present in the polymer combination preparation at a concentration of at least 4% (w/w), at least 5% (w/w), or at least 6% (w/w).

47. A method comprising administering the preparation of any one of claims 1-46 to a subject in need thereof.

48. The method of claim 47, wherein the subject in need thereof is a subject suffering from cancer.

49. The method of claim 48, wherein the subject in need thereof is a subject suffering from or susceptible to recurrent or disseminated cancer.

50. The method of any one of claims 47-49, wherein the subject in need thereof is a tumor resection subject.

51. The method of claim 50, wherein the preparation is administered at or within 2 cm of the tumor resection site.

52. The method of any one of claims 49-51, wherein the administration is by implantation.

53. The method of claim 52, wherein a preparation comprising the polymer combination preparation in the polymer network state is administered by implantation.

54. The method of any one of claims 49-51, wherein the administration is by injection.

55. The method of claim 54, wherein a preparation comprising the polymer combination preparation in the precursor state is administered by injection, wherein the precursor state transitions to the polymer network state upon the administration.

56. The method of any one of claims 49-55, wherein the administration is performed concurrently with or subsequent to laparoscopy.

57. The method of any one of claims 49-55, wherein the administration is performed concurrently with or subsequent to minimally invasive surgery.

58. The method of any one of claims 49-55, wherein the administration is performed concurrently with or subsequent to robotic surgery.
